(19) United States
(12) Patent Application Publication
(10) Pub. No.: US 2023/0194505 A1
(43) Pub. Date: Jun. 22, 2023

(54) DETERMINING DRUG EFFECTS USING COMBINATION OF MEASUREMENTS

(71) Applicant: Organos, Inc., Berkeley, CA (US)

(72) Inventors: Aslak Tveito, Berkeley, CA (US); Karoline Horgmo Jaeger, Berkeley, CA (US); Samuel Wall, Berkeley, CA (US)

(73) Assignee: Organos, Inc., Berkeley, CA (US)

(21) Appl. No.: 17/925,943

(22) PCT Filed: May 17, 2021
(86) PCT No.: PCT/US2021/032799

§ 371 Date: Nov. 17, 2022

Related U.S. Application Data
(60) Provisional application No. 63/026,400, filed on May 18, 2020.

(51) Int. Cl. G01N 33/50

(52) U.S. Cl. CPC G01N 33/5061; G01N 33/5035

(57) ABSTRACT

Measurements in test tissue and drug-treated tissue can be used to determine an effect of the drug, e.g., on cardiomyocytes. Optical measurements of test tissue (e.g., including immature or animal CMs) can be complemented with observations of the extracellular potential, e.g., using multi electrode arrays (MEAs), as part of accurately estimating the current density of an ion channel, e.g., the sodium channels. The estimating of an ion current density (e.g., by inversion) of the ion (e.g., sodium) current can also use an observation of the conduction velocity, which can be computed using measurements of extracellular waves across electrodes. Example optical measurements can be of a transmembrane voltage (Vm) and intracellular calcium concentration (Cai). An example electrical measurement can be of an extracellular potential (U).

### CROSS-REFERENCES TO OTHER APPLICATIONS

This application claims priority to U.S. Provisional Patent Application No. 63/026,400, entitled “Determining Drug Effects Using Combination Of Measurements” filed on May 18, 2020, which is hereby incorporated by reference in its entirety for all purposes.

### BACKGROUND

The discovery of human induced pluripotent stem cells (hiPSCs) has started a new era in biological science and medicine. These reprogrammed somatic cells can be differentiated into a wide variety of cell lineages, and allow in vitro examination of cellular properties at the level of the human individual. In particular, this technology has large implications in drug development, moving us away from well-studied but often unrepresentative animal models towards direct testing of compounds in specific human phenotypes and genotypes. This new access offers the potential for creating better, safer drug treatments; both from the ability to target precision, patient-specific approaches, and to reveal possible side effects of drugs in the broader human population. However, despite its promise, the technology needed to fully utilize hiPSCs for drug testing is still under development and currently faces many difficulties limiting practical applicability.

In particular, the problem of maturation is a major challenge to the successful use of hiPSCs. Although hiPSCs can be used to create specialized human cells and tissues, these rapidly grown cells and tissues may have significant proteomic and structural differences to, and are often more fetal-like than, their adult in vivo counterpart. This is especially true in hiPSC derived cardiomyocytes (hiPSC-CMs), where the adult cells they are intended to represent have undergone decades of growth and development under cyclical physiological loading and stimulation.

Cardiomyocytes derived from human induced pluripotent stem cells (hiPSC-CMs) offer a new means to study and understand the human cardiac action potential, and can give key insight into how compounds may interact with important molecular pathways to destabilize the electrical function of the heart. Important features of the action potential can be readily measured using standard experimental techniques, such as the use of voltage sensitive dyes and fluorescent genetic reporters to estimate transmembrane potentials and cytosolic calcium concentrations. Using previously introduced computational procedures, such measurements can be used to estimate the current density of major ion channels present in hiPSC-CMs, and how compounds may alter their behavior.

However, due to the limitations of optical recordings, resolving the sodium current remains difficult from these data.

### BRIEF SUMMARY

Embodiments of the present disclosure can provide methods, systems, and apparatuses for determining an effect of a drug on a type of cells. Measurements in test tissue and drug-treated tissue can be used to determine an effect of the drug, e.g., on cardiomyocytes. In some examples, optical measurements of test tissue (e.g., including immature or animal CMs) can be complemented with observations of the extracellular potential, e.g., using multi electrode arrays (MEAs), as part of accurately estimating the current density of an ion channel, e.g., the sodium channels. The estimating of an ion current density (e.g., by inversion) of the ion (e.g., sodium) current can also use an observation of the conduction velocity, which can be computed using measurements of extracellular waves across electrodes.

Example optical measurements can be of a transmembrane voltage (Vₘ) and intracellular calcium concentration (Caᵢ). An example electrical measurement can be of an extracellular potential (U). The combined data including the membrane voltage/potential (Vₘ), the cytosolic calcium concentration (Caᵢ), and the extracellular potential (U) further allows for the possibility of accurately estimating the effect of novel drugs applied to heart cells (e.g., hiPSC-CMs, animal CM cells, or mature human CM cells).

These and other embodiments of the disclosure are described in detail below. For example, other embodiments are directed to systems, devices, and computer readable media associated with methods described herein.

A better understanding of the nature and advantages of embodiments of the present disclosure may be gained with reference to the following detailed description and the accompanying drawings.

### BRIEF DESCRIPTION OF THE DRAWINGS

FIG. 1 shows an example geometry used in the bidomain-base model simulations according to embodiments of the present disclosure.

FIGS. 2 and 3A-3C show a simulation procedure used to generate data for the inversions according to embodiments of the present disclosure.

FIG. 4 illustrates some of the properties included in the cost function (8) according to embodiments of the present disclosure.

FIG. 5 shows an effect of perturbing I_{Kr}, I_{CaL} and I_{Na} on the V, Ca and U data according to embodiments of the present disclosure.

FIG. 6 shows an effect of perturbing I_{Kr}, I_{CaL} and I_{Na} on the computed conduction velocity according to embodiments of the present disclosure.

FIG. 7 shows the effect of block of the I_{Kr}, I_{CaL} and I_{Na} currents for three different extracellular calcium concentrations according to embodiments of the present disclosure.

FIG. 8 illustrates how including an extra extracellular calcium concentration adjustment may improve the identifiability of I_{Kr} and I_{CaL} according to embodiments of the present disclosure.

FIGS. 9 and 10 show a repolarization wave in hiPSC-CM data and bidomain-base model simulations according to embodiments of the present disclosure.

FIGS. 11 and 12 shows measured and simulated U data in 64 electrodes for the control case and for 10 μM Flecainide according to embodiments of the present disclosure.

FIG. 13 shows a result of the inversion procedure applied to simulated data of twelve drugs, based on the block percentages corresponding to three times the free plasma Cₘₐₓ reported in [7] according to embodiments of the present disclosure.

FIG. 14 shows a result of the inversion procedure applied to simulated data of the twelve drugs of FIG. 13 according to embodiments of the present disclosure.

FIG. 15 is a flowchart of a method according to embodiments of the present disclosure.

FIG. 16 shows dog (left) and human (right) ventricular action potentials in the control case and in the presence of 50 nM of the I_{Kr} blocker dofetilide. The dotted lines show measured data from [N Jost et al., The Journal of Physiology, 591(17):4189-4206, 2013], and solid lines show simulation results. Note that the drug effect used in the simulation of the human AP was estimated from the dog data.

FIG. 17 shows zerbrafish (left) and human (right) ventricular action potentials in the control case and in the presence of 50 nM of the I_{Kr} blocker dofetilide.

FIG. 18 shows Upper left; illustration of an animal cardiomyocyte that express three types of ion-channels (proteins) in the myocyte membrane surface (sarcolemma). Two of the proteins are also present in the membrane of the human cardiomyocyte (upper right). Note that the human ventricular myocyte also express an additional type of protein not present in the animal cell. In the lower panels, both the animal and the human cardiomyocytes are subjected to a blocking drug that binds to one of the protein types. An essential assumption for allowing quantitative translation of animal measurements to estimates of the effect of human cells is that, at the level of a single protein, the effect of the drug is independent of whether the protein is expressed in human or an animal cell membrane.

FIG. 19 is a flowchart of a method according to embodiments of the present disclosure.

FIG. 20 shows a block diagram of an example computer system usable with systems and methods according to embodiments of the present invention.

### TERMS

As used herein, the following terms have the meanings ascribed to them unless specified otherwise.

The terms “a,” “an,” or “the” as used herein not only include aspects with one member, but also include aspects with more than one member. For instance, the singular forms “a,” “an,” and “the” include plural referents unless the context clearly dictates otherwise. Thus, for example, reference to “a cell” includes a plurality of such cells and reference to “the agent” includes reference to one or more agents known to those skilled in the art, and so forth.

The terms “about” and “approximately” as used herein shall generally mean an acceptable degree of error for the quantity measured given the nature or precision of the measurements. Typical, exemplary degrees of error are within 20 percent (%), preferably within 10%, and more preferably within 5% of a given value or range of values. Any reference to “about X” specifically indicates at least the values X, 0.95X, 0.96X, 0.97X, 0.98X, 0.99X, 1.01X, 1.02X, 1.03X, 1.04X, and 1.05X. Thus, “about X” is intended to teach and provide written description support for a claim limitation of, e.g., “0.98X.”

As used herein, the term “drug” refers to any molecule or composition that produces a pharmacological effect on a cell, tissue, organ, body system, or whole organism, e.g., when a sufficient amount is contacted with a cell, tissue, or organ, or when a sufficient amount is administered to a subject. The term covers molecules or compositions that are known or expected to exert an effect on cardiac cells or tissues, as well as molecules or compositions that are not known or expected to exert an effect on cardiac cells or tissues. In some embodiments, a drug exerts an effect on one or more proteins in a cardiac cell or tissue (e.g., transmembrane and/or sarcoplasmic reticulum proteins such as ion channels, exchangers, pumps, release proteins, and/or uptake proteins that permit the passage of ions (e.g., sodium, potassium, calcium, magnesium, chloride, bicarbonate, protons, hydroxide ions, or other relevant ions), proteins, or other species). In some embodiments, a drug is a sodium channel blocker, a calcium channel blocker, a calcium channel blocker, or a combination thereof. Drugs may be synthetic or naturally occurring. The term includes prodrugs as well.

As used herein, the term “mature cardiomyocyte” or “mature CM” refers to an adult cardiomyocyte. In contrast, the term “immature cardiomyocyte” or “immature CMf” refers to a non-adult CM or a CM that is not fully differentiated, e.g., a fetal cardiomyocyte or a cardiomyocyte derived from a stem cell such as a pluripotent stem cell. In some instances, the immature CM is derived from a human pluripotent stem cell (hPSC-CM). In some embodiments, the immature cardiomyocyte is an induced pluripotent stem cell-derived cardiomyocyte (iPSC-CM). In some embodiments, the immature CM is derived from a cell (e.g., a stem cell such as a pluripotent stem cell) that is obtained from a subject (e.g., a subject that is to be administered, or potentially administered a drug that is assessed for proarrhythmic effects according to methods of the present invention).

Mature and immature CMs can be differentiated from each other in a number of ways, including but not limited to differences in morphology, electrophysiology, and the expression of various proteins. For example, mature CMs tend to be rod-shaped, whereas immature CMs tend to be rounded. In addition, mitochondria in mature CMs tend to be oval-shaped, whereas mitochondria in immature CMs and smaller than those found in mature CMs and/or are more rounded in shape. Mature CMs typically exhibit synchronous contraction, whereas immature CMs typically do not. Mature CMs tend to exhibit faster upstroke velocities (e.g., around 250 mV/ms) and faster conduction velocities (e.g., about 60 cm/s) than immature CMs (e.g., upstroke velocities of about 50 mV/ms and conduction velocities of about 10-20 cm/s (for hPSC-CMs)). Immature CMs also tend to have a more depolarized resting membrane potential (e.g., about −60 mV) as compared to mature CMs (e.g., about −90 mV).

In mature CMs, various ion transporters, sarcomeric proteins, and calcium handling proteins are upregulated as compared to immature CMs. Non-limiting examples include KCNA4, KCNA5, KCNAB1, KCNAB2, KCND2, KCND3, KCNE4, KCNG1, KCNH2, KCNH7, KCNIP2, KCNJ2, KCNJ3, KCNJ5, KCNJ8, KCNK1, KCNQ1, KCNV1, SCN1A, SCN1B, SCN2B, SCN3A, SCN4B, SCN5A, HCN1, HCN4, CACNAlC, CACNA1D, CACNAlH, CACNA1G, CACNA2D1, CACNB2, SLC8A1, TRPC3, TRPC4, TRPC6, CFTR, ATP2A2, PLN, CASQ2, RYR2, RYR3, TRDN, ITPR1, ITPR3, ASPH, S100A1, HRC, MYL2, TNNI3, ACTN2, MYH7, MYL3, TNNC1, TNNT2, MYH11, SORBS1, CASQ2, RyR, SERCA, and L-type calcium channels. In addition, certain proteins are downregulated in mature CMs, including uMHC, T-type calcium channels, Titin, and N2BA. Additional information regarding differences between mature and immature CMs can be found, e.g., in Jiang et al. Mol. Cells (2018) 41(7):613-621, hereby incorporated by reference in its entirety for all purposes.

Unless specified otherwise, the term “transmembrane voltage” or “Vₘ” refers to the difference in electrical potential (e.g., in millivolts (mV)) between the interior and exterior of a cell (e.g., a cardiomyocyte), and is determined with respect to the exterior of the cell (i.e., a negative Vₘ means that the interior of the cell is negative with respect to the exterior of the cell).

Unless specified otherwise, the term “intracellular calcium” or “Caᵢ” refers to cytosolic calcium ions, and does not include calcium ions located in the endoplasmic reticulum, sarcoplasmic reticulum, or mitochondria. In particular embodiments, the term also excludes calcium that is not in ionized for and/or calcium that is bound to a protein or chelator.

Unless specified otherwise, the term “extracellular potential” refers to the measured electrical potential between an electrode in contact with the extracellular space above or under the cardiomyocytes and a reference electrode in contact with the cell media but not in contact with cells.

The term “proarrhythmic” refers to any effect that results in an increased likelihood that an arrhythmia will occur. Evidence of proarrhythmic effects may be observed at the cellular level (e.g., decreased (dv/dt)max, prolongation of the APD_{V,n} or APD_{Ca,n}, increased Caᵢ, depolarization of the resting membrane potential, etc.), tissue level (e.g., decreased conduction velocity, increased automaticity, etc.), or organ level (e.g., prolongation of the QT or corrected QT interval, repolarization abnormalities that manifest as changes in T wave morphology on electrocardiogram, etc.). The arrhythmia may be an atrial arrhythmia, a ventricular arrhythmia, a reentrant arrhythmia, or a combination thereof. In some embodiments, a proarrhythmic effect is caused by modification of the function or properties of a cardiac cell protein (e.g., ion channel, pump, exchanger, SR release protein, or SR uptake protein) by a drug. As a non-limiting example, proarrhythmic effects can be caused by decreased ion channel conductance that is secondary to interactions between a drug and the ion channel.

The term “APD_{V,n}” refers to the duration of the Vₘ action potential (e.g., measured in milliseconds) and is taken to be the time from when the value of the transmembrane potential, in the upstroke, is n % below its maximum value (to) until it is again repolarized to n % of its maximum value (t). The term “APD_{Ca,n}” represents the corresponding transient duration (e.g., in milliseconds) for the intracellular calcium concentration. The value of n can be any number between 0 and 100, excluding 0 (e.g., 5, 10, 15, 20, 25, 30, 35, 40, 45, 50, 55, 60, 65, 70, 75, 80, 85, 90, or 95). In some embodiments, n is 30, 50, or 80.

The terms “subject,” “individual,” and “patient” are used interchangeably herein to refer to a vertebrate, preferably a mammal, more preferably a human. Mammals include, but are not limited to, rodents (e.g., mice, rats), simians, humans, farm animals, sport animals, and pets.

### DETAILED DESCRIPTION

Embodiments can determine an effect of a drug on a type of cells. Measurements in test tissue and drug-treated tissue can be used to determine an effect of the drug, e.g., on cardiomyocytes. The measurements on the test tissue can be used to determine parameter values pr c for a control tissue model, e.g., by updating parameters p^{T,B} in a base tissue model. The measurements on the drug-treated tissue can be used to update the parameter values p^{T,C} to obtain the parameter values p^{T,D} for a drug-treated tissue model. A change between one or more parameter values p^{T,D} and one or more parameter values p^{T,C} can provide an effect of the drug.

In some examples, optical measurements of test tissue (e.g., including immature or animal CMs) can be complemented with observations of the extracellular potential, e.g., using multi electrode arrays (MEAs), as part of accurately estimating the current density (an example of a parameter) of an ion channel, e.g., the sodium channels. The estimating of an ion current density (e.g., by inversion) of the ion (e.g., sodium) current can also use an observation of the conduction velocity, which can be computed using measurements of extracellular waves across electrodes.

Example optical measurements can be of a transmembrane voltage (Vₘ) and intracellular calcium concentration (Caᵢ). An example electrical measurement can be of an extracellular potential (U). The combined data including the membrane voltage/potential (Vₘ), the cytosolic calcium concentration (Caᵢ), and the extracellular potential (U) further allows for the possibility of accurately estimating the effect of novel drugs applied to heart cells (e.g., hiPSC-CMs, animal CM cells, or mature human CM cells).

As examples, the test cells can be immature or animal CMs. Regarding animal tissue, using animal cells and tissues as precise measuring devices for developing new drugs presents a long-standing challenge for the pharmaceutical industry. Despite the very significant resources that continue to be dedicated to animal testing of new compounds, only qualitative results can be obtained. This often results in both false positives and false negatives. Here, we show how the effect of drugs applied to animal ventricular myocytes can be translated, quantitatively, to estimate a number of different effects of the same drug on human cardiomyocytes. We illustrate and validate our methodology by translating, from animal to human, the effect of dofetilide applied to dog cardiomyocytes, the effect of E-4031 applied to zebrafish cardiomyocytes, and, finally, the effect of sotalol applied to rabbit cardiomyocytes. In all cases, the accuracy of our quantitative estimates are demonstrated. Our computations reveal that, in principle, electrophysiological data from testing using animal ventricular myocytes, can give precise, quantitative estimates of the effect of new compounds on human cardiomyocytes.

### I. INTRODUCTION

In recent reports [1, 2], we have demonstrated how microphysiological systems utilizing human induced pluripotent stem derived cardiomyocytes (hiPSC-CMs)[3, 4] can be used to estimate drug induced changes to the cardiac action potential using computational approaches. These methods use optical measurements of the membrane potential and the cytosolic calcium concentration to quantitate changes in underlying ion channel conductances and calcium handling pathways using a mathematical model of the of hiPSC-CMs dynamics. We have further shown how these estimates, at least in principle, carry over from immature cells to adult cardiomyocytes. This methodology provides information on a number of the major ion channels. When compared to data presented in [5, 6, 7, 8, 9, 10, 11], the method is able to provide reasonable estimates of the IC50 values of well-known drugs like Nifedipine, Lidocaine, Cisapride, Flecainide and Verapamil; see Table 3 of [2]. These drugs affect the I_{CaL}, I_{NaL} or I_{Kr} currents, and the effects are well estimated by our methodology, e.g., as described in U.S. Patent Publication No. 2019/0242879, which is incorporate by reference in its entirety for all purposes.

However, difficulties remain in the characterization of the fast sodium current, I_{Na}. This is a major issue since this current more or less completely governs the rapid upstroke of the action potential and thus also the conduction velocity. Therefore, it is of great importance to characterize the effect of drugs on this current. The reason for this deficiency in our methodology is the time resolution of the data obtained by fluorescence; the data used in the inversions are provided with a resolution of 10 ms and this is far too coarse to be able to estimate the strength of I_{Na}. Time resolution can be improved but at the cost of less accurate data and therefore another experimental technique is needed to pin down the channel density of and drug effects on I_{Na}. The extracellular potential U can be measured in microphysical systems using multielectrode arrays; see e.g., [12, 13, 14, 15, 16].

In this disclosure, we show that the extracellular data can be used to determine the sodium current. And therefore, by combining imaging data (or other measurements) for the membrane potential (V) and the cytosolic calcium concentration (Ca) with data (e.g., electrical measurements) for the extracellular potential (U), we are able to identify both the fast sodium current and other major currents characterizing the action potential of the hiPSC-CMs.

A main challenge in combining V, Ca, and U data is that a spatial problem needs to be resolved. When the data are given by V and Ca only, we have used a data trace obtained by taking the average over the whole chip (see [3, 1]), and the inversion of the data has amounted to estimating parameters describing a system of ordinary differential equations. But when U is added to the data, the extracellular potential needs to be included in the model. In our present implementation, we use the bidomain model (see e.g. [17]) for this purpose. The bidomain model has been used for inversion of U data (but not V and Ca) by several authors; see [18, 16, 19, 20, 14].

However, it is demonstrated in [18] that the bidomain model does not provide an extracellular repolarization wave (a spatial difference in the time at which cells repolarize). Such a wave is clearly present in the experimental data, and inhomogeneities would have to be introduced in the bidomain model in order to enforce a repolarization wave. These inhomogeneities are difficult to obtain from measurements, and therefore some embodiments use U only to estimate the currents involved in generating the upstroke and not the whole action potential. Such a technique can determine I_{Na} accurately in data generated by simulations.

### II. COMBINATION OF MEASUREMENTS (OPTICAL AND ELECTRICAL)

In this section, we describe methods that can be applied to identify drug response by combining measurements of the membrane potential V, the cytosolic calcium concentration Ca, and the extracellular potential U in microphysiological systems of hiPSC-CMs. Techniques use a tissue model involving multiple cells as opposed to a single cell model. Embodiments can use a bidomain model as the tissue model.

Simulations using a tissue model can be performed as a substitute for physical measurements to confirm viability of the technique. Parameters (e.g., sodium current) are assumed and used to generate simulated measurements for the membrane potential, the cytosolic calcium concentration, and the extracellular potential. Then, those measurements are used in an inversion procedure to confirm that the output parameter values (i.e., determined via an inversion procedure) are similar to the input parameter values. The inversion procedure can solve a differential equation to determine V, Ca, and U, and the parameters can be varied to obtain a best match in the predicted values for V, Ca, and U relative to the simulated or measured values for V, Ca, and U. This formulation of the tissue model can use a continuum approach, e.g., where the underlying differential equations can be solved using various basis functions, such as fine difference or finite elements, to determine predicted values for V, Ca, and U for a given set of input parameters.

A. Bidomain-Base Model Simulations

In order to represent the electrical properties of a microphysiological system, we conduct simulations of the bidomain model of the form

$$χ ( {C}_{m} \frac{\partial v}{\partial t} + {I}_{i o n} ( v , s ) ) = ∇ \cdot ( {σ}_{i} ∇ v ) + ∇ \cdot ( {σ}_{i} ∇ {u}_{e} ) , \qquad ( 1 )$$

$$0 = ∇ \cdot ( {σ}_{i} ∇ v ) + ∇ \cdot ( ( {σ}_{i} + {σ}_{e} ) ∇ {u}_{e} ) , \qquad ( 2 )$$

$$\frac{\partial s}{\partial t} = F ( v , s ) \qquad ( 3 )$$

(see e.g., [21, 17, 22, 23]). Here, v and uₑ are the membrane potential and the extracellular potential, respectively. In addition, σᵢ and σₑ are the bidomain conductivities of the extracellular and intracellular spaces, respectively, which describe the degree to which the respective spaces conduct current. Cₘ is the specific membrane capacitance, and χ is the surface-to-volume ratio of the cell membrane. To determine simulated measurements, the values chosen for these parameters are given in Table 1.

TABLE 1
Parameter values used in the bidomain-base model simulations.

| Parameter | Value | |
|---|---|---|
| Cₘ | 1 | μF/cm ² |
| σᵢ | 0.4 | mS/cm |
| σₑ | 10 | mS/cm |
| X | 1400 | cm⁻¹ |
| Domain size | 1600 μm × 1600 μm | |
| Electrode size | 50 μm × 50 μm | |
| Electrode distance | 100 | μm |
| Δx, Δy | 50 | μm |
| Δt (recordings of V and Ca) | 10 | ms |
| Δt (extracellular recordings) | 0.05 | ms |

| | |
|---|---|
| *Δt (simulations) | 0.05 ms for t ≤ 50 ms |
| | 0.2 ms for t > 50 ms |

Furthermore, Iᵢₒₙ represents the density of currents through different types of ion channels, pumps and exchangers on the cell membrane. We use an adjusted version of the hiPSC-CM base model introduced in [2] to represent these currents. The current density is then given on the form

Iᵢₒₙ(v,s)=ΣᵢIᵢ(v,s), (4)

where each Iᵢ represents the current through a specific type of ion channel, pump or exchanger. In an example model, these channels can be: I_{Na}, the fast sodium channel, I_{NaL}, the late sodium channel, Iₜₒ, the transient outward sodium channel, I_{Kr} the rapidly, and I_{Ks}, the slowly activating delayed rectifying potassium currents, I_{K1}, the inward rectifying potassium current, I_{f}, the funny current, I_{bCl} and I_{bCa} the background chloride and calcium currents respectively, and I_{CaL}, the L-type calcium current.

The hiPSC-CM base model includes a number of additional state variables representing the gating of ion channels and intracellular Ca²⁺ concentrations. These variables are represented by s in the model above, and their dynamics are modeled by a set of ordinary differential equations (ODEs) given by F(v, s). A number of parameters in the hiPSC-CM base model have been adjusted to make the size of eight currents of particular interest (I_{Kr}, I_{NaL}, I_{CaL}, I_{Na}, Iₜₒ, I_{Ks}, I_{K1}, and I_{f}) close to the size of the currents in the model described in [24], which is fitted to recordings of hiPSC-CMs from several different studies. The adjusted parameter values of the hiPSC-CM base model are given in Table 2.

Table 2 shows parameter values used in the hiPSC-CM base model. The remaining parameter
values are the same as specified in [2]. These parameters govern the magnitude of the currents

| Parameter | Value | | Parameter | Value | |
|---|---|---|---|---|---|
| [Ca²⁺]ₑ | 0.42 | mM | Ī_{NaK} | 1.9 | μA/μF |
| [K+]ₑ | 5 | mM | Ī_{NaCa} | 9.4 | μA/μF |
| [Na⁺]ₑ | 140 | mM | Ī_{pCa} | 0.12 | μA/μF |
| g_{Na} | 2.6 | mS/μF | J̅_{SERCA} | 0.00016 | mM/ms |
| g_{NaL} | 0.03 | mS/μF | α_{RyR} | 0.0052 | ms⁻¹ |
| gₜₒ | 0.21 | mS/μF | β_{RyR} | 0.0265 | mM |
| g_{Kr} | 0.075 | ms/μF | α_{d}^{c} | 0.0027 | ms⁻¹ |
| g_{Ks} | 0.0127 | mS/μF | αₛₗ^{c} | 0.3 | ms⁻¹ |
| g_{K1} | 0.05 | mS/μF | αₙ^{s} | 0.0093 | ms⁻¹ |
| g_{f} | 0.012 | mS/μF | Bₜₒₜ^{c} | 0.063 | mM |
| g_{bCl} | 0.0001 | mS/μF | Bₜₒₜ^{d} | 2.7 | mM |
| g_{bCa} | 0.0005 | mS/μF | Bₜₒₜ^{sl} | 1.45 | mM |

| | | | | |
|---|---|---|---|---|
| g_{CaL} | 1.8 nL/(μF/ms) | Bₜₒₜ^{s} | 60 | mM |

The

$$\frac{\partial v}{\partial t}$$

on the left-hand side of equation (1) is a cell model that conveys what is changing across the cell membrane. Changes in the left-hand side is equal to the opposite change that happens outside of the cell there needs to be a balance. The changes outside of the cell are defined with respect to transmembrane potential V (also labeled v), the extracellular potential U (also labeled uₑ), and the bidomain conductivities of the extracellular and intracellular spaces. For example, as current flows into the cell during the heartbeat, there has to be a proportional change in the space outside the cell.

The calcium concentration can be determined via equation (3), which is a set of ordinary differential equations that describe the workings of the cell. F is a function of the transmembrane voltage and is an ordinary interpreter equation, so

$$\frac{\partial s}{\partial t}$$

is a function of s. With s being the current state of the system, equation (3) progresses that state over time so the change of the state is a function of the current state. The solution of these three equations provides the transmembrane V, the extracellular potential U, and the calcium concentration Ca. Since it is a tissue model, one can determine how quickly the current goes through the system, and thus the conduction velocity can be determined. The conduction velocity is a fourth measurement that can be used in the inversion procedure to obtain more accurate parameter(s) of a model. The conduction velocity can be measured in the system and is an emergent property of the model that depends on the parameterization (i.e., can be determined from the parameters, which may be optimized based on measurements).

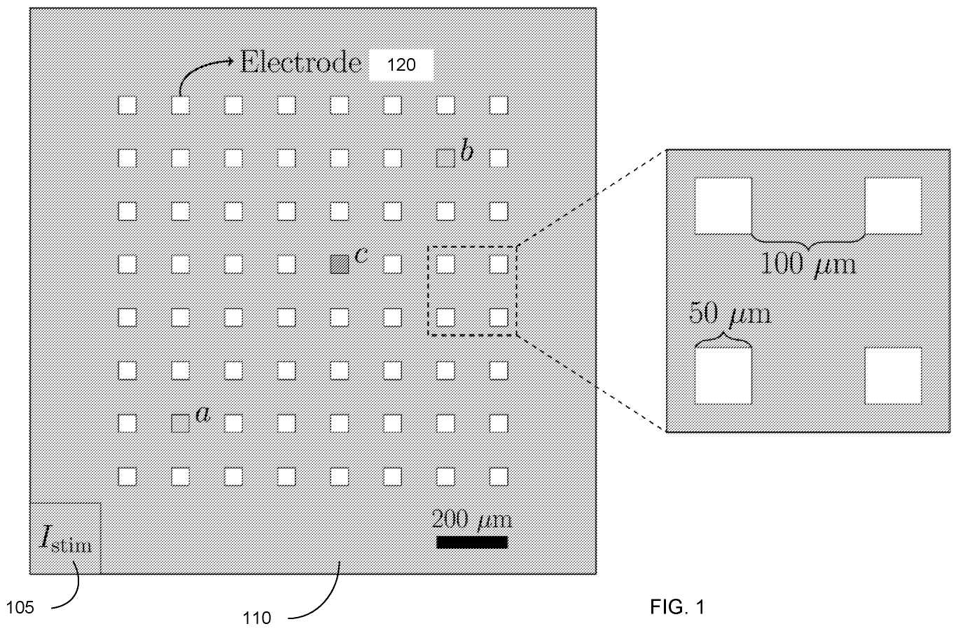

The geometry of the domain used to represent a microphysiological system is described in FIG. 1 and Table 1. We consider a two-dimensional domain, and initiate a travelling wave by stimulating the lower left corner, shown as Iₛₜᵢₘ 105. Furthermore, 8×8 electrodes are distributed in the center of the domain, and we record the extracellular potential in these electrodes. On the boundary of the domain, we apply the Dirichlet boundary condition uₑ=0.

In addition to the bidomain simulations, in some cases, we conducted simulations in which the spatial variation of the variables are ignored. In that case, the system (1)-(3) can be written as a pure ODE system of form

$$\frac{dv}{dt} = - \frac{1}{{C}_{m}} {I}_{ion} , \qquad ( 5 )$$

$$\frac{ds}{dt} = F ( v , s ) . \qquad ( 6 )$$

Below, we refer to the system (5)-(6) as the pure ODE system. The extracellular potential is omitted in the pure ODE system.

1. Adjustment Factors

In order to investigate whether we are able to identify drug responses from combined measurements of V, Ca and U data, we perform numerical simulations representing a number of different drugs reported in [7]. More specifically, we simulate ion channel blockers by introducing adjustment factors λᵢ≤0, such that Iᵢₒₙ is given by

Iᵢₒₙ(v,s)=Σᵢ(1+λᵢ)Iᵢ(v,s). (7)

The adjustment factors λ are also referred to as blocking factors.

FIG. 1 shows an example geometry used in the bidomain-base model simulations according to embodiments of the present disclosure. The domain 110 contains 8×8 evenly spaced electrodes 120, and the propagating wave is initiated by stimulating the lower left corner of the domain, shown as Iₛₜᵢₘ 105. The conduction velocity is computed between the electrodes marked as a and b. When we plot the extracellular potential of just a single electrode, we consider the electrode marked as c. Domain 110 corresponds to an area of the cells. The dimensions of the electrodes and distances between them are examples dimensions. Different dimensions may be used.

In our simulation, we consider the drug effect on three major ionic currents, I_{Kr}, I_{CaL} and I_{Na}, believed to be of importance in evaluation of drug safety (see e.g., [7]), and we therefore introduce the three adjustment factors A_{Kr}, A_{CaL} and λ_{Na}.

2. Bidomain-Base Model Simulations Used to Generate Data

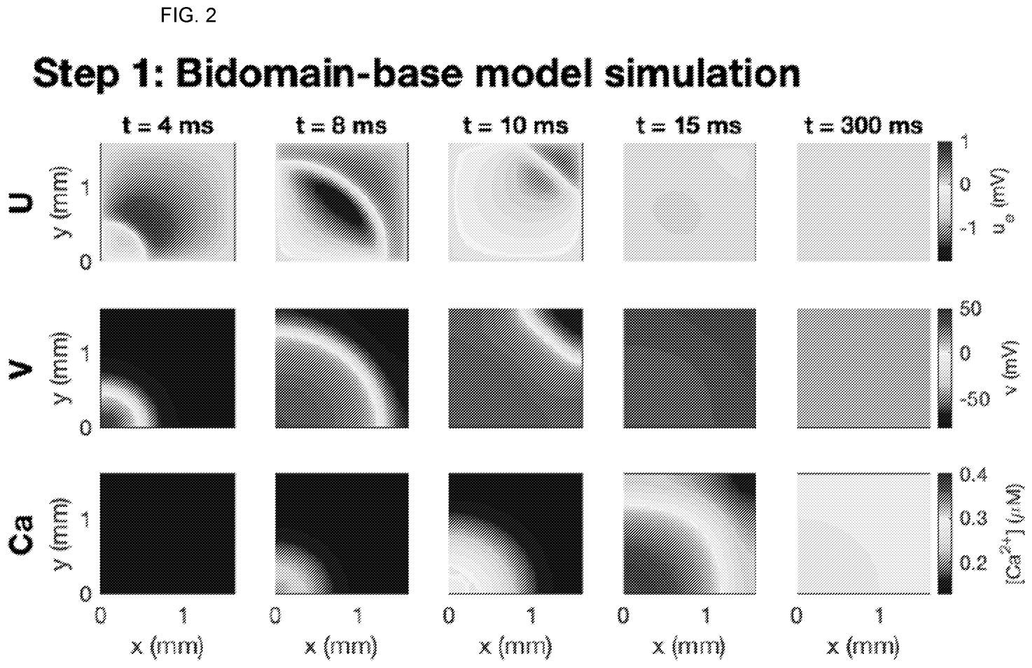

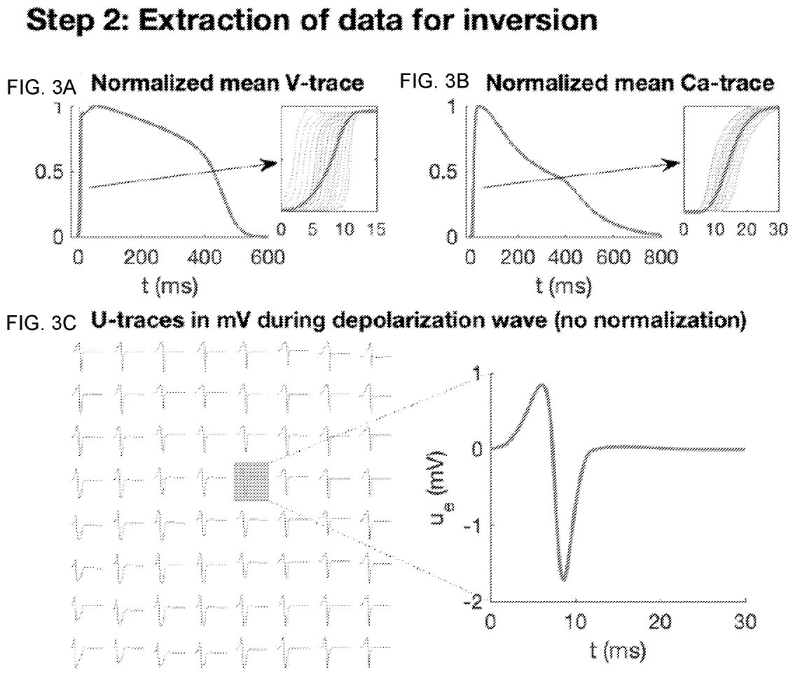

We investigate how V, Ca, and U data from a microphysiological system can be used to identify the effect of drugs. In order to generate data representing this type of recordings, we perform bidomain-base model simulations. The simulation procedure used to generate the data is illustrated in FIGS. 2-3.

As illustrated in step 1 of FIG. 2, for a given combination of A-values, we first perform bidomain-base model simulations for the duration of an entire action potential (e.g., heartbeat) and store the extracellular potential, the membrane potential, and the cytosolic calcium concentration in each grid point for each time step. A grid of four electrodes is used in this example. We then convert these recorded solutions into corresponding measurements that may be performed in microphysiological systems.

The first considered type of measurement is optical measurements of voltage-sensitive dyes. This type of measurement is mimicked in the computations by extracting the mean (or other statistical value, such as median or mode(peak value of a distribution of values)) of the membrane potential over the entire domain for each time step. Because the exact conversion factor between the pixel intensity of the optical measurements and the associated membrane potential (in mV) is not known for this type of optical measurement, embodiments can normalize the mean V-trace to values between 0 and 1. Moreover, because the time resolution of the measurement equipment for the optical measurements typically is quite limited (see e.g., [1]), we only store the membrane potential at relatively large time steps (every 10 ms). Traces representing optical measurements of the cytosolic calcium concentration can be generated in the same manner.

In addition to the optical measurements of V and Ca, the extracellular potential may also be measured in electrodes located in the microphysiological system. In the simulations, we extract this type of measurements by storing the mean extracellular potential measured at the grid points overlapping the location of the electrodes. We are primarily interested in the extracellular potential during the depolarization wave, which occurs during the upstroke. Thus, in some embodiments, we only store the extracellular potential in the beginning of the simulation (typically the first 50 ms).

FIGS. 2 and 3A-3C show a simulation procedure used to generate data for the inversions according to embodiments of the present disclosure. In Step 1, we conduct bidomain-base model simulations, recording V, Ca and U for a number of time steps. The first row of measurements corresponds to the extracellular potential (U). The second row of measurements corresponds to the transmembrane voltage V. The third row of measurements corresponds to the intracellular calcium concentration (Ca). The measurements are performed for time steps of 4 ms, 8 ms, 10, ms, 15 ms, and 300 ms.

In Step 2 in FIGS. 3A-3B, we extract V and Ca data for inversion by recording the mean V and Ca over the domain at each time step and normalizing the traces to values between 0 and 1. In addition, U is recorded (in mV) on a grid of electrodes (e.g., 8×8 electrodes from FIG. 1) at each time step during the depolarization wave.

3. Pure ODE Simplification Used in the Inversion Procedure

During the inversion procedure, we identify the λ-values associated with some considered data, generated as explained in the previous section. In this inversion procedure, we conducted simulations using a large number of different λ-values for comparison to the data. As mentioned above, the repolarization wave is known to be poorly represented by the bidomain model when applied to small collections of hiPSC-CMs. Therefore, we perform spatial simulations only for the start of the action potential. The data from this part of the simulation is used to determine the conduction velocity, the Ca transient time to peak, and the terms of the cost function related to the extracellular potential U. By performing spatial simulations only for the beginning of the action potential, we save time by avoiding a full bidomain simulation for all of the different parameter combinations. To generate the full V and Ca traces, we instead run a simple pure ODE simulations of the form (5)-(6) of the full action potential. The solution of this ODE simulation is used to compute all the terms in the cost function (see Section II.B.1), except for the ones related to U, the Ca transient time to peak, and the conduction velocity. Other embodiments can solve a partial differential equations for all the terms in the cost function.

4. Stimulation Protocol and Update of Initial Conditions

In the pure ODE simulations, the cell is stimulated by a constant stimulus current of −5 μA/cm² until v is above −40 mV. In the bidomain simulations, we stimulate the domain in the lower left corner as illustrated in FIG. 1 by a 5-ms-long constant membrane stimulus current of −40 μA/cm². Note that after each parameter change, and before any bidomain or pure ODE simulation is performed, we conduct an ODE simulation for 10 AP cycles, stimulating at 1 Hz, to update the initial conditions.

5. Numerical Methods

The numerical simulations of the bidomain model can be performed using an operator splitting procedure (see e.g., [25, 23, 26]). For each time step, we first solve the non-linear part of the system (i.e., (1) and (3) with the left-hand side of (1) set to zero), using the GRL2 method [27]. Other solving schemes may be used, e.g., Rush-Larsen (RL) solving schemes. Then, we solve the remaining linear part of the system (i.e., (1) and (2) with Iᵢₒₙ=0) using a finite difference discretization in space and a backward Euler discretization in time. Other embodiments can use other techniques, such as finite element. The discretization parameters used in the numerical simulations are given in Table 1. In the pure ODE simulations used in the inversion procedure, we apply the same GRL2 method as in the non-linear part of the bidomain simulations; and in the pure ODE simulations used to update the initial conditions after a parameter change, we use the ode15s ODE-solver in Matlab.

After the equations are solved, simulated measurements of U, V, and Ca are obtained.

B. Inversion Procedure

In this section, we will describe the inversion procedure applied to identify the effect of drugs based on V, Ca and U data in the form of adjustment factors, λ (see Section II.A.1).

As part of the inversion procedure, the input parameters (e.g., ion currents) of a model are optimized so that the output values for V, Ca and U best approximate the measured values over time (e.g., over a cycle corresponding to a heartbeat). Example ion currents include fast and late channels for sodium, potassium, and calcium. The input parameters can be measured by the adjustment factors for the ion currents.

For a given set of input parameters, the equations above are solved to provide output V, Ca and U. These output values are or are used to determine current output metrics that are used to determine a current cost value of a cost function by comparing the current output metrics to corresponding metrics from the measurements. A new set of input parameters (e.g., new ion currents) can be determined based on the cost value, e.g., whether it increased or decreased from a previous iteration. The process can repeat until one or more convergence criteria are met. Examples of terms in the cost function can include

As an example, the measurements can output data in a time series (trace) of measurements. A term in the cost function could be a sum of the differences in each data point for a given measurement trace or how quickly a given trace decreases from a maximum value. Such metrics can determine how similar a shape is of the predicted trace relative to the measured trace. Such metrics can capture key shape aspects of the data and quantitate the differences in the shapes.

1. Cost Function

In the inversion procedure, we find appropriate adjustment factors, λ, such that the solution of the model specified by λ provides output values for V, CA, and U (as output from solution of model) that are as similar as possible to the measured data. To this end, we define a cost function H(λ), measuring the difference between the measured data and the model solution (i.e., the output from the solution of the differential equations) for current values of the input parameters (e.g., ion current, as may be specific by adjustment factors, λ). An example cost function is defined as

H(λ)=Σⱼ(wⱼHⱼ(λ)²)+δ(Σᵢ|λᵢ|)², (8)

where each Hⱼ(λ) represent various differences between the data and the model solution specified by λ, and wⱼ are weights for each of these terms. In addition, δ is the weight of the regularization term (Σᵢ|λᵢ|)², which is included so that small adjustments, λ, are preferred if several choices of λ result in almost identical solutions. The individual cost function terms, Hⱼ(λ), are defined below. Example cost function terms include: (1) action potential and calcium transient durations, (2) membrane potential integral, (3) calcium transient time to peak, (4) extracellular potential, and (5) conduction velocity.

Some example terms include action potential and calcium transient durations. A number of terms in the cost function measure differences in the action potential and calcium transient durations. These terms take the form

$${H}_{APDp} ( λ ) = \frac{\vert APDp ( λ ) - {APDp}^{*} \vert}{\vert {APDp}^{*} \vert} , \qquad ( 9 )$$

$${H}_{CaDp} ( λ ) = \frac{\vert CaDp ( λ ) - {CaDp}^{*} \vert}{\vert {CaDp}^{*} \vert} , \qquad ( 10 )$$

for p=20, 30, . . . 70, 80 The APDp value is measured as the time at which V reaches a value below p % of the maximum during the repolarization phase; see FIGS. 3A-3C. The maximum can be defined for a given cycle (e.g., heartbeat cycle). APDp(λ) is the value obtained from the solution of the model given by the parameter vector λ, whereas APDp* is the value obtained from the data. The calcium transient durations, CaDp, are defined just like the action potential durations. Note also that the notation of a * marking the data values is used for all the terms in the cost function (see below). Another example term is the area under the measurement curve (trace) when V or Ca reach p % below the maximum, which can be denoted as Int30, Int50, etc, as described more below.

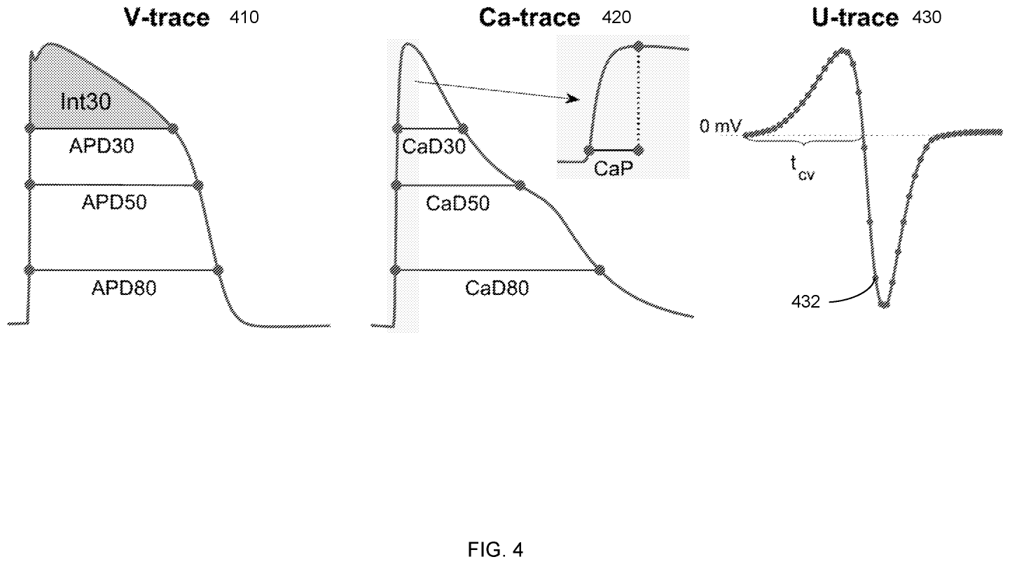

FIG. 4 illustrates some of the properties included in the cost function (8) according to embodiments of the present disclosure. The measurement traces of V, Ca, and U over time (horizontal axis) is shown. From the V-trace 410, we include a number of APD-values (see (9)), and the integral of V above APD30 (see (11)). From the Ca-trace 420, we include a number of CaD-values (see (10)) in addition to the Ca transient time to peak, CaP (see (13)). The transient time to peak can correspond to the time for the Ca-trace 420 to go from a transient level before an input electrical signal causing contraction to the maximum value. Every time the cell beats, the cell membrane depolarizes and the voltage trace increases, causing the cell to release calcium, which is what causes the muscle to twitch resulting in a heartbeat. A global measure of Ca and V from the experiments is used, even though they exist discretely everywhere in the simulation. Other implementations can use measured values at different spatial locations.

From the U data 430, we include the value at each time point and each electrode (see (14)), and the conduction velocity is computed using the time points in which U crosses 0 mV after the peak in different electrodes (see (15) and FIG. 1). Given that U is measured at each electrode, another metric can be the time t_{cv} for the U data 430 to go back to zero. Another metric uses the measurements at each of the time points (shown as separate dots 432; a sum of the square of the differences (or absolute value) between the model output and measured values can be summed.

Another example term is a membrane potential integral. The cost function also includes a term of the form

$${H}_{Int 30} ( λ ) = \frac{\vert Int 30 ( λ ) - Int {30}^{*} \vert}{\vert Int {30}^{*} \vert} , \qquad ( 11 )$$

where Int30 is defined as

Int30=∫ₜ_{₁}^{t}^{₂}[v−v(t₁)]dt (12)

and v is the membrane potential. The values t₁ and t₂ are here time points corresponding to when V is 30% below the maximum value during the depolarization and repolarization phases of the action potential (see FIG. 4).

Another example term is a calcium transient time to peak. The typical time resolution of the V and Ca data (10 ms) is too coarse to be able to detect changes in the upstroke velocity of the action potential. However, the upstroke of the calcium transient is slower, and therefore, changes in the upstroke of the calcium transient may be detected. To measure this upstroke velocity, we include a term of the form

$${H}_{t , Ca} ( λ ) = \frac{\vert CaP ( λ ) - {CaP}^{*} \vert}{\vert {CaP}^{*} \vert} , \qquad ( 13 )$$

where CaP is the time from Ca is 10% above its minimum value until it reaches its maximum (see FIG. 3).

As mentioned above, a bidomain simulation can be used instead of a pure ODE simulation to compute an estimate of CaP(λ) (and CaP* in the case of simulated data). This bidomain simulation can be conducted for a rather short time interval (typically 50 ms) and record Ca in all the grid points, e.g., the 4×4 grid of electrodes, which can be used for a finite difference solver. Then, we extract the grid points that have reached their peak Ca concentration during this simulation, and set up a normalized mean Ca-trace for these grid points. The value of CaP is then computed from this normalized mean Ca-trace. When the data is generated, we use the same procedure of considering the solution for only the first part of the simulation (e.g., 50 ms) when we compute CaP*. In various embodiments, the measured values for Ca at the grid points can be averaged to determine to the global measurement of the Ca trace in the experiment or the individual measurements may be used.

Another example term is an extracellular potential. In order to detect changes in U measured at the electrodes, we include a cost function term of the form

$${H}_{U} ( λ ) = \frac{{\sum }_{k} {‖ {u}_{e}^{k} ( λ ) - {u}_{e}^{k , *} ‖}_{2}}{{\sum }_{k} {‖ {u}_{e}^{k , *} ‖}_{2}} , \qquad ( 14 )$$

where uₑ^{k} is a vector denoting U (in mV) in electrode k for each of the recorded time steps. In this example, an L1 norm is used, but an L2 norm could also be used.

Another example term is a conduction velocity. In addition, the cost function includes a term for the conduction velocity of the form

$${H}_{c v} ( λ ) = \frac{\vert cv ( λ ) - c {v}^{*} \vert}{\vert {cv}^{*} \vert} , \qquad ( 15 )$$

where cv denotes the conduction velocity (in cm/s) computed from U recorded in the electrodes. More specifically, the conduction velocity is computed as the distance between the center of the two electrodes marked as a and b in FIG. 1 divided by the time between U crosses 0 mV after the peak in these two electrodes (see FIG. 4). The two electrodes used could be any two electrodes in the simulation.

Cost function weights can also be used. In the applications of the inversion procedure reported below, we have used the weight wⱼ=1 for each of the cost function terms, except for H_{APD80} and H_{CaD80}, which are given the weight of 5. In addition, we use the value δ=0.01 for the regularization term.

2. Continuation-Based Optimization

In order to find the optimal λ-values minimizing the cost function H defined in (8), embodiments can apply a continuation-based minimization procedure. This approach is described in detail in [2]. In short, embodiments can attempt to find the optimal λ-values (i.e., specify input ion currents) by gradually moving from a known solution λ⁰=0 to the final A fitting the data as best possible. By known solution, we mean a known solution to equations (1)-(3) to obtain the output values for U, V, and Ca as opposed to the known solution for the optimized input parameters. Such a known solution can be a base model or be a solution from a control model, when performing the inversion for the drug-treated model.

To this end, we introduce a parameter 0 that is gradually increased from 0 to 1, and define an intermediate cost function

$$\overline{H} ( λ , θ ) = {\sum }_{j} ( {w}_{j} {{\overline{H}}_{j} ( λ , θ )}^{2} ) + \frac{δ}{max ( θ , ξ )} {( {\sum }_{i} \vert {λ}_{i} \vert )}^{2} , \qquad ( 16 )$$

where H̅ⱼ(λ, θ) are adjusted versions of each of the cost function terms defined above and is some small number (e.g., 10⁻¹⁰). In general, H̅ⱼ(λ, θ) is defined as

$${\overline{H}}_{j} ( λ , θ ) = \frac{\vert {R}_{j} ( λ ) - {R}_{j}^{*} ( θ ) \vert}{\vert {R}_{j}^{*} ( θ ) \vert} , \qquad ( 17 )$$

where Rⱼ is some property of the solution, for example an action potential duration. Moreover, Rⱼ(λ) is the property computed in the solution of the model defined by λ, and R*ⱼ(θ) is defined as

Rⱼ*(θ)=(1−θ)Rⱼ(λ₀)+θRⱼ*, (18)

where Rⱼ(λ₀) is the property computed from the model solution defined by λ⁰=0, and Rⱼ* is the property computed from the data we are trying to invert.

Considering the cost function terms, Hⱼ, defined above, all the terms may be defined on the form (17) except for the term H_{U} defined in (14). For this term, we instead define

H̅_{U}(λ,θ)=(1−θ)H_{U}⁰+θH_{U}(λ), (19)

where H_{U}⁰ is defined by (14) with uₑ^{k,}* replaced by the model solution uₑ^{k}(λ₀), and H_{U}(λ) is defined by (14).

From the definitions (16)-(19), we observe that for θ=0, Rⱼ*=Rⱼ(λ₀) and H̅ⱼ(λ₀, 0)=0, implying that the known solution λ=λ₀ minimize (16) for θ=0. Furthermore, for θ=1, Rⱼ*(1)=Rⱼ* defined by the data, H̅ⱼ(λ, 1)=Hⱼ(λ), and H̅(λ, 1)=H(λ). Consequently, for θ=1, the minimum of H defined in (16) is the same as the minimum of H defined in (8).

The advantage of the continuation algorithm is that we can move from the known optimal value λ₀ to the final optimal value A gradually in a number of iterations, and that we for each iteration can assume that the new estimated A is in the vicinity of the optimal λ from the previous iteration. In the applications of the inversion procedure reported below, we use four θ-iterations in the continuation algorithm (θ=¼, θ=½, θ=¾ and θ=1). In the first three iterations, m, we draw 63 random initial guesses of λ(θₘ) in the vicinity of λ(θₘ₋₁), and in the last iteration, we draw 126 initial guesses. More specifically, the initial guesses for λᵢ(θₘ) are drawn from [λᵢ(θₘ₋₁)−0.2, λᵢ(θₘ₋₁)+0.2], where λᵢ(θ₀) is defined to be 0. From these initial guesses we minimize the cost function H̅(λ, θ) using the Nelder-Mead algorithm [28]. In the first three θ-iterations, we use 10 iterations of the Nelder-Mead algorithm for each initial guess, and in the last θ-iteration, we use 25 iterations.

In various embodiments, the cost function is iteratively minimized. In some embodiments, between 2 and 100 (e.g., 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100) iterations are used. In some embodiments, more than 100 iterations are used.

In various embodiments, during each iteration, for each parameter that is being updated, 1 or more values for the parameter are selected (e.g., randomly selected). In particular embodiments, the one or more values for each parameter are selected from within an interval of allowed values. In some instances, between about 1,000 and about 5,000 (e.g., about 1,000, 1,100, 1,200, 1,300, 1,400, 1,500, 1,600, 1,700, 1,800, 1,900, 2,000, 2,100, 2,200, 2,300, 2,400, 2,500, 2,600, 2,700, 2,800, 2,900, 3,000, 3,100, 3,200, 3,300, 3,400, 3,500, 3,600, 3,700, 3,800, 3,900, 4,000, 4,100, 4,200, 4,300, 4,400, 4,500, 4,600, 4,700, 4,800, 4,900, or 5,000) values for each parameter being updated are selected (e.g., randomly selected). In some embodiments, more than 5,000 values are selected. When an interval of allowed values (i.e. for a parameter that is being updated in a given iteration) is used, in some embodiments, the length or size of the interval is decreased with each iteration. In some embodiments, the length or size of the interval is decreased with each iteration by about 90%, 80%, 70%, 60%, 50%, 40%, 30%, 20%, or 10%.

C. Adjustment of Extracellular Concentrations

In order to better distinguish between different ion channel blockers, we consider the drug effects under different extracellular conditions. In particular, we vary the extracellular calcium concentration by introducing a number of known adjustment factors γ_{Ca} such that

[Ca²⁺]ₑ=(1+γ_{Ca})[Ca²⁺]ₑ*, (20)

where [Ca²⁺]ₑ is the extracellular calcium concentration and [Ca²⁺]ₑ* is the default extracellular calcium concentration reported in Table 2.

In the inversions reported below, we use the two values γ_{Ca,1}=0 and γ_{Ca,2}=0.25. We generate V, Ca and U data using the approach described in Section II.A.2 for both of these values of γ_{Ca}. Furthermore, for a given choice of λ in the inversion procedure, we compute a version of the solutions for each of these Y_{Ca}-values, and define the cost function

H(λ)=Σ_{q}Σⱼ(wⱼHⱼ(wⱼHⱼ(λ,γ_{Ca,q})²))+δ(Σᵢ|λᵢ|)², (21)

where q counts the different extracellular conditions and Hⱼ(λ, γ_{Ca,q}) represents the cost function terms computed using the extracellular calcium concentration defined by γ_{Ca,q}.

### III. RESULTS

In this section, we report the results of some applications of the inversion procedure defined above. First, we investigate the sensitivity of the V, Ca and U data in response to perturbations of the I_{Kr}, I_{CaL} and I_{Na} currents, and how this sensitivity may be increased when data for several different extracellular calcium concentrations are included. Next, we show some examples of extracellular repolarization waves, and explain why we have chosen to only consider the extracellular depolarization waves in the inversion procedure. Afterwards, we show some examples of how bidomain-base model simulations are able to reproduce measured drug induced effects on the conduction velocity, illustrating that including U data in the inversion procedure improves the identifiability of drug effects on I_{Na}. Finally, we test the inversion procedure by using it to identify drug effects for a number of simulated drugs and investigate how the accuracy of the inversion is affected when noise is included in the simulated data.

A. Sensitivity of currents

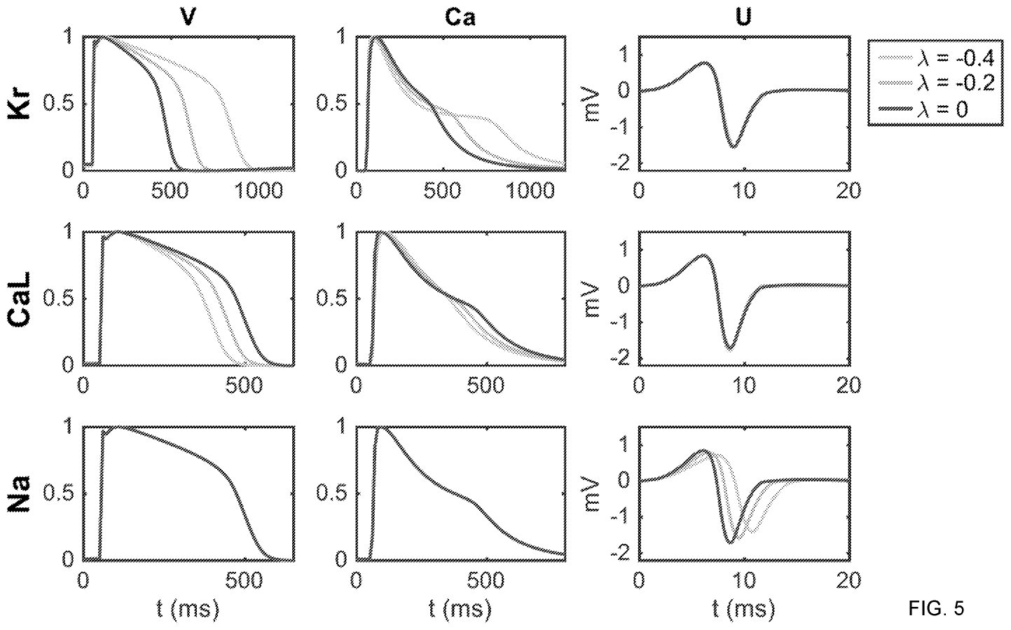

FIG. 5 shows an effect of perturbing I_{Kr}, I_{CaL} and I_{Na} on the V, Ca and U data according to embodiments of the present disclosure. For the U data, we show the extracellular potential U in the electrode marked as c in FIG. 1, and the time scale is zoomed in on the first 20 ms of the simulation. Each row corresponds to perturbing a different ion channel current, specifically, I_{CaL} and I_{Na}.

In FIG. 5, we investigate the effect on the simulated V, Ca and U data of perturbing the I_{Kr}, I_{CaL} and I_{Na} currents. We compute the data for the default base model (λ=0) and for two perturbations λᵢ=−0.2 and λᵢ=−0.4 for each of the currents i=Kr, CaL, Na, corresponding to 20% and 40% block of the currents, respectively (see Section II.A.1). Note that in the upper panel (investigating block of I_{Kr}), we have used a pacing frequency of 0.5 Hz instead of 1 Hz in order to allow for increased action potential durations resulting from block of I_{Kr}.

We observe that block of the I_{Kr} current results in increased action potential and calcium transient durations, whereas block of I_{CaL} results in decreased action potential and calcium transient durations. Moreover, no effects are visible in the U data resulting from block of I_{Kr} or I_{CaL} (recall that the U data is only considered for the first 20 ms). For block of the I_{Na} current, on the other hand, no effects on the action potential and calcium transient durations are visible, but there are clearly visible effects on the U data. Specifically, the amplitude of U is decreased and the timing of the peaks is delayed in response to block of the I_{Na} current. FIG. 5 shows expected result from blocking a number of specified currents, and how they should effect measurements of V, Ca and extracellular potential, thereby confirming an accuracy of the technique.

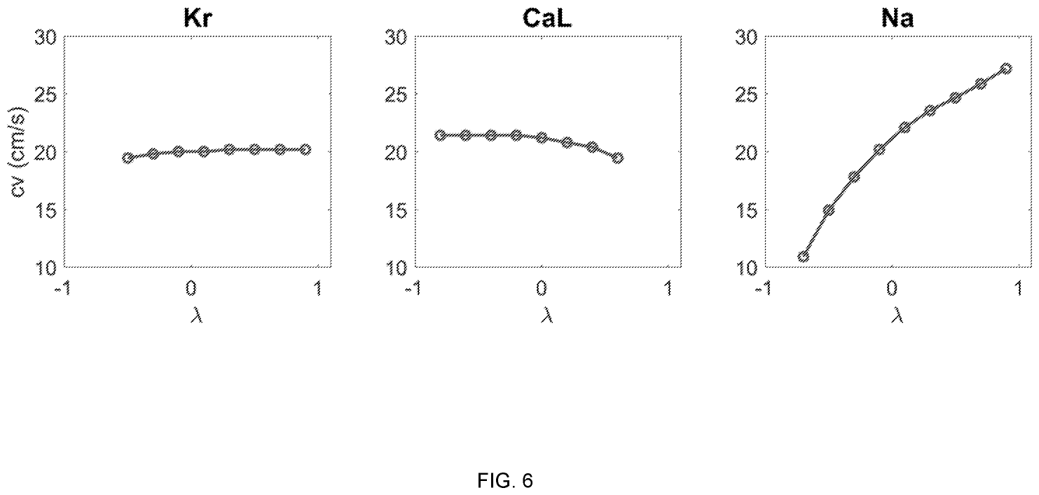

FIG. 6 shows an effect of perturbing I_{Kr}, I_{CaL} and I_{Na} on the computed conduction velocity according to embodiments of the present disclosure. The conduction velocities are computed from the U data as explained in Section II.B.1.

In FIG. 6, we report the conduction velocities computed from the U data in response to perturbations of the three currents. We observe that for perturbations of I_{Kr} and I_{CaL}, the effects on the conduction velocity are quite small, whereas block of I_{Na} results in a considerably decreased conduction velocity.

B. Adjustment of Extracellular Concentrations

Because the effect on the V, Ca and U data of blocking I_{Kr} is almost exactly the opposite of the effect of blocking I_{CaL} (see FIG. 5), we expect that determining the correct combination of blocking adjustment factors (k) for these two currents will be difficult. Therefore, it could be useful to consider drug effects under different extracellular conditions in order to increase the chance of identifying the correct block for the two currents. For example, local minima and non-unique solutions could exist, so there may be ways of changing the dynamics of the system to differentiate the correct result.

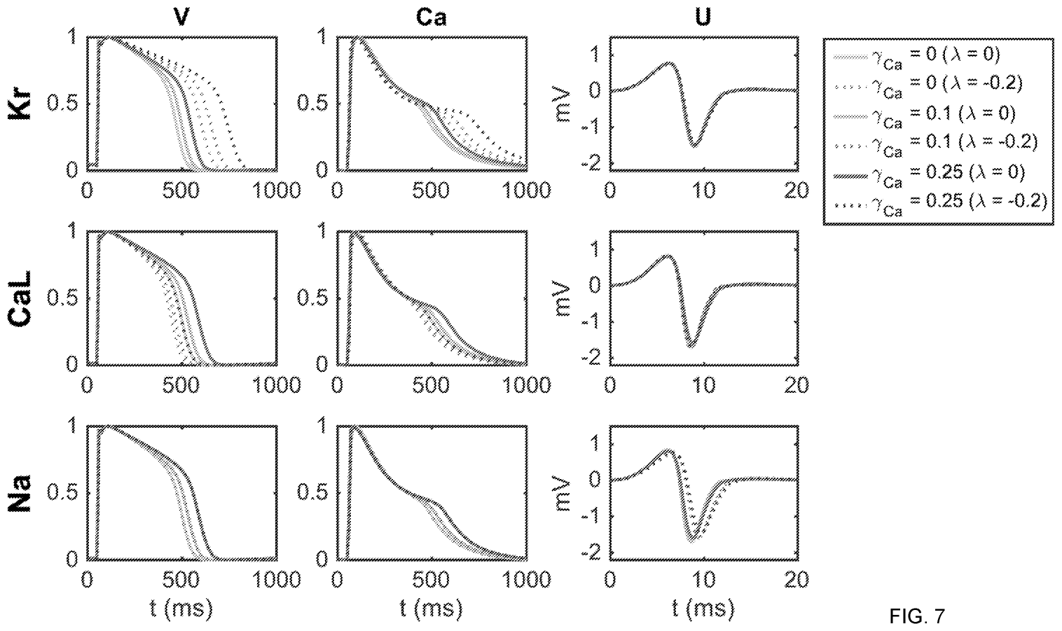

FIG. 7 shows the effect of block of the I_{Kr}, I_{CaL} and I_{Na} currents for three different extracellular calcium concentrations according to embodiments of the present disclosure. The extracellular calcium concentrations can be controlled experimentally. FIG. 7 shows similar plots as FIG. 6, but with additional lines for different extracellular calcium concentrations. In particular, FIG. 7 shows an effect of perturbing I_{Kr}, I_{CaL} and I_{Na} on the V, Ca and U data for three different adjustments, γ_{Ca}, of the extracellular calcium concentration (see Section II.C). For the U data, we show the extracellular potential in the electrode marked as c in FIG. 1, and the time scale is zoomed in on the first 20 ms of the simulation.

We consider the default extracellular calcium concentration specified in Table 2 (γ_{Ca}=0), a 10% increased concentration (γ_{Ca}=0.1), and a 25% increased concentration (γ_{Ca}=0.25). The solid lines show the default solutions for each of these extracellular environments, and we observe that the action potential and calcium transient durations are increased as the extracellular calcium concentration is increased. Furthermore, the dotted lines show the solutions corresponding to 20% block of the considered currents. For all the considered U data and for the V and Ca data for block of I_{Na}, the different extracellular calcium concentrations do not seem to have a significant effect. However, for the V and Ca data for block of I_{Kr} and I_{CaL}, we observe that the effect of the block varies for the different concentrations. For example, the effect of block of I_{Kr} is more prominent for an increased extracellular calcium concentration.

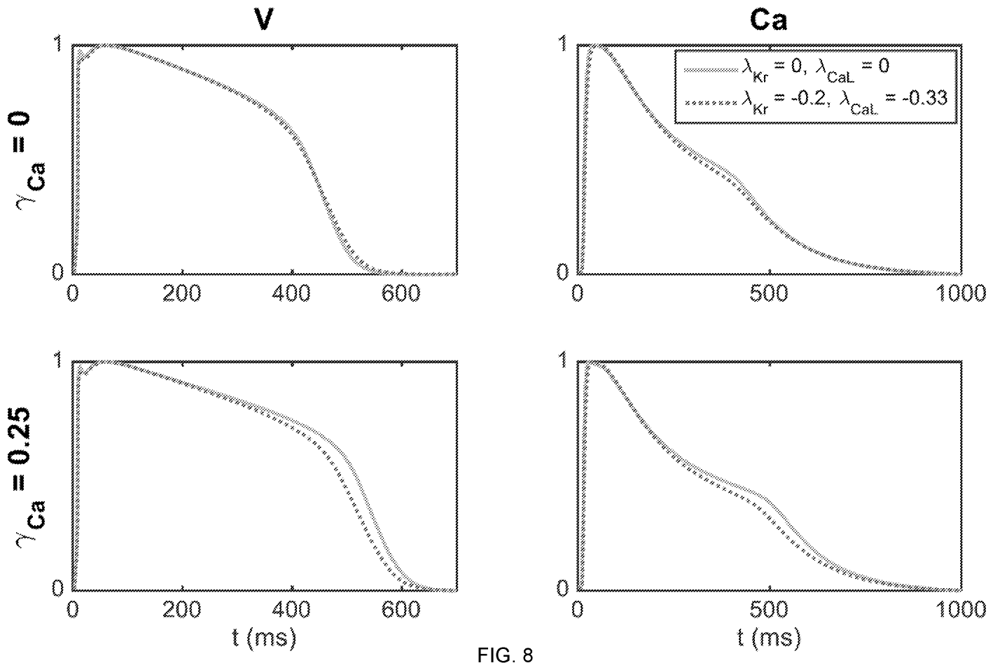

FIG. 8 illustrates how including an extra extracellular calcium concentration adjustment may improve the identifiability of I_{Kr} and I_{CaL} according to embodiments of the present disclosure. In the upper panels, we compare V and Ca for two different block combinations for the default extracellular calcium concentration (γ_{Ca}=0). In the lower panels, we consider the solutions for a 25% increased extracellular calcium concentration (γ_{Ca}=0.25). For γ_{Ca}=0, the two solutions are very similar, but the difference is increased for γ_{Ca}=0.25. This indicates that testing at multiple controlled extracellular concentrations may improve inversion uniqueness and help resolve the correct channel block.

In this example, including an additional extracellular calcium concentration could help identify the correct block of I_{Kr} and I_{CaL}. In the upper panel, we use the default extracellular calcium concentration of Table 2. The solid line shows V and Ca for the default base model, and the dotted line shows the solution for a case with 20% block of I_{Kr} and 33% block of I_{CaL}. We observe that V and Ca look very similar in these two cases. As a result, the inversion procedure might mistake the case of (λ_{Kr}=−0.2, λ_{CaL}=−0.33) to the case with no block. In the lower panel, however, we compare the two solutions for the case with an increased extracellular calcium concentration, and in this case, the difference between the solutions is more prominent. Consequently, the inversion procedure can more easily distinguish between the two cases when the solutions for both extracellular calcium concentrations are included.

C. Estimating the Action Potential Duration

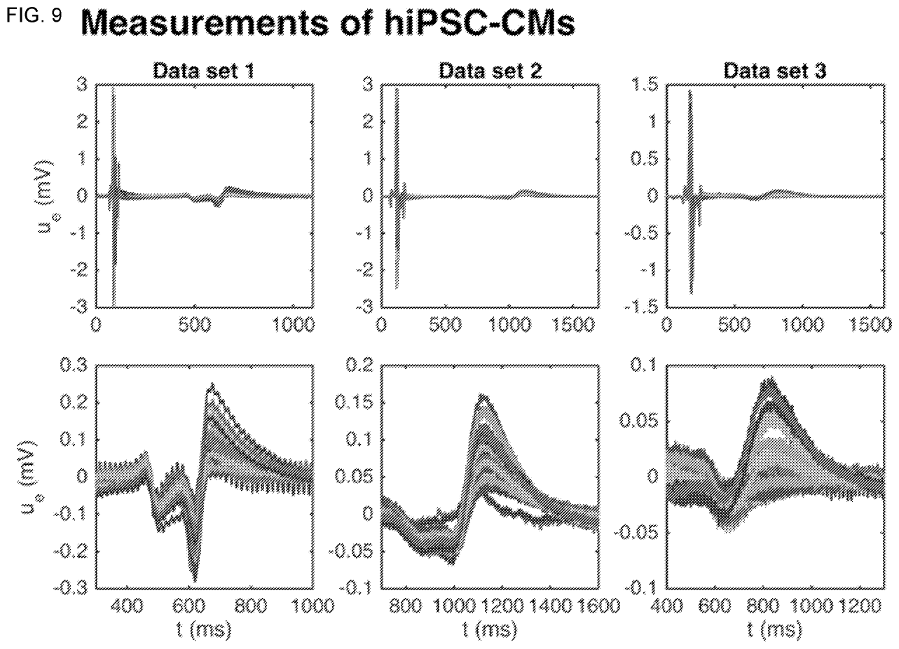

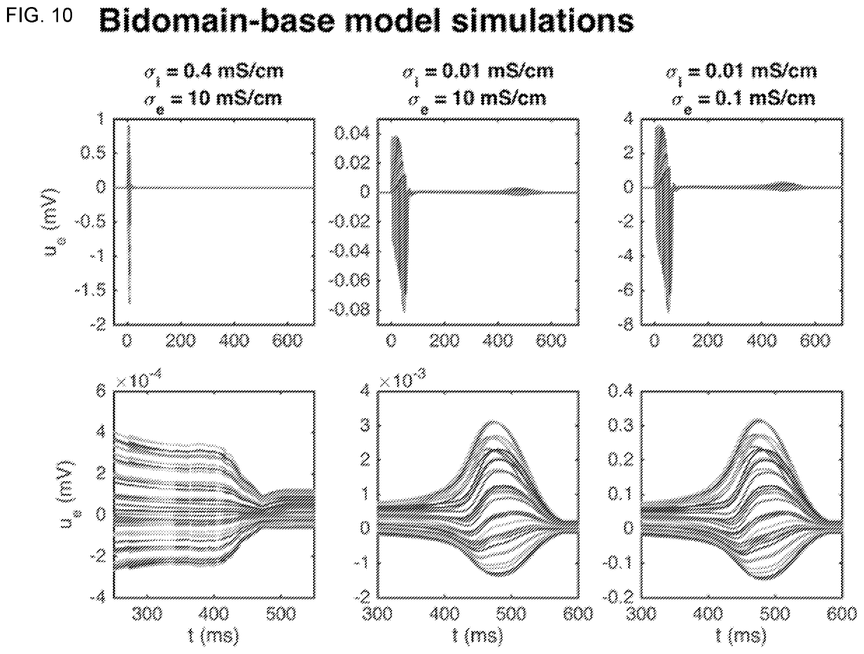

FIGS. 9 and 10 shows a repolarization wave in hiPSC-CM data and bidomain-base model simulations according to embodiments of the present disclosure. The upper panels in FIG. 9 shows measurements of U during an action potential for three different data sets. The traces for the 64 electrodes are overlaid in the plots. In the second panel of FIG. 9, we zoom in on the repolarization wave. The third panel (first panel in FIG. 10) shows U in three bidomain-base model simulations, and the bottom panel in FIG. 10 shows the corresponding repolarization waves.

More specifically, FIG. 9 shows measurements of the extracellular potential in 64 electrodes recorded for collections of hiPSC-CMs. In the upper panels, we observe that there is some early activity, corresponding to the time of the upstroke of the action potential, in addition to a weaker signal after some hundred milliseconds. This weaker signal occurs at the time of repolarization of the action potential and is therefore referred to as the repolarization wave. In the lower panel, we zoom in on this repolarization wave, and we observe that the extracellular potential reaches a magnitude of up to about 0.1-0.2 mV in this period.

Bidomain simulations of small collections of hiPSC-CMs can tend to give rise to very weak or even non-existing repolarization waves. Indeed, in the leftmost panels of FIG. 10, we plot the extracellular potential in a bidomain-base model simulation using the default parameter values of Table 1; we observe that the magnitude of the wave in this case is very small, and is completely invisible in the upper plot over the entire action potential duration. If we decrease the intracellular conductivity, σᵢ, on the other hand, a repolarization wave is visible, but the entire extracellular signal is quite weak (see the center panels of FIG. 10). However, if the extracellular conductivity, σₑ, is decreased as well, the size of both the depolarization wave and the repolarization wave are quite similar to the recorded data (see the rightmost panels of FIG. 10).

In theory, the repolarization waves observed in the data and simulations could be used to estimate the action potential duration in the inversion procedure. However, as observed in the lower panels of FIG. 10, the repolarization waves are quite smooth, and it is not clear which time points represent different degrees of repolarization. For the V-traces on the other hand (see e.g., FIG. 2), it is straightforward to define accurate measures of different degrees of repolarization in the form of APD-values (see Section II.B.1 and FIG. 5). Therefore, we use the optical measurements of V to define the action potential durations and use the U data for information regarding the depolarization wave. Thus, in some embodiments, only a certain portion of U during a cycle is used in the cost function, where the specific parts of U to be used is based on measurements of V.

The parameters used in the simulations are given in Table 1, except for σᵢ and σₑ which are reported in the plot titles.

| | | CV simulation | CV simulation |
|---|---|---|---|
| | | (IC50_{Na} = 2.2 μM, | (IC50 _{Na} = 0.2 μM, |
| Dose | CV data | h_{Na} = 1) | h_{Na} = 0.4) |
| 0 μM | 23.7 cm/s | 23.6 cm/s | 23.7 cm/s |
| 1 μM | 17.9 cm/s | 20.0 cm/s | 14.2 cm/s |
| 2.5 μM | 11.7 cm/s | 16.7 cm/s | 12.0 cm/s |
| 10 μM | 9.3 cm/s | 9.2 cm/s | 8.9 cm/s |

Table 3 shows an effect of the drug Flecainide on the conduction velocity computed from the extracellular potential. The left panel reports values based on measurements of hiPSC-CMs and the center and right panels report values computed in bidomain-base model simulations with IC50_{CaL}=9 μM, IC50_{NaL}=47 μM, IC50_{Kr}=1.9 μM, and hᵢ=1, for i=CaL, NaL, and Kr (see (22)). The drug effect on I_{Na} is set to IC50_{Na}=2.2 μM, h_{Na}=1 in the center column and IC50_{Na}=0.2 μM, h_{Na}=0.4 in the right column. The parameters used in the simulations are given in Tables 1 and 2, and the default g_{Na} value is increased by 45% to match the conduction velocity in the control case.

D. Estimating Drug Induced Effects on the Conduction Velocity

One of the advantages of including measurements of the extracellular potential in addition to optical measurements of V and Ca in the inversion procedure is that the extracellular measurements can be used to estimate the conduction velocity of the cell collection. This information could be useful for determining drug effects on the I_{Na} current (see FIG. 6). The left column of Table 3 reports the conduction velocity computed from measurements of the extracellular potential in a collection of hiPSC-CMs exposed to different doses of the drug Flecainide. We observe that as the drug dose is increased, the conduction velocity is decreased. This could indicate that the drug blocks the I_{Na} current, which has also been found in previous studies [5, 7]. The results below show that the conduction velocity can be accurately determined.

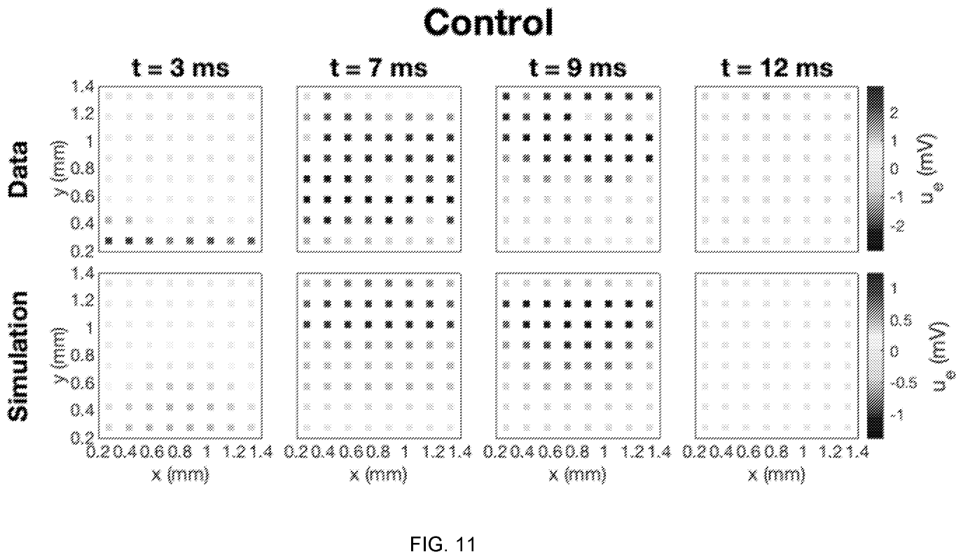

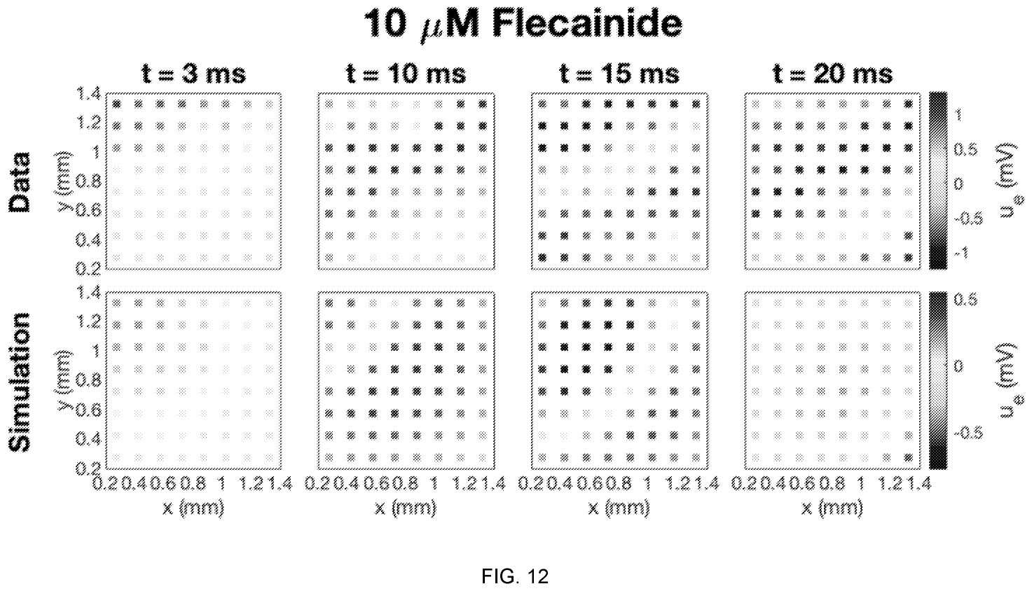

FIGS. 11 and 12 show measured and simulated U data in 64 electrodes for the control case and for a dose of 10 μM Flecainide at different points in time according to embodiments of the present disclosure. The was modeled using IC50_{CaL}=9 μM, IC50_{NaL}=47 μM, IC50_{Kr}=1.9 μM, IC50_{Na}=0.2 μM, hᵢ=1 for i=CaL, NaL, and Kr, and h_{Na}=0.4. The parameters used in the simulations are given in Tables 1 and 2, and the default g_{Na} value is increased by 45%. In the simulations, we have adjusted the stimulation location to correspond to the propagation direction observed in the measured data. In the control case, the wave moves in the y-direction from the lower part of the domain to the upper part, and in the drug case, the wave moves from the upper left corner to the lower right corner of the domain. In both the data and the simulations, we observe that the extracellular depolarization wave moves more slowly across the domain for 10 μM Flecainide than in the control case, consistent with the reduced conduction velocities observed in Table 3.

In the paper [2], we estimated IC50-values for I_{CaL}, I_{NaL} and I_{Kr} based on optical measurements of V and Ca of hiPSC-CMs exposed to Flecainide, but we were not able to estimate the effect on I_{Na} due to the low time resolution for the optical measurements. The IC50-values were estimated to IC50_{CaL}=9 μM, IC50_{NaL}=47 μM, and IC50_{Kr}=1.9 μM, where the conductance of each current was scaled according to

$${g}_{i}^{d} = ( \frac{1}{1 + {( \frac{D}{IC {50}_{i}} )}^{{h}_{i}}} ) \cdot {g}_{i}^{c} , fori = CaL , NaL , Kr , \qquad ( 22 )$$

where gᵢ^{d} is the conductance of current i in presence of the drug dose D and gᵢ^{c} is the conductance in the control case with no drug present. Furthermore, hₗ is the so-called Hill coefficient, assumed to be 1 in [2]. Incorporating these IC50-values in addition to some estimated IC50-values for I_{Na} in bidomain-base model simulations, we obtain the conduction velocities reported in the center and right columns of Table 3. In the center column we use IC50_{Na}=2.2 μM and h_{Na}=1, and in the right column we use IC50_{Na}=0.2 μM and h_{Na}=0.4.

Note that since high doses of Flecainide result in increased action potential durations (see e.g., [2]), we have used a pacing frequency of 0.5 Hz instead of 1 Hz when updating the initial conditions for these simulations. In addition, to match the conduction velocity in the control case, the default value of g_{Na} value of Table 2 is increased by 45%. We observe that the bidomain-base model simulations are able to roughly reproduce the drug induced reduction in conduction velocity observed in the measurements, indicating that a comparison of measured and simulated conduction velocities could help identify drug effects on I_{Na}.

E. Inversion of Simulated Drugs

In order to investigate how well the inversion procedure outlined above is able to identify the effect of drugs on I_{Kr}, I_{CaL} and I_{Na}, we generate data for twelve drugs whose effect on I_{Kr}, I_{CaL} and I_{Na} was investigated in [7]. The block percentages used to generate the data are based on the block percentages for drug concentrations corresponding to three times the free plasma Cₘₐₓ reported in [7]. However, large block percentages for I_{Kr} have been reduced in order to obtain reasonable V and Ca traces. In the inversions, we use two extracellular calcium concentrations, as explained in Section II.C. Furthermore, we save U data and information about the Ca peak time extracted from the first 50 ms of the bidomain simulations for all the drugs, except for the drug Diltiazem. For Diltiazem, we had to increase the bidomain simulation time to 100 ms in order to ensure that some of the grid points in the domain had reached their peak calcium concentration to compute the Ca peak time (see Section II.B.1).

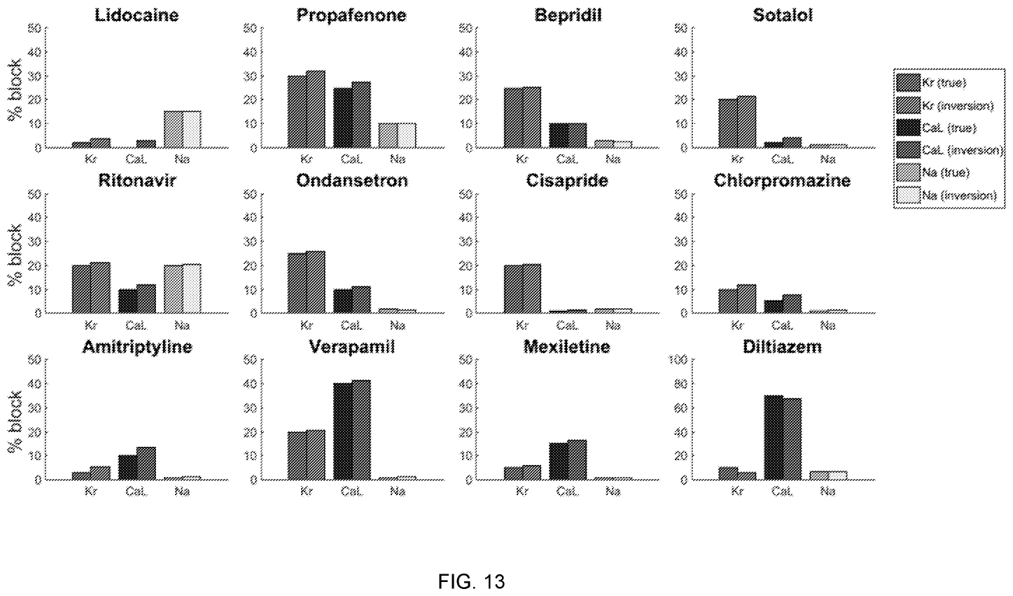

FIG. 13 shows a result of the inversion procedure applied to simulated data of twelve drugs, based on the block percentages corresponding to three times the free plasma Cₘₐₓ reported in [7] according to embodiments of the present disclosure. Accordingly, we compare the block percentages estimated by the inversion procedure to those used to generate the data for the twelve drugs. We observe that for all the considered drugs, the inversion procedure is able to identify the block of the three currents quite accurately.

F. Effect of Noise

In FIG. 13, we considered how well the inversion procedure was able to identify drugs based on data generated by bidomain-base model simulations, and we observed that the inversion procedure was able to identify the correct channel blocking quite accurately. However, when the V, Ca and U data are recorded from real measurements from microphysiological systems, the data will include some noise. In order to investigate how well the inversion procedure is able to identify the effect of drugs from data including noise, we include 5% noise in the V data, 3% noise in the Ca data and 1% noise in the U data and repeat the inversions shown in FIG. 13. The noise is added by drawing a random number between −p·A and p·A for each point in time, where A is the difference between the maximum and minimum value of the considered data and p is the noise percentage. This random number is then added to the V, Ca and U traces.

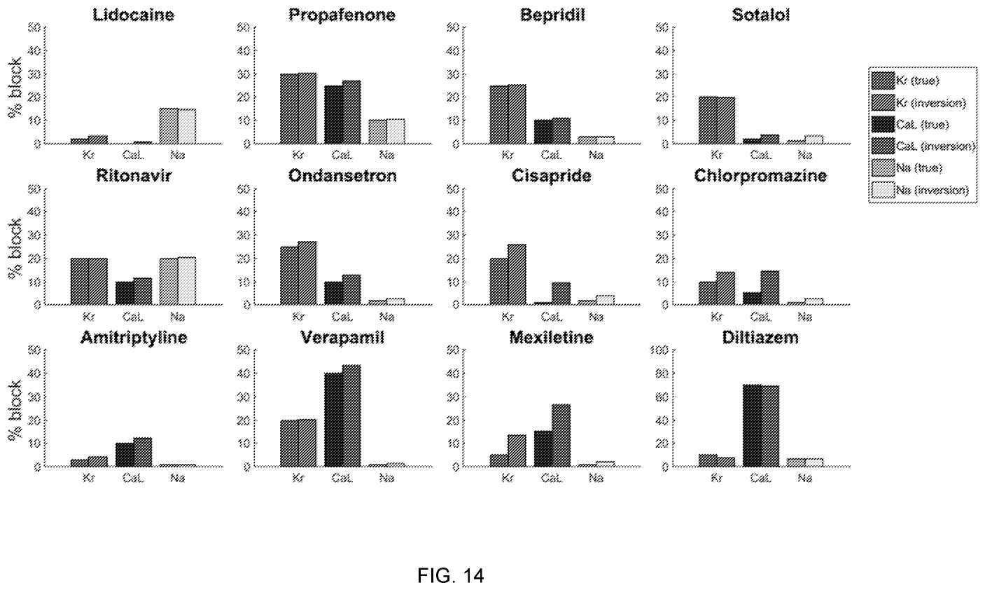

FIG. 14 shows a result of the inversion procedure applied to simulated data of the twelve drugs of FIG. 10 according to embodiments of the present disclosure. We have here included 5% noise in the V data, 3% noise in the Ca data and 1% noise in the U data.

The result of the inversions with noise included in the data is given in FIG. 14. We observe that the inversion procedure is able to estimate the block percentage quite accurately in most cases, but that the accuracy is reduced compared to the case with no noise in FIG. 13.

### IV. DISCUSSION

A. Sensitivity of Parameters is Necessary for Identification

A generic problem in mathematical models of physiology is to determine the parameters included in the model. For models of the action potential of excitable cells, there is a large number of parameters that needs to be determined in order to use the model; see e.g., [29, 30, 31, 32]. I_{f} a model is insensitive to changes in a specific parameter, that parameter is impossible to determine by comparing experimental results and results of numerical simulations. Contrarily, if the model is sensitive to a parameter, numerical experiments can be used to match the model to the data by fine-tuning the parameter. In FIG. 5 above, we saw that the membrane potential V is clearly sensitive to changes in the I_{Kr} and I_{CaL} currents. The action potential upstroke (which occurs when the cell starts depolarizing) is also sensitive to changes in the I_{Na} current, but since the upstroke is very fast, the sensitivity is difficult to see in the plot of the entire AP. Since optical measurements have a relatively coarse time resolution, we have been unable to identify the strength of the sodium current based solely on voltage and calcium traces. However, we note from FIG. 5 that the extracellular potential is indeed sensitive to changes in the sodium current. This sensitivity also carries over to sensitivity of the conduction velocity (CV) with respect to changes in the sodium current, and this sensitivity is utilized in the cost function; see (15).

B. The Conduction Velocity is Governed by the Sodium Current

The conduction of the electrochemical signal through the cardiac muscle is essential for the functioning of the heart. Surprisingly, the conduction process is still not completely understood; see, e.g., [33] for a review of the development of the understanding of cardiac conduction. Globally (mm scale), cardiac conduction is usually modeled using the bidomain or monodomain models (see e.g., [17, 22, 23]), but locally (μm scale) more detailed models are used; see e.g., [34, 35, 36]. The microphysiological system is somewhere in between these scales (the size is about 1.2 mm×1.2 mm). Some embodiments can use the detailed EMI model introduced for cardiac conduction in [37]. As described above, the bidomain model can be used to consider how the CV depends on changing the different ion currents. The EMI and bidomain models are two example models that may be used with embodiments of the disclosure.

In FIG. 6, we observe that the conduction velocity (CV) is very sensitive to changes of the I_{Na} current, but almost insensitive to changes in the I_{Kr} and I_{CaL} currents. This means that we can use the measurements of U to identify the I_{Na} current first and then only look for the I_{Kr} and I_{CaL} currents based on the V- and Ca-traces.

C. Improving Visibility of Parameters by Changing the Extracellular Concentrations

In using experimental data to determine parameters of a computational model, it is desirable to use protocols designed to highlight the effect of different parameters. For instance, it is argued in [38] that random stimulation protocols can improve visibility of the parameters, and this is confirmed in [32]. Another experimental parameter that can be changed is the ionic concentrations of the extracellular environment. In FIG. 7 and FIG. 8, we show that using two different extracellular calcium concentrations (as determined experimentally) can improve identifiability of the model parameters. One particular important effect of this is that multiple extracellular calcium concentration can aid in distinguishing changes of the I_{Kr} and I_{CaL} currents. Since blocking I_{Kr} has more or less the same effect as increasing I_{CaL}, it is difficult to distinguish these effects in measurements of the AP. But it is apparent from FIG. 8 that the level of blocking changes with the extracellular calcium concentration, and this can be used to distinguish between different blocking combinations for these two currents.

D. Inversion of Simulated Drugs

Simulated data is often used as a proxy for real data when the real data are cumbersome to obtain. Here, we have used real data for U—both for the case of no drug and when various doses of Flecainide have been applied. Furthermore, the base model used to represent the membrane dynamics has been parameterized using real data; see [2, 1]. However, in order to study inversion for the range of data provided in the CIPA report [7], we have used simulated data. One advantage of this is that we can get data to any desired accuracy and that we can control the level of noise inevitably introduced in the measurements. The disadvantage is clearly the reduced realism of the data and it is a priority of future work to use combined V, Ca, and U data to do inversion of both well characterized and novel drugs with hitherto unknown properties.

For simulated data, we notice that the inversion procedure provides quite accurate estimates. Systematically, we observe a tendency to overestimate the block of both the I_{Kr} and I_{CaL} currents. As alluded to above, it is notoriously difficult to distinguish reduced I_{Kr} from increased I_{CaL} and vice versa. This can be improved (FIG. 7 and FIG. 8), by using several values of the extracellular calcium concentration, but the problem is not completely removed.

E. Repolarization Waves in Bidomain Simulations

As mentioned above, the bidomain model has been used by many authors (see e.g., [18, 16, 19, 20, 14]) to simulate the electrophysiology of collections of hiPSC-CMs. One problem pointed out by several authors is the lack of a repolarization wave in the simulated results although the repolarization wave is clearly present in measurements of the extracellular potential; see, e.g., FIG. 3 and FIG. 4 of [18]. This feature of the bidomain solution is repaired by introducing heterogeneities in the tissue which lead to a repolarization wave.

In FIG. 9, we show that there is indeed a repolarization wave present in the extracellular data obtained from collections of hiPSC-CMs. The repolarization wave is also present in our bidomain simulations, but the strength of the wave depends critically on the intracellular conductivity, σᵢ. The repolarization wave becomes stronger as σᵢ is reduced. Since the intracellular conductivity represents the geometrical average of the intercellular conductivity (regulated by gap junctions) and the cytosolic conductivity, it is reasonable to use a reduced value of σᵢ for immature cells, since the gap junctions are most likely less developed in collections of hiPSC-CMs than for collections of adult cardiomyocytes. In [18], it is argued that it is reasonable to introduce heterogeneities in the tissue and we agree. However, our results indicate that it is not necessary in order to see a repolarization wave in the simulation results.

### V. METHOD OF DETERMINING TISSUE PROPERTIES

Embodiments can provide techniques to accurately estimate properties (e.g., ion current densities, such as for sodium channels) of a test tissue including cardiomyocyte (CM) cells, using measurements of the membrane potential V, the cytosolic calcium concentration Ca, and the extracellular potential U. As examples, the test tissue can include an immature cell (e.g., a stem cell) or an animal cell, as they can be easily obtained for performing measurements.

The tissue properties can be determined from parameters of a tissue model that is inverted using these measurements (U, Ca, and U). The tissue property can equal or be derived from one or more parameters of the tissue model. The ion current densities can be specified as a percentage block (e.g., λ as specified above) of a normal current density for a particular ion. Example ion channels include sodium, calcium, and potassium. Multiple channels can exist for each ion, e.g., fast and late channels.

A comparison of the tissue properties for a control CM and a drug-treated CM can provide an effect of a particular dosage of a drug on CM cells and more generally on the heart. Such a comparison can be performed by a comparison to a threshold that corresponds to the control CM. As another example, the comparison can be made directly between the estimated property for the control CM and the estimated property for the drug-treated CM. As yet another example, a mature drug-treated tissue model can be determined using a maturation function Q (e.g., a matrix) that is applied to the drug-treated tissue model. Then, the estimated property determined from the mature drug-treated tissue model can be compared to a property determined from a mature base tissue model or compared to a corresponding threshold.

The model can be determined using an optimization of a cost function to learn parameters of the model, as explained above. The parameters can be defined in various, e.g., using finite element or fine difference. Other basis functions could also be used, e.g., periodic functions (e.g., Fourier series) or localized functions (e.g., wavelets, Gaussians, etc.). Embodiments can define the tissue model using partial differential equations, so as to incorporate the optical measurements and the electrical measurements into a single model.

A. Measurements

In some embodiments, Vₘ and/or Caᵢ are optically measured, e.g., using a voltage- and/or calcium-sensitive indicator or sensor (e.g., a dye), respectively. For optical measurements, the indicator or sensor will typically be fluorescent. Indicator(s) and/or sensor(s) that produce signals in the visible, far-red, and/or near-infrared spectrum can be used. Such measurements can be made using a microphysiological system (MPS). The use of microphysiological systems to measure Vₘ and/or Caᵢ is described in the Examples section below and also described, e.g., in Anurag Mathur. et al. Scientific reports (2015) 5:8883, which is incorporated herein in its entirety for all purposes.

As a brief non-limiting example, an MPS system is loaded and matured prior to drug exposure. On the day upon which studies are performed, freshly measured drug is dissolved into an appropriate solvent (e.g., DMSO) or cell media and serially diluted. Each concentration of the drug to be tested is preheated (e.g., for about 15-20 minutes) in a water bath (e.g., at about 37° C.) and subsequently sequentially injected in the device. At each dose, after an appropriate exposure time (e.g., about 5 minutes), the drug's response on the microtissue is recorded using a suitable imaging device (e.g., a Nikon Eclipse TE300 microscope fitted with a QImaging camera). Fluorescent images are acquired at a suitable frame rate (e.g., at 100 frames per second) using appropriate filters to capture fluorescence from the selected indicator(s) (e.g., GCaMP for Caᵢ and/or and BeRST-1 for Vₘ). Images are obtained across the entire chip for a suitable length of time (e.g., about 6-8 seconds), with cell excitation paced at an appropriate frequency (e.g., about 1 Hz), to capture multiple beats for processing. Fluorescence videos are then analyzed using software (e.g., Python software utilizing the open source Bio-Formats tool or other suitable software) to produce characteristic voltage and calcium waveforms for each chip and tested drug dose. Briefly, for each analysis, the fluorescent signal for the entire visual field is averaged, excluding pixels which do not change significantly in intensity over the acquisition. The signal is then smoothed (e.g., using a 3 point median filter), and an appropriate number (e.g., 5-7) individual action potentials or calcium transients are overlayed by aligning the maximum dF/dt and then averaged into a single transient.

In some embodiments, Vₘ and/or Caᵢ (e.g., in an immature CM) are detected by observing or recording a signal (e.g., optical signal). In particular embodiments, the signal is displayed on a device (e.g., computer monitor or other electronic display) and/or recorded on an analog and/or digital recording medium. As non-limiting examples, devices that can be used to detect a signal (e.g., optical signal, such as a fluorescent signal) include, CCD cameras, video cameras, photographic film, laser-scanning devices, fluorometers, photodiodes, quantum counters, epifluorescence microscopes, scanning microscopes, fluorescence microplate readers, and signal amplification using photomultiplier tubes. Typically, the signal-detecting device will need to be coupled to a microscope. In addition, appropriate filter(s) may be used, depending on the choice of indicator(s) to detect Vₘ- and/or Caᵢ-related signals. In some embodiments, detecting a fluorescent signal comprises detecting a change in fluorescence (e.g., a change in fluorescence intensity, fluorescence excitation or emission wavelength distribution, fluorescent lifetime, and/or fluorescence polarization).

For measurement of Vₘ, in some embodiments, the signal is generated using a fluorescent voltage indicator or sensor (e.g., a voltage-sensitive fluorescent dye). Voltage-sensitive dyes change their absorbance or fluorescence when there is a change in the membrane potential of the cell to which they are bound. Generally, voltage-sensitive dyes are grouped into slow dyes and fast dyes. Slow dyes respond to changes in voltage with a time constant of milliseconds to seconds. Fast dyes respond with time constants in the microsecond range. A non-limiting example of a suitable fluorescent voltage indicator is Berkeley Red Sensor of Transmembrane Potential (BeRST1). Non-limiting examples of other indicators include carbocyanines, rhodamines, ionic oxonols, and those based on aminonaphthylethenylpyridinium (ANEP) and aminonapthylbutydienylquinolinium (ANBDQ) backbones (e.g., Di-1-ANEPEQ, Di-1-ANEPPQ, Di-2-ANEPEQ, Di-2-ANBDQPQ, Di-2-AN(F)EPPTEA, Di-3-ANEPPDHQ, Di-4-ANEPPS, Di-4-AN(F)EPPTEA, Di-4-AN(F)EP(F)PTEA, Di-4-ANEP(F2)PTEA, Di-4-ANBDQBS, Di-4-ANBDQPQ, Di-4-ANEQ(F)PTEA, and Di-8-ANEPPS).

For measurement of Caᵢ, in some embodiments, the signal is generated using a fluorescent calcium indicator or sensor (e.g., a calcium-sensitive fluorescent dye). In some embodiments, the signal is proportional (e.g., directly proportional) to the intracellular calcium concentration. In other embodiments, the signal is inversely proportional to the intracellular calcium concentration. Non-limiting examples of suitable fluorescent calcium indicators or sensors include GCaP, bis-fura-2, BTC, calcium green-1, calcium green-2, calcium green-5n, calcium orange, calcium crimson, fluo-3, fluo-4, fluo-5F, fluo-4FF, fluo-5N, fura-2, fura-4f, fura-6F, fura-FF, fura red, indo-1, mag-fluo-4, mag-fura-2, mag-indo-1, magnesium green, Oregon green 488 BAPTA-1, Oregon green 488 BAPTA-2, Oregon green 488 BAPTA-6F, Oregon green 488 BAPTA-5N, quin-2, rhod-2, rhod-3, rhod-FF, rhod-5N, X-rhod-1, X-rhod-5F, a genetically-encoded sensor, analogs thereof, acetoxymethyl esters thereof, acetate esters thereof, water soluble salts thereof, dextran variants thereof, cadaverine variants thereof, thiol-reactive iodoacetamides thereof, and combinations thereof.

In some embodiments, a non-ratiometric fluorescent calcium indicator or sensor is used, wherein the intracellular calcium concentration is determined by a relative increase in fluorescence intensity. Non-ratiometric indicators or sensors primarily work in the visible wavelength range, and are advantageous in that the use of a single excitation wavelength allows for simpler instrumentation and/or simultaneous observation of other parameters. In some embodiments, the relationship between fluorescence and the absolute calcium concentration (e.g., absolute intracellular calcium concentration) is represented by the following equation:

[Ca²⁺]=K_{d}×(F−Fₘᵢₙ)/(Fₘₐₓ−F)

where K_{d} is the dissociation constant of the fluorescent indicator and Fₘᵢₙ and Fₘₐₓ are fluorescence values obtained at minimum and maximum calcium concentrations (e.g., 0 and saturating values). K_{d} is commonly determined for each experimental condition, as K_{d} can be modified by factors such as pH, protein concentration, ionic strength, and lipid interactions.

In some embodiments, a ratiometric fluorescent calcium indicator or sensor is used, wherein the intracellular calcium concentration is measured as a ratio between two fluorescence intensity values that are measured at two different excitation or emission wavelengths. Ratiometric indicators or sensors are advantageous in that they allow for correction for unequal dye loading, bleaching, or focal plane shift.

The extracellular potential can be measured as described herein, e.g., with respect to FIG. 1. For example, the mean extracellular potential measured at the grid points overlapping the location of electrodes within the tissue. Multielectrode arrays can be used as described herein.

In some embodiments, more than one measurement of U, Vₘ, and/or Caᵢ is obtained in the control test tissue, e.g., of immature CMs. In some embodiments, more than one measurement of U, Vₘ, and/or Caᵢ is obtained in the drug-treated tissue. When U, Vₘ, and Caᵢ are measured, the number of measurements can be different, or they can be the same. The number of measurements will depend on the measurement frequency and the length of time that measurements are made. In some embodiments, about 1, 2, 3, 4, 5, 6, 7, 8, 9, 10, 20, 30, 40, 50, 60, 70, 80, 90, 100, 200, 300, 400, 500, 600, 700, 800, 900, 1,000, 2,000, 3,000, 4,000, 5,000, 6,000, 7,000, 8,000, 9,000, 10,000, 20,000, 30,000, 40,000, 50,000, 60,000, 70,000, 80,000, 90,000, 100,000, 200,000, 300,000, 400,000, 500,000, 600,000, 700,000, 800,000, 900,000, 1,000,000, 1,500,000, 2,000,000, 2,500,000, 3,000,000, 3,500,000, 4,000,000, 4,500,000, 5,000,000, 5,500,000, or more measurements of U, Vₘ, and/or Caᵢ (e.g., in a control and/or drug-treated immature tissue) are obtained.

In some embodiments, at least about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 U, Vₘ, and/or Caᵢ measurements are made per second. In some embodiments, about 100, 150, 200, 250, 300, 350, 400, 450, 500, 550, 600, 650, 700, 750, 800, 850, 900, 950, 1,000, 1,100, 1,150, 1,200, 1,250, 1,300, 1,350, 1,400, 1,450, 1,500, 1,550, 1,600, 1,650, 1,700, 1,750, 1,800, 1,850, 1,900, 1,950, 2,000, 2,100, 2,150, 2,200, 2,250, 2,300, 2,350, 2,400, 2,450, 2,500, 2,550, 2,600, 2,650, 2,700, 2,750, 2,800, 2,850, 2,900, 2,950, 3,000 U, Vₘ and/or Caᵢ measurements are made per second. When U, Vₘ, and Caᵢ are measured, the measurement frequencies for U, Vₘ, and Caᵢ can be different, or they can be the same.

In some embodiments, U, Vₘ, and Caᵢ measurements are made for at least about 1 second. In some embodiments, U, Vₘ, and Caᵢ measurements are made for about least about 2, 3,4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60 seconds. In some embodiments, U, Vₘ, and Caᵢ measurements are made for about least about 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 minutes.

In some embodiments, U, Vₘ, and Caᵢ measurements are made for between about 1 second and about 60 seconds, between about 1 second and about 50 seconds, between about 1 second and about 40 seconds, between about 1 second and about 30 seconds, between about 1 second and about 20 seconds, or between about 1 second and about 10 seconds. In some embodiments, U, Vₘ, and Caᵢ measurements are made for between about 1 minute and about 30 minutes, between about 1 minute and about 25 minutes, between about 1 minute and about 20 minutes, between about 1 minute and about 15 minutes, between about 1 minute and about 10 minutes, between about 1 minute and about 9 minutes, between about 1 minute and about 8 minutes, between about 1 minute and about 7 minutes, between about 1 minute and about 6 minutes, between about 1 minute and about 5 minutes, between about 1 minute and about 4 minutes, between about 1 minute and about 3 minutes, or between about 1 minute and about 2 minutes.

In some embodiments, the control immature tissue and the drug-treated immature CM are the same (i.e., one or more U, Vₘ, and Caᵢ measurements are obtained in the control tissue (e.g., of immature CM), and then one or more U, Vₘ, and Caᵢ measurements are obtained in the same immature CM after the immature CM has been contacted with the composition comprising the sufficient amount of the drug). In other embodiments, the control test tissue and the drug-treated tissue are different cells. In some embodiments, measurements U, Vₘ, and Caᵢ are obtained in one control and/or one drug-treated immature CM. In other embodiments, measurements of U, Vₘ, and Caᵢ are obtained in 2, 3, 4, 5, 6, 7, 8, 9, 10, 11, 12, 13, 14, 15, 16, 17, 18, 19, 20, or more control and/or drug-treated tissue.

In some embodiments, the composition comprising the sufficient amount of the drug is contacted with the tissue for at least about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60 seconds before the one or more second measurements of Vₘ and/or the one or more second measurements of Caᵢ are obtained. In some embodiments, the composition comprising the sufficient amount of the drug is contacted with the tissue for at least about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60 minutes before the one or more second measurements of Vₘ and/or the one or more second measurements of Caᵢ are obtained.

B. Relation Among Models

The parameters of a base model can be used as an initial estimate for the inverting process. Such a base model is not calibrated to the individual data traits. It can be considered to be an unformed model. After taking the measurements, a control model can be determined using the measurements, so the control model is calibrated to the individual. The control model represents control data of a test cell(s) and extracellular properties. After treating the test cell(s) with a drug, measurements of the control cell(s) can provide a drug control model. As examples, the control cell(s) can be immature cells (e.g., stem cells) or cells from a different animal. By comparing the control and the drug model, one can find the mechanism of action.

A Q function (e.g., a diagonal maturation matrix) can provide a mapping from the control model to a generic adult model. The Q can be used to map the drug control model to the drug mature model. The Q can scale the parameters of the model (e.g., the ion currents). Thus, once the parameters of the control model are determined, the system can calculate the scaling for all those parameters that will provide a mature model. Further details on the relation between models can be found in U.S. Patent Publication No. 2019/0242879, which relates to cell models, whereas the models in this disclosure are tissue models, as they include extra cellular properties (e.g., extracellular potential U).

As to the determination of the drug effects, embodiments can compare the mature model to the mature drug model. However, one might just look at the parameters of the mature drug model. For example, someone may just want to know what the sodium channel value is for the drug control model. One could see that the drug effects the sodium channel significantly, thereby providing an indicate that the chemistry of the drug should be changed. For instance, a criteria can be that a sodium block is never greater than a threshold, e.g., as part of a safety protocol. However, if the block for a current is low or moderate level, a mapping to a mature model can provide more detailed information about how significant the block is expected to be in a person.

C. Determining Drug Effects

In order to determine the effects of a drug (e.g., on a mature CM) or to select a drug (e.g., that does not have a proarrhythmic effect on a mature CM) according to methods of the present invention, the mature base CM model and the mature drug-treated CM model (i.e., parameterized by vectors p^{M} and p^{M,D}, respectively) are used to generate a simulated mature control AP and a simulated mature drug-treated AP, respectively. One or more morphological properties are then compared between the simulated mature control AP and the simulated mature drug-treated AP in order to calculate difference(s) between the two action potentials, and the difference(s) are used to determine the effect(s) of the drug and/or to select a drug. In this section, a CM model refers to a tissue model.

In some embodiments, more than one simulated mature control AP and/or more than one simulated mature drug-treated AP are generated. In particular embodiments, a number of simulated APs (e.g., at least 2, 3, 4, 5, 6, 7, 8, 9, 10, 15, 20, 30, 40, 50, 60, 70, 80, 90, 100, 200, 300, 400, 500, 600, 700, 800, 900, 1,000, or more) are generated before specific simulated APs are selected comparison of morphological properties. In some instances, the number of simulated APs that are generated depends on the simulated pacing rate, choice of specific CM model, mathematical method used to solve equations in the CM model, how quickly parameter(s) in the CM model approach steady state, etc. In some embodiments, only one simulated mature control AP and/or only one simulated mature drug-treated AP are selected for comparison of morphological properties. In some embodiments, more than one (e.g., at least 2, 3, 4, 5, 6, 7, 8, 9, 10, 11, 12, 13, 14, 15, 16, 17, 18, 19, 20, or more) simulated mature control APs and/or simulated mature drug-treated APs are selected for comparison of morphological properties.

In some embodiments, the one or more morphological properties (e.g., that are compared between simulated mature control and drug-treated APs) are selected from the group consisting of APD_{V,n}, APD_{Ca,n}, maximum Vₘ upstroke velocity ((dv/dt)ₘₐₓ), resting membrane potential, maximum Caᵢ upstroke velocity ((dc/dt)ₘₐₓ), Caᵢ (e.g., peak Caᵢ), and a combination thereof. It is understood that each instance of n is independently selected. Furthermore, multiple instances of n can be selected (e.g., APD_{V,30}, APD_{V,50}, and/or APD_{V,80} can be used in one comparison, and/or APD_{Ca,30}, APD_{Ca,50}, and/or APD_{Ca,80}, can be used in one comparison). In some embodiments, more than 1 (e.g., 2, 3, 4, 5, 6 or more) morphological property is compared. In some embodiments, the effect of more than 1 (e.g., 2, 3, 4, 5, 6, or more) drug on a mature CM is determined, or more than 1 (e.g., 2, 3, 4, 5, 6, or more) drug is selected that does not have a proarrhythmic effect of a mature CM.

In some embodiments, a drug is determined to have a proarrhythmic effect (e.g., on a mature CM) when at least one of the morphological properties (e.g., APD_{V,n} or APD_{Ca,n}) in the simulated mature drug-treated AP is higher (e.g., at least about 0.1-fold, 0.2-fold, 0.3-fold, 0.4-fold, 0.5-fold, 0.6-fold 0.7-fold, 0.8-fold, 0.9-fold, 1-fold 1.1-fold, 1.2-fold, 1.3-fold, 1.4-fold, 1.5-fold, 1.6-fold, 1.7-fold, 1.8-fold, 1.9-fold, 2-fold, 2.5-fold, 3-fold, 3.5-fold, 4-fold, 4.5-fold, 5-fold, 5.5-fold, 6-fold, 6.5-fold, 7-fold, 7.5-fold, 8-fold, 8.5-fold, 9-fold, 9.5-fold, or 10-fold higher) compared to the corresponding morphological property in the simulated mature control AP.

In some embodiments, a drug is determined to have a proarrhythmic effect (e.g., on a mature CM) when at least one of the morphological properties (e.g., (dv/dt)ₘₐₓ) in the simulated mature drug-treated AP is lower (e.g., at least about 0.1-fold, 0.2-fold, 0.3-fold, 0.4-fold, 0.5-fold, 0.6-fold, 0.7-fold, 0.8-fold, 0.9-fold, 1-fold 1.1-fold, 1.2-fold, 1.3-fold, 1.4-fold, 1.5-fold, 1.6-fold, 1.7-fold, 1.8-fold, 1.9-fold, 2-fold, 2.5-fold, 3-fold, 3.5-fold, 4-fold, 4.5-fold, 5-fold, 5.5-fold, 6-fold, 6.5-fold, 7-fold, 7.5-fold, 8-fold, 8.5-fold, 9-fold, 9.5-fold, or 10-fold lower) compared to the corresponding morphological property in the simulated mature control AP.

In some embodiments, a subject (e.g., a patient) is not administered the drug when the drug is determined to have a proarrhythmic effect (e.g., on a mature CM).

In some embodiments, a drug is selected (e.g., for a subject such as a patient) or is determined to not have a proarrhythmic effect (e.g., on a mature CM) when the morphological property (e.g., APD_{V,n} or APD_{Ca,n}) in the simulated mature control AP is less than about 0.1-fold, 0.2-fold, 0.3-fold, 0.4-fold, 0.5-fold, 0.6-fold, 0.7-fold, 0.8-fold, 0.9-fold, 1-fold 1.1-fold, 1.2-fold, 1.3-fold, 1.4-fold, 1.5-fold, 1.6-fold, 1.7-fold, 1.8-fold, 1.9-fold, 2-fold, 2.5-fold, 3-fold, 3.5-fold, 4-fold, 4.5-fold, 5-fold, 5.5-fold, 6-fold, 6.5-fold, 7-fold, 7.5-fold, 8-fold, 8.5-fold, 9-fold, 9.5-fold, or 10-fold lower compared to the morphological property in the simulated mature drug-treated AP.

In some embodiments, a drug is selected (e.g., for a subject such as a patient) or is determined to not have a proarrhythmic effect (e.g., on a mature CM) when the morphological property (e.g., (dv/dt)ₘₐₓ) in the simulated mature control AP is less than about 0.1-fold, 0.2-fold, 0.3-fold, 0.4-fold, 0.5-fold, 0.6-fold, 0.7-fold, 0.8-fold, 0.9-fold, 1-fold 1.1-fold, 1.2-fold, 1.3-fold, 1.4-fold, 1.5-fold, 1.6-fold, 1.7-fold, 1.8-fold, 1.9-fold, 2-fold, 2.5-fold, 3-fold, 3.5-fold, 4-fold, 4.5-fold, 5-fold, 5.5-fold, 6-fold, 6.5-fold, 7-fold, 7.5-fold, 8-fold, 8.5-fold, 9-fold, 9.5-fold, or 10-fold higher compared to the morphological property in the simulated mature drug-treated AP.

In some embodiments, the methods of the present invention further comprise administration of a drug (e.g., a drug selected according to methods of the present invention) to a subject (e.g., a patient or a subject in need thereof).

D. Flowchart

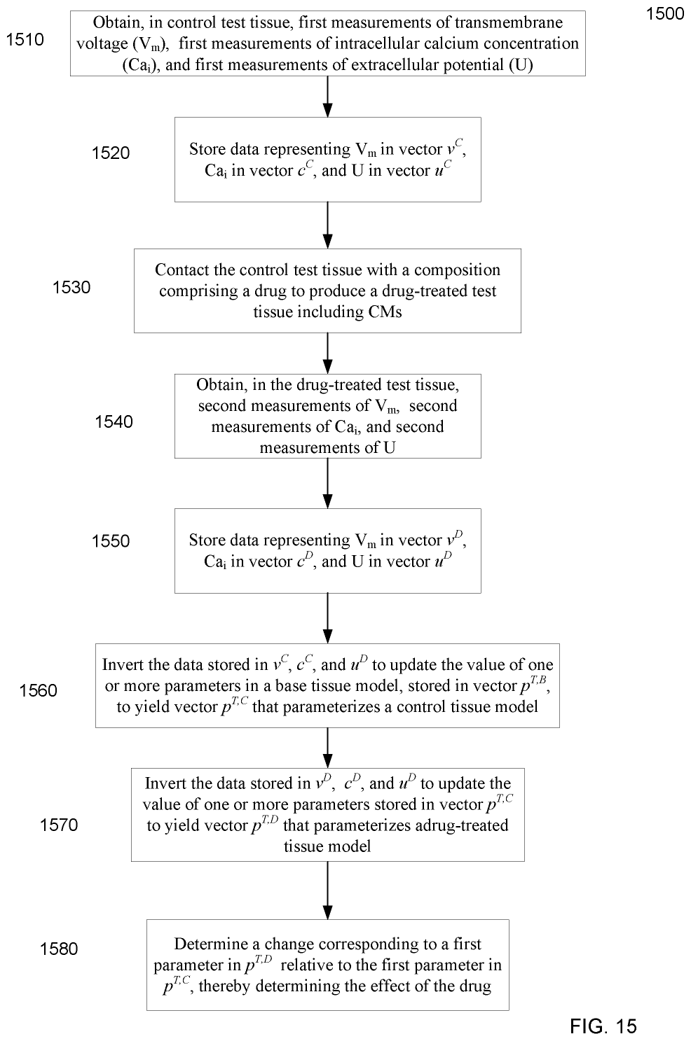

FIG. 15 is flowchart illustrating a method 1500 for determining an effect of a drug on a cardiomyocyte (CM) according to embodiments of the present disclosure. Method 1500 can be used to determine the effect of a drug on cells. An example of the cells includes cardiomyocytes (CMs), e.g., mature CMs.

At block 1510, first measurements of transmembrane voltage (Vₘ), first measurements of intracellular calcium concentration (Caᵢ), and first measurements of extracellular potential (U) are obtained over time in control test tissue. The control test tissue can includes cardiomyocyte (CMs). The control test tissue can include immature CMs, e.g., stem cells. The measurements of U can be electrical measurements, which can be performed using a grid of electrodes as described herein. The measurements of Vₘ and Caᵢ can be optical measurements, e.g., as described herein. The measurements can correspond to a time series of measurement of a portion or all of a cycle of a heartbeat.

At block 1520, data representing the first measurements of Vₘ are stored in a vector v^{C}, data representing said first measurements of Caᵢ are stored in a vector c^{C}, and data representing said first measurements of U in a vector u^{C}. The vectors can store the data as an array of values. The order of the values can be known, e.g., as defined in a template. The data stored in the vectors can be the raw data from the first measurements or results of an analysis of the raw data, e.g., a global average or statistical values relating to increases or decreases relative to a peak. Such data can include values described for FIG. 4. Such data can represent a shape of the first measurements over time, e.g., during a heartbeat, such an upstroke of a heartbeat.

The vector p does not necessarily need to contain all of the parameters that are present in a given computational tissue model, so long as p contains the parameters that are relevant to fitting the computational models to the data obtained from the control and drug-treated tissue. The parameters contained in vector p may represent any number of components, properties, or properties of components that are present within a simulated tissue. The one or more parameters may represent, as non-limiting examples, the number of one or more proteins in the simulated tissue, the dynamics of one or more proteins in the simulated tissue, the volume of the simulated tissue, the surface area of the simulated tissue, the cell membrane capacitance of the simulated tissue, or a combination thereof. The one or more proteins may be, as non-limiting examples, transmembrane protein(s) (e.g., ion channel(s), ion exchanger(s), ion pump(s), or any combination thereof), sarcoplasmic reticulum (SR) protein(s) (e.g., SR release and/or uptake protein(s)), or any combination thereof. The one or more proteins (e.g., ion channel(s), exchanger(s), pump(s), release protein(s), and/or uptake protein(s)) may transport or permit the passage of ions (sodium, potassium, calcium, magnesium, chloride, bicarbonate, protons, hydroxide ions, or other relevant ions), proteins, or other species, e.g., into or out of the simulated cells, or into or out of a particular compartment, subspace, or organelle within the simulated cells. In some embodiments, the one or more parameters represent a maximum conductance (e.g., of an ion channel) and/or a maximum rate (e.g., of an ion exchanger or pump). In some embodiments, the one or more parameters represent a value (e.g., a time constant) that is associated with the rate of opening or closing of an ion channel (e.g., an ion channel gating variable), an association constant, a dissociation constant, or a combination thereof.

At block 1530, the control test tissue is contacted with a composition comprising a drug to produce a drug-treated test tissue including CMs. The amount of drug can correspond to a different doses that might be expected to be used for a subject. The drug can be designed to treat any number of diseases, i.e., not necessarily heart related.

In some embodiments, the composition is contacted with the control test tissue for at least about 15 minutes before the one or more second measurements are obtained. In some embodiments, the drug is a calcium channel blocker, a potassium channel blocker, a sodium channel blocker, or a combination thereof. In some embodiments, the effects of two or more drugs are determined.

At block 1540, second measurements of Vₘ, second measurements of Caᵢ, and second measurements of U) are obtained over time in the drug-treated test tissue. The second measurements may be performed in a same manner as the first measurements. In some embodiments, at least about 100 measurements are made per second. In particular embodiments, between about 100 and about 3,000 measurements are made per second. In some embodiments, the measurements are made for at least about 1 second. In particular embodiments, the measurements are made for between about 1 second and about 30 minutes

In some embodiments, the control test tissue and the drug-treated test tissue are different cells, but of the same type, and thus both measurements can be determined using control test tissue. For example, the first measurements could be on one set of CM cells, and the second measurements could be performed on a different set of CM cells. In other embodiments, the cells are the same.

At block 1550, data representing said second measurements of Vₘ are stored in vector v^{D}, data representing said second measurements of Caᵢ are stored in vector c^{D}, and data representing said second measurements of U are stored in vector u^{D}. The data can be of a same type as measured in block 1520.

At block 1560, the data stored in v^{C}, c^{C}, and u^{C} is inverted to update a value of one or more parameters in a base tissue model (which is stored in vector p^{T,B}) to yield vector p^{T,C} that parameterizes a control tissue model. The base tissue model can be an established model (e.g., of an immature tissue model) that is known. In one implementation, each of the control tissue model and the base tissue model is a bidomain model. The base tissue model is a ‘generalized’ model that should be close to the control model, and can used as a starting point to determine the control model.

The inversion may be performed as described in section II. For example, the inversion can solve for the parameters of the model that provide a best approximation for predicting the measure values in v^{C}, c^{C}, and u^{C}. For a set of parameters, the model can predict a set of expected measured values, where data (e.g., statistical values) of such expected measured values can be compared to the actual measured values to determine a cost value, as determined by a cost function, e.g., as defined in section II.B.1. Accordingly, the cost function can include a term for the extracellular potential and a term for a conduction velocity. Other example terms include action potential and calcium transient durations, membrane potential integral, calcium transient time to peak, and membrane rise time.

Accordingly, in some embodiments, inverting the data can comprise minimizing a cost function. The cost function can measure a difference in shapes over time of the measured U, V, and Ca values relative to predicted values using a respective tissue model. The data in the vectors v^{C}, c^{C}, and u^{C} can define the shapes of the respective properties of the system. The predicted values can be determined by solving a partial differential equation of the respective tissue model. The partial differential equation can include gradients of Vₘ and U. The predicted Ca values can be determined via an ordination differential equation.

The choice of the parameters for the base model can be determined by, e.g., the particular organism of interest (e.g., human, dog, pig, guinea pig, mouse, or rat), cell type of interest (e.g., atrial or ventricular cell), the particular drug(s) being studied, particular electrophysiological properties or effects of interest (e.g., that are potentially modulated by the drug), specific diseases, pathologies, or mutations, etc.

At block 1570, the data stored in v^{D}, c^{D}, and u^{D} are inverted to update the value of one or more parameters stored in a vector p^{T,C} to yield a vector p^{T,D} that parameterizes a drug-treated tissue model. The inversion may be performed in a similar manner as in block 1560, but using the data for the second measurements. The drug-treated tissue model and the control model can have a same functional structure, but with the drug-treated tissue model being trained using the drug measurements. The use of the extracellular potential can provide an increased accuracy in p^{T,C} and p^{T,D}, yielding better accuracy in estimates of the parameters, e.g., a sodium current. The inversion in blocks 1560 and 1570 can be performed simultaneously, as described in section VII.

At block 1580, a change corresponding to a first parameter in p^{T,D} relative to the first parameter in p^{T,C} is determined. The change can provide the effect of the drug. The change can be used to determine whether the drug is safe. If the change is too high, then the drug can be deemed not safe.

Embodiments can further include updating one or more values in a diagonal maturation matrix Q such that an equality Qp^{T,C}=p^{M} is satisfied, wherein p^{M} is a vector that parameterizes a mature base tissue model. Before Q is updated, Q satisfies an equality p^{M}=Qp^{T,B}. Thus, as described above, Q provides a transformation of parameters from the base tissue model to a mature base tissue model. This same Q can also provide a transformation from the drug-treated tissue model to a mature drug-treated CM model. To do this, Q can multiply p^{T,D} to generate vector p^{M,D}, where p^{M,D} parameterizes the mature drug-treated CM model. Accordingly, a model can be mapped from the IM case to the M case by multiplying a parameter vector by a diagonal maturation matrix. Multiplying a vector p by Q may change any number of model features in a computational CM model that is parameterized by p. As non-limiting examples, multiplying a vector p by Q may change the number of one or more proteins, the cell volume, and/or the cell surface area in the CM model that is parameterized by p. The one or more proteins may include, as non-limiting examples, membrane proteins (e.g., ion channels, pumps, and/or exchangers), sarcoplasmic reticulum (SR) release proteins, SR uptake proteins, or combinations thereof.

The mature drug-treated CM model can be used to determine the effect of the drug. For example, at least one of the parameters stored in p^{M,D} can be compared to a threshold to determine whether the effect of the drug is unacceptable. The acceptability of the drug is defined by the threshold, which can be determined based knowledge when a sodium level causes damage to a cell. As another example, a difference can be determined between a parameter stored in p^{M,D} and a corresponding parameter in p^{M}. The difference can be compared to a threshold to determine whether the effect of the drug is unacceptable. An example parameter for such comparisons is a sodium current. Accordingly, the base tissue model can correspond to immature tissue including immature CMs, and the effect of the drug can be determined for mature CMs by scaling the change, e.g., using Q.

As a further example of determining the effect of the drug, the base tissue model and the drug-treated tissue model can be used to generate a simulated mature control action potential (AP) and a simulated mature drug-treated AP. The AP can be determined using the parameter p^{M,D} defined above, by p^{M,D}=Qp^{T,D}. One or more morphological properties of the simulated mature control AP and the simulated mature drug-treated AP can be compared to calculate a difference, thereby determining the effect of the drug. In some implementations, the drug can be determined not have a proarrhythmic effect based on the difference, and the drug can be selected when it does not have a proarrhythmic effect.

In some embodiments, the one or more morphological properties are selected from the group consisting of APD_{V,n}, APD_{Ca,n}, maximum Vₘ upstroke velocity ((dv/dt)ₘₐₓ), resting membrane potential, maximum Caᵢ upstroke velocity ((dc/dt)ₘₐₓ), peak Caᵢ, and a combination thereof; wherein APD_{V,n} and APD_{Ca,n}, are the duration of the Vₘ action potential and calcium transients, respectively; and wherein each value of n is independently selected and represents a percentage of the maximum Vₘ value. In some instances, each value of n is independently selected from the group consisting of 30, 50, and 80.

In some embodiments, the drug is determined to have a proarrhythmic effect when at least one of the morphological properties in the simulated mature drug-treated AP is at least about 1.5-fold higher or lower compared to the corresponding morphological property in the simulated mature control AP. In particular embodiments, the drug is determined to have a proarrhythmic effect when at least one of the morphological properties in the simulated mature drug-treated AP is at least about 3-fold higher or lower.

A parameter of the drug-treated tissue model can be used directly to determine the effect of the drug. For example, at least one of the parameters stored in p^{T,D} can be compared to a threshold to determine whether the effect of the drug is unacceptable. The threshold may be determined relative to a corresponding parameter of the control tissue model as stored in p^{T,C}. As another example, a difference between a parameter stored in p^{T,D} and a corresponding parameter in p^{T,C} can be determined, and compared to a threshold to determine whether the effect of the drug is unacceptable.

When a drug is determined to be safe (e.g., the effect of the drug is acceptable), the drug can be administered to patients as a treatment, e.g., as part of a trial.

In the results above, we have shown that by using data traces of the membrane potential, the intracellular calcium concentration and the extracellular potential, we can estimate the major sodium, calcium and potassium currents in the base model.

### VI. USE ANIMAL CELLS

In some implementations, the control test tissue can include animal cells of a first animal, e.g., animal CMs. The base tissue model can correspond to a second animal that is different than the first animal. In such situations, a transformation (e.g., as defined by Q) between the animal cell and the mature human cell. When using animal cells, measurements of any one or more of Vₘ, Caᵢ, and/or U may be used.

Animal cells are routinely used as human proxies for assessing mechanisms of action and cardiotoxicity of novel drug candidates, see e.g. [L Carlsson, Journal of Internal Medicine, 259(1):70-80, 2006; S Nattel, G Duker, and L Carlsson, Progress in Biophysics and Molecular Biology, 98(2-3):328-339, 2008]. Often, however, the results, although qualitatively instructive, offer only limited quantitative insights into how these compounds can affect the fine-tuned electrophysiological mechanisms that underlie the cardiac action potential (AP) of humans. This limitation arises from differences in the composition of the cell membrane between animal and human ventricular myocytes, see e.g. [H R Lu, R Marien, A Saels, and F De Clerck, Journal of cardiovascular electrophysiology, 12(1):93-102, 2001; G Gintant, P T Sager, and N Stockbridge, Nature Reviews Drug Discovery, 15(7):457, 2016; N Shanks, R Greek, and J Greek, Philosophy, Ethics, and Humanities in Medicine, 4(1):2, 2009; SR Houser et al., Circulation Research, 111(1):131-150, 2012]. Even when the individual membrane proteins (ion channels) are similar among species, the overall fabrication of the cell membrane differs significantly due to large differences in the densities of the individual ion channels, leading to distinct action potentials, see [G Gintant, P T Sager, and N Stockbridge, Nature Reviews Drug Discovery, 15(7):457, 2016; AG Edwards and W E Louch, Clinical Medicine Insights: Cardiology, 11:1179546816686061, 2017; Z Lu et al., Circulation, 104(8):951-956, 2001].

Here, we show how the similarities of single ion channels between animals and humans can be utilized to devise a method for robust, quantitative translation from animal measurements to human results. Our results demonstrate that measurements of standard electrophysiological biomarkers, such as action potential duration and maximum upstroke velocity, reveal how individual ion channels of animal cardiomyocytes are changed by a drug. These findings, in turn, can be accurately translated to their human counterparts. Accordingly, animal data can be translated to human data using mathematical models of the respective species. Our approach can lead to long-sought quantitative understanding of cardiotoxicity in humans, as based entirely on animal tests.

A. Introduction

We develop model-based methods evaluating whether measurements of drug effects on animal cardiomyocytes can be translated to corresponding drug effects on human cardiomyocytes. The analysis reveals how effects on membrane currents carried by the same ion channel protein in animal and humans can be translated in order to evaluate and understand human ventricular action potentials. This approach provides a framework revealing unwanted side effects early in the development of novel drug candidates. The usefulness of animal models in understanding human electrophysiology is unquestioned, but limited by the inherent difference between the human and animal action potentials; see e.g. [Y Yoshida and S Yamanaka, Circulation Research, 120(12):1958-1968, 2017; L Ye et al., Journal of Cardiovascular Translational Research, 11(5):366-374, 2018; A G Edwards and W E Louch, Clinical Medicine Insights: Cardiology, 11:1179546816686061, 2017]. Often, this gives rise to difficulties in direct translation; even if some of the ion channels involved are functionally nearly identical, the overall composition of membrane ion channels can differ substantially between species. Therefore, block of the same ion channel in two different species may yield very different perturbations of the respective action potentials.

This limitation has serious implications for the overall efficiency and effectiveness of drug development. Currently, this process takes 10 to 15 years, with an average cost of ˜2.5 billion USD [J A DiMasi, H G Grabowski, and R W Hansen, Journal of Health Economics, 47:20-33, 2016]. Preclinical development, including animal testing for safety predictions, accounts for ˜60% of these costs [S M Paul et al., Nature Reviews Drug Discovery, 9(3):203-214, 2010]. However, despite this cost and effort, many promising candidate drugs exhibit toxicity that has not been predicted prior to clinical trials and, ultimately, emerging therapies [Bockorny et al., Acta Haematologica, 128(4):244-247, 2012]; clearly, more quantitatively predictive tools are needed. Given that major currents, underlying the ventricular action potential, are carried by the same or similar ion channels which are well understood (([A G Edwards and W E Louch, Clinical Medicine Insights: Cardiology, 11:1179546816686061, 2017; N Jost et al., The Journal of Physiology, 591(17):4189-4206, 2013; S Zicha et al., American Journal of Physiology-Heart and Circulatory Physiology, 285(4):H1641-H1649, 2003; Z Wang et al., Circulation Research, 84(5):551-561, 1999; P Gemmell et al., PLoS One, 9(2):e90112, 2014; E Grandi, F S Pasqualini, and D M Bers, Journal of Molecular and Cellular Cardiology, 48(1):112-121, 2010]), our approach can be used to translate measured pharmacological effects on the action potential in animal models to those in healthy human adult cardiomyocytes. While meaningful functional differences have been established, the relative conservation of expressed ion channel proteins among mammalian cardiomyocytes provides the basis for application of our methodology for translation of drug effects across species.

B. Results

We demonstrate that it is possible to estimate the effect of a drug candidate on humans solely based on measurements of analogous effects on animal cardiomyocytes. The dog data presented in [N Jost et al., The Journal of Physiology, 591(17):4189-4206, 2013] is employed together with our method to estimate the effect that dofetilide will have on humans. Also, in [N Jost et al., The Journal of Physiology, 591(17):4189-4206, 2013], the effect of dofetilide on human cardiomyocytes was reported, allowing us to assess the quality of our estimates. Additionally, we used the data provided in [P Nemtsas et al., Journal of Molecular and Cellular Cardiology, 48(1):161-171, 2010] on zebrafish to estimate the effect of the drug E-4031 on humans. While the effect on human cardiomycytes is not reported in [P Nemtsas et al., Journal of Molecular and Cellular Cardiology, 48(1):161-171, 2010], this has been reported in [A Bussek et al., Cellular Physiology and Biochemistry, 24(5-6):527-536, 2009; N Jost et al., Circulation, 112(10):1392-1399, 2005], which we again used to assess the accuracy of our estimates. Finally, we used rabbit data presented in [Baczkó et al., Progress in Biophysics and Molecular Biology, 121(2):157-168, 2016] to estimate the effect of sotalol on human myocytes; data on human cardiomyocytes in response to sotalol has been presented in [Baczkó et al., Progress in Biophysics and Molecular Biology, 121(2):157-168, 2016; P Orvos et al., Toxicological Sciences, 168(2):365-380, 2019] and is used here for comparison and assessment.

1. Quantitative Translation from Dog to Human

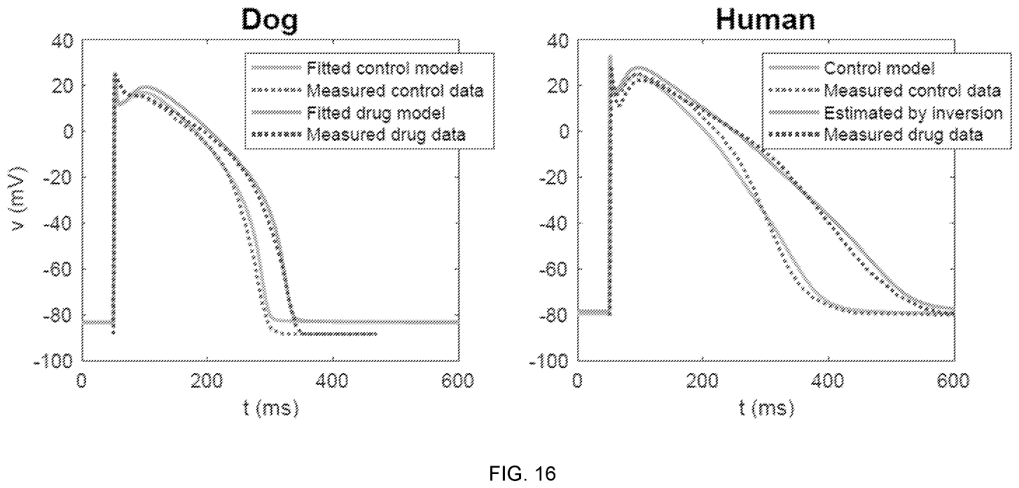

FIG. 16 shows dog (left) and human (right) ventricular action potentials in the control case and in the presence of 50 nM of the I_{Kr} blocker dofetilide. The dotted lines show measured data from [N Jost et al., The Journal of Physiology, 591(17):4189-4206, 2013], and solid lines show simulation results. Note that the drug effect used in the simulation of the human AP was estimated from the dog data.

In FIG. 16, a technique (See section D for more detail) is applied to data published in [N Jost et al., The Journal of Physiology, 591(17):4189-4206, 2013]. In the left panel, experimental data (dotted) is shown for control (blue) and when 50 nM of the I_{Kr} blocker dofetilide is applied (red) to canine myocytes. Solid lines show the results of the mathematical model (See section D). The parameters defining a control model and an IC₅₀ value representing the effect of the drug are estimated and used to model the no drug case (solid blue line) and when the drug has been applied (solid red line).

In the right panel, the control data (dotted blue) and the control mathematical model (solid blue) are illustrated for the human ventricular myocyte. We used the method to estimate the effect of the I_{Kr} blocker on the ventricular AP model of the dog, and this effect is translated (Section D) to the human AP model; this estimated AP (solid red) fits the measured data from [N Jost et al., The Journal of Physiology, 591(17):4189-4206, 2013] very well.

TABLE 4

| | Dog, | Dog, | Human, | Human, | Human, | Human, drug |
|---|---|---|---|---|---|---|
| | control | drug | control | drug | control | estimated based |
| | measured | measured | measured | measured | model | on dog data |
| APD50 (ms) | 201 | +17.5% | 230 | +33.3% | 215 | +36.6% |
| APD90 (ms) | 239 | +18.3% | 312 | +44.3% | 323 | +40.6% |

APD values computed from measured and simulated action potentials in the control case and in the presence of 50 nM of the I_{Kr} blocker dofetilide. The cells in both the experiments [N Jost et al., The Journal of Physiology, 591(17): 4189- 4206, 2013] and in the mathematical model were paced at 1 Hz.

In Table 4, we compare selected biomarkers (APD50 and APD90) for the control case of dog cardiomyocytes (measured), dog cardiomyocytes subjected to the drug (measured), the human control (measured), the human control (model), human cardiomyocytes subjected to the drug (measured) and human cardiomyocytes subjected to the drug (estimated as based on the dog data, as described in section D). All experimental data are taken from [N Jost et al., The Journal of Physiology, 591(17):4189-4206, 2013]. For the dog data, the APD50 is increased by 17.5% when the drug is added while, for human cells, the measured increase in APD50 is 33.3%. When we compare the output from the mathematical model for the control case, and the one where the effect of the drug is added to the model, we observe an increase of 36.6%. Thus, based only on data from the dog, our estimate of the increase of the APD50 for humans differs little from the measured value (36.6 vs 33.3%). The analysis of the APD90 also reveal a similarly small difference (40.6 vs. 44.3%). We conclude that the dog data on APD50/APD90 can be used to estimate the effect of the I_{Kr} blocker dofetilide on human cardiomyocytes well.

2. Quantitative Translation from Zebrafish to Human

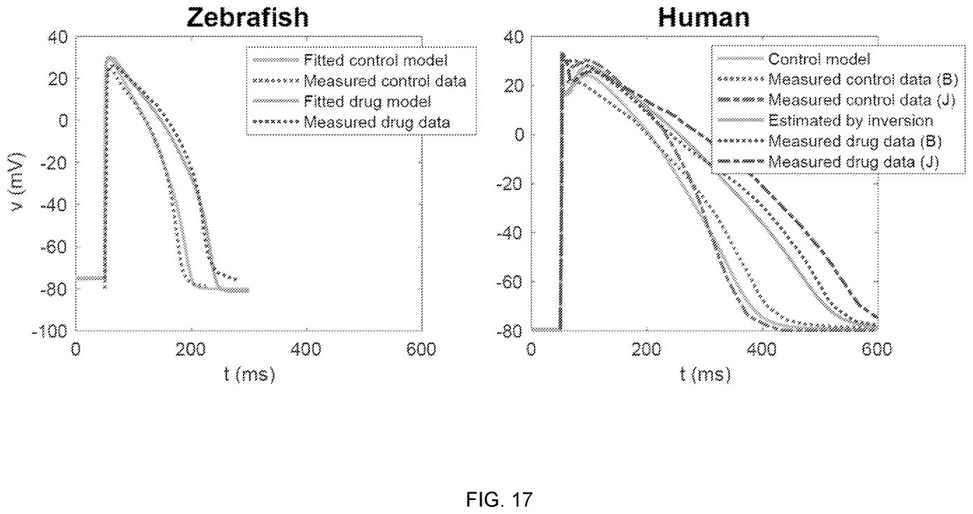

FIG. 17 shows zerbrafish (left) and human (right) ventricular action potentials in the control case and in the presence of 50 nM of the I_{Kr} blocker dofetilide.

In Table 5, the same method was applied to data from zebrafish cardiomyocytes, [P Nemtsas et al., Journal of Molecular and Cellular Cardiology, 48(1):161-171, 2010]. Again, we have focused on experimental APD50 and APD90 values for zebrafish in the control case and the increase caused by the application of 1 μM of the I_{Kr} blocker E-4031. In addition, we report the increases in APD50/APD90 from measurements of human cardiomyocytes exposed to 1 μM E-4031 from Bussek et al. [Jost et al., Circulation, 112(10):1392-1399, 2005] (B) and Jost et al. [N Jost et al., Circulation, 112(10):1392-1399, 2005] (J).

Finally, we report the increases in APD50 and APD90 estimated for human cardiomyocytes as based on the measurements of zebrafish cardiomyocytes. Again, we observe that the APD changes estimated using the mathematical model for the human case agree well with the measured effects for human cells. For APD50, our method estimates a 26.1% increase, while the increase is 27.7% (B) or 54.2% (J) in the experimental data. Similarly, the method estimates a 28.4% increase of APD90, while the experimental data reports a 31.2% (B) or 65.1% (J) increase.

TABLE 5

| | | | | | | | Human drug, |
|---|---|---|---|---|---|---|---|
| | Zebrafish | Zebrafish | Human | Human | | Human | estimated |
| | control | drug | control | drug | | control | based on |
| | measured | measured | measured | measured | | model | zebrafish data |
| APD50 (ms) | 108 | +38.7% | 255 (B) | +27.7% | (B) | 215 | +26.1% |
| | | | | +54.2% | (J) | | |
| APD90 (ms) | 144 | +24.1% | 356 (B) | +31.2% | (B) | 323 | +28.4% |
| | | | | +65.1% | (J) | | |

APD values computed from measured and simulated action potentials in the control case and in the presence of 1 μM of the I_{Kr} blocker E-4031. The zebrafish data is from [P Nemtsas et al., Journal of Molecular and Cellular Cardiology, 48(1): 161-171, 2010] and the human data is from Bussek et al. [A Bussek et al., Cellular Physiology and Biochemistry, 24(5-6): 527-536, 2009] (B) and Jost et al. [N Jost et al., Circulation, 112(10): 1392-1399, 2005] (J).

3. Quantitative translation from rabbit to human

Table 6 shows the result when the same method is applied to rabbit ventricular myocyte data from [I Baczkó et al., Progress in Biophysics and Molecular Biology, 121(2):157-168, 2016]. In this case, two doses (10 μM and 52 μM) of the I_{Kr} blocker sotalol were employed. In addition, [I Baczkó et al., Progress in Biophysics and Molecular Biology, 121(2):157-168, 2016; P Orvos et al., Toxicological Sciences, 168(2):365-380, 2019] provide measured data for human cardiomyocytes exposed to 30 μM sotalol. The column at the extreme right in Table 6 reports the increases in human APD50 and APD90 estimated based on the rabbit data. Both the estimated increase in APD50 (24.9%) and APD90 (27.0%) seem to be in good agreement with the measured human values from [I Baczkó et al., Progress in Biophysics and Molecular Biology, 121(2):157-168, 2016; P Orvos et al., Toxicological Sciences, 168(2):365-380, 2019] (32.3% and 20.6% for APD50 and 37.5% and 28.2% for APD90).

TABLE 6

| | Rabbit, | Rabbit, | Human, | Human, | Human, | Human, drug |
|---|---|---|---|---|---|---|
| | control | drug | control | drug | control | estimated based |
| | measured | measured | measured | measured | model | on rabbit data |
| *APD50 (ms) | 2*162 | +21.2% | 185 (B) | +32.3% | 2*215 | 2* + 24.9% |
| | | (10 μM) | | (30 μM) | | (30 μM) |
| | | | | (B) | | |
| | | +54.3% | 233 (O) | +20.6% | | |
| | | (52 μM) | | (30 μM) | | |
| | | | | (O) | | |
| *APD90 (ms) | 2*194 | +19.6% | 235 (B) | +37.5% | 2*324 | 2* + 27.0% |
| | | (10 μM) | | (30 μM) | | (30 μM) |
| | | | | (B) | | |
| | | +54.1% | 302 (O) | +28.2% | | |
| | | (52 μM) | | (30 μM) | | |
| | | | | (O) | | |

Selected APD values from experimental and simulated action potentials in the control case and in the presence of the I_{Kr} blocker sotalol. The rabbit experimental data is taken from [I Baczkó et al., Progress in Biophysics and Molecular Biology, 121(2): 157-168, 2016], and includes the two drug doses 10 μM and 52 μM. The human experimental data is taken from Baczkó et al. [I Baczkó et al., Progress in Biophysics and Molecular Biology, 121(2): 157-168, 2016] (B) and Orvos et al. [P Orvos et al., Toxicological Sciences, 168(2): 365-380, 2019] (O) and is measured for the drug dose of 30 μM.

C. Discussion

The use of animal models to aid in understanding of human anatomy and physiology is centuries old, see e.g. [A J Hill and PA Iaizzo, In Handbook of cardiac anatomy, physiology, and devices, pages 89-114. Springer, 2015; N Shanks, R Greek, and J Greek, Philosophy, Ethics, and Humanities in Medicine, 4(1):2, 2009].

In the case of the electrophysiology of the vertebrate heart, this type of comparative study [SR Houser et al., Circulation Research, 111(1):131-150, 2012] has led to deep insight into key physiological mechanisms, but it has been difficult to use these models quantitatively due to inherent species differences. More specifically, while animal cells are routinely used as part of drug development, their quantitative usefulness is hampered by the significant differences in human and animal action potential waveforms. At present, the most useful results are obtained by utilizing observed correlations between human and animal biomarkers (see e.g. [J Q X Gong and E A Sobie, NPJ Systems Biology and Applications, 4(1):11, 2018; J Bailey, M Thew, and M Balls, Alternatives to Laboratory Animals, 41(5):335-350, 2013; K Ando et al., Journal of pharmacological sciences, 99(5):487-500, 2005; L Bergenholm et al., Journal of Pharmacological and Toxicological Methods, 79:34-44, 2016; L Bergenholm et al., British Journal of Pharmacology, 174(19):3268-3283, 2017]). However, no mechanistic relationship has, to date, been derived to examine how drug candidates may affect human and animal cardiomyocytes differentially. This limits the predictive insights into the therapeutic or toxic effects on humans. In this disclosure, we have shown how an antiarrhythmic drug effect on human ventricular myocytes can be revealed solely by measuring an analogous effect of the drug on animal ventricular myocytes. This new investigative capability opens a wide variety of possibilities to improve the testing of novel compounds.

Initially, we have shown that canine data can be used to reliably predict the effect of the drug dofetilide on human myocytes as judged by changes in the common biomarkers APD50 and APD90. Similarly, data from zebrafish action potentials was used to estimate the effect of E-4031 on human ventricular myocytes. Finally, the effect of sotalol on rabbit cardiomyocytes was used to estimate the effect of the same drug on human ventricular myocytes, and the results were revealing.

Our method can only be used to estimate a selected drug effect on ion currents that have significant impact on both the animal and the human action potentials. If one current is very small in an animal but very large in a human, drug effects on that current cannot be estimated using the present technique. One such case is illustrated in FIG. 1 of [AG Edwards and WE Louch, Clinical Medicine Insights: Cardiology, 11:1179546816686061, 2017], where the action potential of human, canine, rabbit, guinea pig and mouse are illustrated together with associated currents. The substantial differences between the human and mouse action potentials and underlying ion currents suggest that it is difficult to use our method to predict the effect of drugs on human cardiomyocytes by measuring the effect of the drug on mouse cardiomyocytes.

The present disclosure expands on methods we have developed previously [A Tveito, K H Joger, N Huebsch, B Charrez, Scientific Reports, 8(1):17626, 2018; K H Joger et al., Frontiers in Pharmacology, 10:1648, 2020] for extrapolating drug effects obtained using hiPSC-CMs to healthy, mature human ventricular myocytes. Importantly, we found that embodiments can be used to translate measured pharmacological effects on the action potential in animal models to those in healthy adult human myocytes. An assumption underlying this approach is that major currents are carried by the same or similar ion channels for which isoforms differences have been measured and are relatively well understood. This is indeed often the case. For instance, in epicardial adult ventricular myocytes, channels underpinning late repolarization (delayed rectifier currents rapid and slow, I_{Kr}/hERG and I_{KS}/KvLQT-minK, respectively) are conserved among human, canine, guinea pig, and rabbit cells [N Jost et al., Cardiologia Hungarica, 34:103-113, 2004; S Zicha et al., American Journal of Physiology-Heart and Circulatory Physiology, 285(4):H1641-H1649, 2003]. Again, the transient outward current, Iₜₒ, prominent in early depolarization in the rabbit ventricle via Kv1.4/4.2/4.3, is similar in presentation and action potential effects in adult human myocytes, as carried by Kv4.3 [Wang et al., Circulation Research, 84(5):551-561, 1999]. The inward rectifying K⁺ current (I_{K1}), the L-type Ca²⁺ current (I_{CaL}), and the Na⁺/K⁺ pump current (I_{NaK}) are also present and effectual in rabbit [P Gemmell et al., PLoS One, 9(2):e90112, 2014] in addition to other species, as in human.

Rabbit measurements of calcium handling and excitation-contraction coupling have long been used as a basis to study and model these important phenomenon in human myocytes [E Grandi, F S Pasqualini, and D M Bers, Journal of Molecular and Cellular Cardiology, 48(1):112-121, 2010], and the molecular constituents of all three major depolarizing sarcolemmal currents, I_{CaL}, I_{Na}, and I_{NaCa}, are thought to be relatively consistent across species [A G Edwards and W E Louch, Clinical Medicine Insights: Cardiology, 11:1179546816686061, 2017]. While meaningful functional differences between species have additionally been established, the relative conservation of expressed ion channel proteins across mammalian species provides a solid basis for application of our methodology for translation of drug effects across most large mammals, given an understanding of the divergence, as well as sufficient data for parameterization and validation.

D. Computational Techniques Using Animal Models

1. Animal Models

Mathematical models of the membrane potential of excitable cells can be written on the form

$$\frac{dv}{dt} = - {\sum }_{x} {I}_{x} , \qquad ( 23 )$$

see e.g. [Y Rudy, Heart, Comprehensive Biophysics, pages 268-327, 2012; Y Rudy and JR Silva, Quarterly Reviews of Biophysics, 39(01):57-116, 2006; R Plonsey and R C Barr, Bioelectricity, A Quantitative Approach, Springer, 2007; D Sterratt et al., Principles of Computational Modelling in Neuroscience. Cambridge University Press, 2011]. Here, v is the membrane potential (in mV), t denotes time (in ms) and Iₓ are the membrane currents (in A/F). Each individual current can be written on the form

$${I}_{x} = \frac{{N}_{x}}{A {C}_{m}} {g}_{0}^{x} {o}_{x} ( v - {E}_{x} ) , \qquad ( 24 )$$

where A is the area of the cell membrane (in μm 2) and Cₘ is the specific capacitance of the cell membrane (in pF/μm²); both A and Cₘ are common parameters for each currents in a given type of cardiomyocyte. Furthermore, Nₓ is the number of channels of type x, g₀^{x} is the conductance of a single open channel (in nS), oₓ is the unitless open probability of the channel. Finally, Eₓ is the electrochemical equilibrium potential of the channel (in mV). By introducing the constant

$${ρ}_{x} = \frac{{N}_{x}}{A {C}_{m}} , \qquad ( 25 )$$

we get the simpler expression

Iₓ=ρₓg₀^{x}oₓ(v−Eₓ). (26)

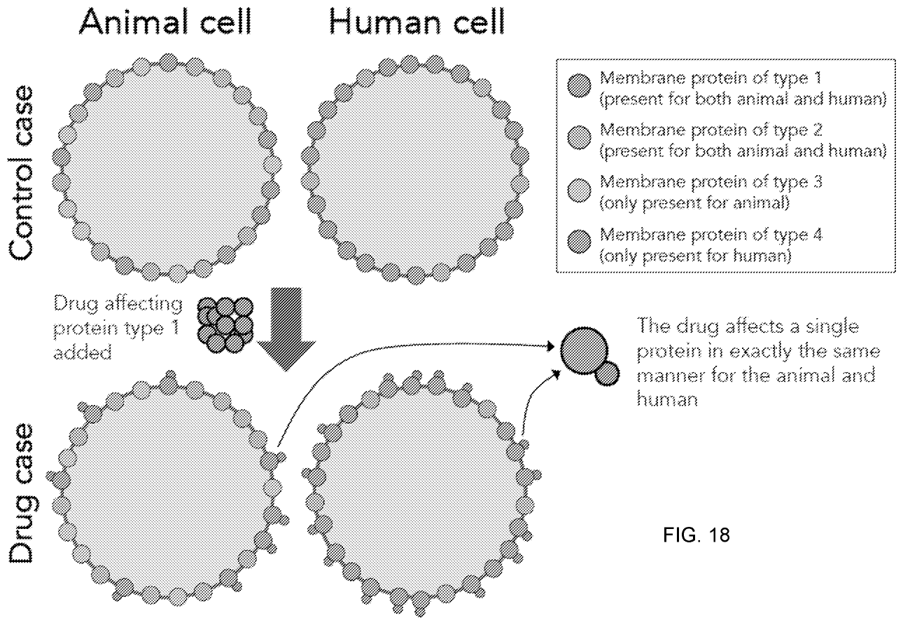

In (26), it is important to note that the characteristics of the dynamics of the specific ion channel under consideration is given by the open probability oₓ; the rest of the expression is either constants or the common term (v−Eₓ). The dynamics of oₓ can be modelled using a Markov scheme (see e.g. [Y Rudy, Heart, Comprehensive Biophysics, pages 268-327, 2012; Y Rudy and JR Silva, Quarterly Reviews of Biophysics, 39(01):57-116, 2006; A Tveito and G T Lines, Computing Characterizations of Drugs for Ion Channels and Receptors Using Markov Models. Springer-Verlag, Lecture Notes, vol. 111, 2016]). Here, it is critical to acknowledge that if the ion channel in two different species are identical, then so is the Markov model governing their dynamics. The diagram in FIG. 18 illustrate the assumptions underlying the translation from animal to human.

The effect of a specific channel blocker is often expressed in terms of an IC₅₀ value (see e.g. [T Brennan, M Fink, and B Rodriguez, European Journal of Pharmaceutical Sciences, 36(1):62-77, 2009]; here we use the Hill coefficient 1). The IC₅₀ value indicates the concentration that reduces the channel conductance by 50%. We assume that when a channel blocker is applied, the current takes the form

Iₓ(D)=ρₓgₓ⁰(D)oₓ(v−Eₓ), (27)

where we have defined the conductance of the single channel to be

$${g}_{x}^{0} ( D ) = \frac{{g}_{0}^{x}}{1 + {ε}_{x} D} . \qquad ( 28 )$$

In (28), we have introduced the parameter εₓ=1/IC₅₀; here D is the drug concentration given in the same unit as the IC₅₀ (often μM). Clearly, Iₓ(IC₅₀)=½Iₓ(0), where Iₓ(0) is the current in baseline or control case. Note from (27) that we assume that the blocker only affects the maximum conductance of the single channel. This is a known oversimplification since the effects of drugs may be much more complex; see e.g. [C E Clancy, Z I Zhu, and Y Rudy, AJP: Heart and Circulatory Physiology, 292(1):H66-H75, 2007; A Tveito, M M Maleckar, and GT Lines, Computational and Mathematical Biophysics, 6(1):41-64, 2018; A Tveito and G T Lines, Computing Characterizations of Drugs for Ion Channels and Receptors Using Markov Models. Springer-Verlag, Lecture Notes, vol. 111, 2016]. However, this assumption greatly simplifies the identification of the drug effect from measurements, and it is sufficient for our purposes.

When a drug effect on an ion channel in an animal myocyte is detected, the key observation for translation to humans is that, at the level of one individual channel protein, the effect of this drug must be the same; that is independent of whether the protein is expressed in an animal or human. Accordingly, the IC₅₀ value is equal for human and animal single channels: that is the crux of the translation of the effect of the drug. In principle, this key observation also is valid when the drug effect is modeled as a part of a complex Markov model, since the effect of the drug, on the single channel, again would be the same for animals and humans as long as the single ion channels are identical.

Let us next consider a very simple situation where we assume that both the animal myocyte action potential (a) and the human action potential (h) are generated by only two currents. The mathematical models takes then the form

$$\frac{d {v}^{y}}{d t} = - {ρ}_{1}^{y} \frac{{g}_{0}^{1}}{1 + {ε}_{1} D} {o}_{1}^{y} ( {v}^{y} - {E}_{1} ) - {ρ}_{2}^{y} \frac{{g}_{0}^{2}}{1 + {ε}_{2} D} {o}_{2}^{y} ( {v}^{y} - {E}_{2} ) , \qquad ( 29 )$$

where y is either a for animal or h for human. Here, clearly ρ₁^{y}, ρ₂^{y} are different for y=a and y=h due to differences in the ion channel density. However, since the ion channels are the same, the Markov model governing the of, of for y=a and y=h are the same.

Suppose now that we have measured an animal dataset sufficient to determine the coefficients ε₁ and ε₂. We immediately observe that we have a complete mathematical model for both the animal and the human action potentials. Using these principles, we can compute the biomarkers of interest for the human case.

Certainly, mathematical models used to represent action potentials are much more complex than the template model given by (29); see e.g. [D C Kernik et al., The Journal of Physiology, 2019; E Grandi, F S Pasqualini, and DM Bers, Journal of Molecular and Cellular Cardiology, 48(1):112-121, 2010; T O'Hara, L Virig, A Varri, and Y Rudy, PLoS Computational Biology, 7(5):e1002061, 2011]. In our computations, we use the Base model developed in [K H Joger et al., Frontiers in Pharmacology, 10:1648, 2020], as it systematically uses the same model for the identical ion channel in two different species.

### 2. Example

In order to explain and illustrate how it is possible to use animal data to gain quantitative insight concerning effects on human ventricular myocytes, we will present a very simple example. Let us for the sake of argument assume that an animal myocyte action potential is governed by

$$\frac{d v}{d t} = - {g}_{1} {I}_{N a} - {g}_{2} {I}_{C a L} - {g}_{3} {I}_{K s}$$

and the analogous human AP is governed by the very simple model,

$$\frac{d v}{d t} = - {k}_{1} {I}_{N a} - {k}_{2} {I}_{C a L} - {k}_{3} {I}_{K r} .$$

Here g₁, g₂, g₃, k₁, k₂, k₃ are conductances and we assume that they are of significant magnitude. Suppose, for instance, that a drug is applied to the animal ventricular myocytes and inversion of the measured data suggests that the drug is a calcium blocker with the IC₅₀ value given by 1/ε_{CaL}. Then the animal model in the setting when the drug has been applied is given by

$$\frac{d v}{d t} = - {g}_{1} {I}_{N a} - \frac{{g}_{2}}{1 + D {ε}_{C a L}} {I}_{C a L} - {g}_{3} {I}_{Ks} ,$$

where D denotes the concentration of the applied drug. Since the IC₅₀ value of the single channel is the same for the animal and the human myocytes, the human model takes the form

$$\frac{d v}{d t} = - {k}_{1} {I}_{N a} - \frac{{k}_{2}}{1 + D {ε}_{C a L}} {I}_{C a L} - {k}_{3} {I}_{K r} .$$

This model can be used to compute chosen biomarkers of interest for the human action potential. In this particularly simple situation, we can conclude that:
1. One can use the animal datasets to reveal drug effect on human I_{Na} and I_{CaL}.
2. One cannot use the animal to get any information about human I_{Kr} because the animal does not have I_{Kr}.
3. The I_{Ks} current in the animal does not create difficulties for the measurements.

TABLE 7
Parameter values of the Base model used to represent different species.
The remaining parameter values used in the simulations are as specified in
[KH Jasger et al., Frontiers in Pharmacology, 10:1648, 2020].

| Parameter | Human | Dog | Zebrafish | Rabbit |
|---|---|---|---|---|
| g_{Kr} (mS/μF) | 0.025 | 0.011 | 0.044 | 0.066 |
| g_{CaL} (nL/(μF ms)) | 0.12 | 0.079 | 0.076 | 0.26 |
| g_{Na} (mS/μF) | 5 | 1.8 | 2.6 | 2.3 |
| g_{Ks} (mS/μF) | 0.003 | 0.069 | 0.0033 | 0.023 |
| g_{K1} (mS/μF) | 0.074 | 0.4 | 0.64 | 0.65 |
| gₜₒ (mS/μF) | 0.38 | 0.35 | 0.0038 | 0.18 |
| g_{NaL} (mS/μF) | 0.025 | 0.0092 | 0.031 | 0.024 |
| Ī_{NaCa} (μA/μF) | 4.9 | 8 | 2.2 | 11 |
| Ī_{NaK} (μA/μF) | 1.8 | 2.6 | 5.6 | 0.65 |
| g_{bCl} (mS/μF) | 0.007 | 0.00021 | 0.0042 | 0.019 |
| g_{bCa} (mS/μF) | 0.00055 | 0.00044 | 0.00081 | 0.00049 |
| Ī_{pCa} (μA/μF) | 0.068 | 0.42 | 0.061 | 0.047 |
| g_{CaT} (mS/μF) | 0 | 0 | 0.082 | 0 |

3. The Base Model of the Action Potential

In all of our simulations, we use the base model derived in [K H Joger et al., Frontiers in Pharmacology, 10:1648, 2020]. The parameters used to model human, dog, zebrafish and rabbit action potentials are given in Table 7. The remaining parameter values are specified in [K H Joger et al., Frontiers in Pharmacology, 10:1648, 2020]. Note also that because zebrafish cardiomyocytes have been shown to exhibit a robust T-type calcium current (I_{CaT}) [P Nemtsas et al., Journal of Molecular and Cellular Cardiology, 48(1):161-171, 2010], the Base model from [K H Joger et al., Frontiers in Pharmacology, 10:1648, 2020], when utilized for the zebrafish simulations, is extended to include a T-type calcium current from [V A Maltsev and E G Lakatta, American Journal of Physiology-Heart and Circulatory Physiology, 296(3):H594-H615, 2009] of the form

I_{CaT}=g_{CaT}·d·f·(v−E_{Ca}),

where g_{CaT} is the conductance of the channels and E_{Ca} is the Nernst equilibrium potential for calcium (see [K H Joger et al., Frontiers in Pharmacology, 10:1648, 2020]). Here, d and f are gating variables governed by

$$\frac{d d}{d t} = \frac{{d}_{∞} - d}{{τ}_{d}} , {d}_{∞} = \frac{1}{1 + {e}^{- ( v + 26.3 ) / 6}} , \\ {τ}_{d} = \frac{1}{1.068 {e}^{( v + 26.3 ) / 30} + 1.068 {e}^{- ( v + 26.3 ) / 30}} , \\ \frac{d f}{d t} = \frac{{f}_{∞} - f}{{τ}_{f}} , {f}_{∞} = \frac{1}{1 + {e}^{( v + 61.7 ) / 5.6}} , \\ {τ}_{f} = \frac{1}{0.0153 {e}^{- ( v + 61.7 ) / 83.3} + 0.015 {e}^{( v + 61.7 ) / 15.38}} .$$

4. Inversion of data

The inversion of data from dog, zebrafish and rabbit action potentials is performed using the inversion procedure described in [K H Joger et al., Frontiers in Pharmacology, 10:1648, 2020]. In the inversion, the adjustment factors λ_{Kr}, λ_{CaL}, λ_{Na}, λ_{Ks}, λ_{K1}, λₜₒ, λ_{NaL}, λ_{NaCa}, λ_{NaK}, λ_{bCl}, λ_{bCa}, and λ_{pCa} for the currents I_{Kr}, I_{CaL}, I_{Na}, I_{Ks}, I_{K1}, Iₜₒ, I_{NaL}, I_{NaCa}, I_{NaK}, I_{bCl}, I_{bCa}, and I_{pCa}, in addition to the drug effect parameter ε_{Kr}, are treated as free parameters¹. ¹In the zebrafish case, the adjustment factor λ_{CaT} for the current I_{CaT} is also treated as a free parameter in the inversion.

In the inversion, we minimize a cost function of the form

H(λ,ε)=Σ_{d}Σⱼw_{d,j}(Hⱼ(λ,ε,D_{d}))², (30)

where d numbers the drug doses, D_{d}, included in the data set (including the control case), j numbers the different cost function terms, Hⱼ, included in the cost function, and w_{d,j} are specified weights for each of the cost function terms and each of the doses. The cost function includes the terms

$${H}_{A P D p} ( λ , ε , {D}_{d} ) = \frac{\vert APD p ( λ , ε , {D}_{d} ) - A P D {p}^{*} ( {D}_{d} ) \vert}{\vert APD {p}^{*} ( {D}_{d} ) \vert} , \\ for p = 20 , 30 , … , 90 , {H}_{A P A} ( λ , ε , {D}_{d} ) = \frac{\vert AP A ( λ , ε , {D}_{d} ) - A P {A}^{*} ( {D}_{d} ) \vert}{\vert {APA}^{*} ( {D}_{d} ) \vert} , {H}_{{v}_{max}} ( λ , ε , {D}_{d} ) = \frac{\vert {v}_{max} ( λ , ε , {D}_{d} ) - {v}_{max}^{*} ( {D}_{d} ) \vert}{\vert {v}_{max}^{*} ( {D}_{d} ) \vert} , {H}_{{v}_{min}} ( λ , ε , {D}_{d} ) = \frac{\vert {v}_{min} ( λ , ε , {D}_{d} ) - {v}_{min}^{*} ( {D}_{d} ) \vert}{\vert {v}_{min}^{*} ( {D}_{d} ) \vert} ,$$

where APDp is the action potential duration at p percent repolarization (see [K H Joger et al., Frontiers in Pharmacology, 10:1648, 2020]). Furthermore, APA is the action potential amplitude, and vₘᵢₙ and vₘₐₓ are the minimum and maximum values of the membrane potential, respectively. The terms marked by an * represent values computed from measured data, and the remaining terms are computed from simulations of the model defined by A and E.

In some implementations, we use the weight 2 for H_{APA}, Hᵥ_{ₘₐₓ}, Hᵥ_{ₘᵢₙ} and H_{APD50}, the weight 5 for H_{APD90} and the weight 1 for the remaining terms. In addition, the weights for the control case, w_{0,j}, are multiplied by the total number of doses included in the data set (including the control case).

In some implementations, in order to minimize the cost function (30), we apply the continuation-based minimization procedure from [K H Joger et al., Frontiers in Pharmacology, 10:1648, 2020] using 20 iterations with 100 randomly chosen initial guesses. The initial guesses for A are chosen within 10% above or below the optimal values from the previous iteration, and the initial guesses for εₘ are chosen within [εₘ₋₁/5.5εₘ₋₁], where εₘ₋₁ is the optimal ε from the previous iteration. From these initial guesses we run 15 or 30 iterations of the Nelder-Mead algorithm [J A Nelder and R Mead, The Computer Journal, 7(4):308-313, 1965], for the first fifteen and the last five continuation iterations, respectively. The starting points for the first iteration of the continuation method are adjusted by hand.

E. Application of the Method to Generated Data

In the main text, we have shown that we are able to use animal datasets (from dog, zebrafish and rabbit) to estimate the effect of I_{Kr} blockers on healthy human ventricular myocytes. However, available published data is limited and we therefore want to indicate that the methodology works more generally than we have been able to prove using available datasets. In Table 8, we have additionally used simulations to generate data. More specifically, we use the zebrafish model to estimate the effect for the human model of blocking the sodium current and the calcium current by 50%. In these inversions, λ_{Na}, λ_{CaL} and λ_{Kr} and ε_{Na}, ε_{CaL} and ε_{Kr} are treated as free parameters. In the generation of the zebrafish data, I_{Na}, I_{CaL}, I_{Kr} are all increased by 20%. Furthermore, in order to detect changes in the sodium current, we extend the cost function to include the term

$${H}_{d v d t} ( λ , ε , {D}_{d} ) = \frac{\vert \frac{d v ( λ , ε , {D}_{d} )}{d t} - \frac{d {v}^{*} ( {D}_{d} )}{d t} \vert}{\vert \frac{d {v}^{*} ( {D}_{d} )}{d t} \vert} ,$$

measuring differences in the upstroke velocity. The results in Table 8 illustrate that the method presented here has wide applicability, provided that the ion currents under consideration have significant impact on both animal and human action potentials.

TABLE 8

| | | | | | | Human drug, |
|---|---|---|---|---|---|---|
| | Zebrafish | | | | Human | estimated |
| | control | Zebrafish drug | Human control | Human drug | control | based on |
| | “measured” | “measured” | “measured” | “measured” | model | zebrafish data |
| APD50 (ms) | 109 | −9.8% | 214 | −10.5% | 214 | −9.3% |
| (dv/dt) max | 106 | −50.9% | 195 | −43.6% | 195 | −43.2% |
| (mV/ms) | | | | | | |

Effect of 50% block of I_{Na} and I_{CaL} on APD50 and the maximal upstroke velocity of the action potential in zebrafish and human models. The zebrafish and human data is generated from simulations of the zebrafish and human models specified in Section 4.2. In addition, the effect of the drug is estimated based on the zebrafish case and mapped to the human case in the rightmost column.

F. Method Using Animal Cell

The results above show that the effect of a drug as measured for animal cardiomyocytes (CMs) can be used to determine the effect of the drug on human CMs. Such a technique allows animal cells to be used instead of human cells for such testing.

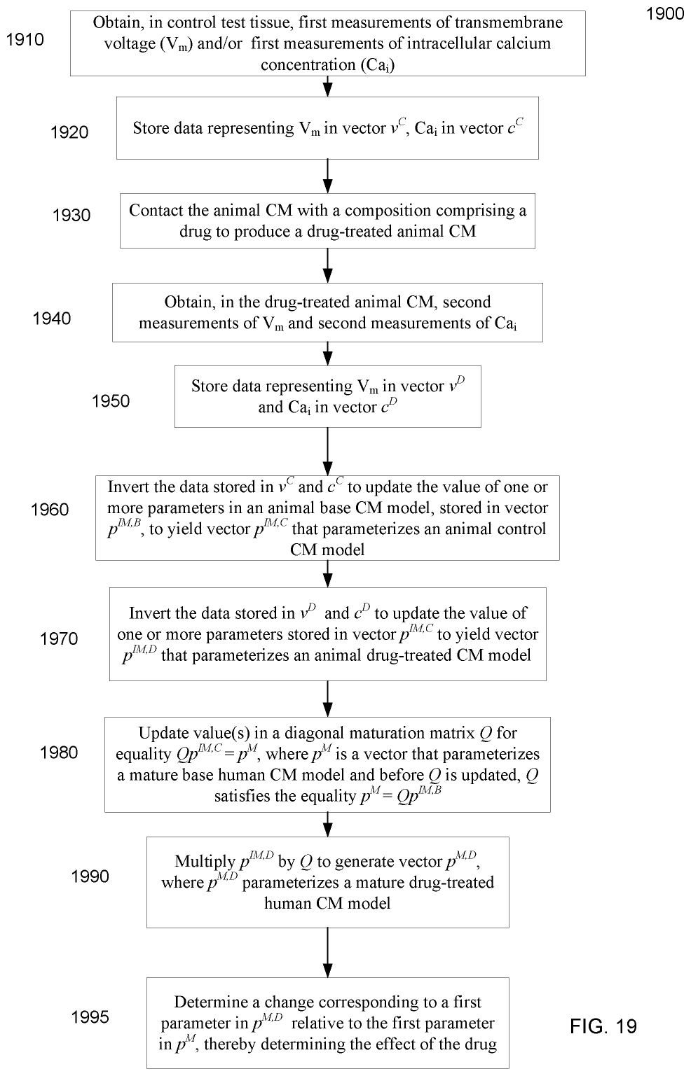

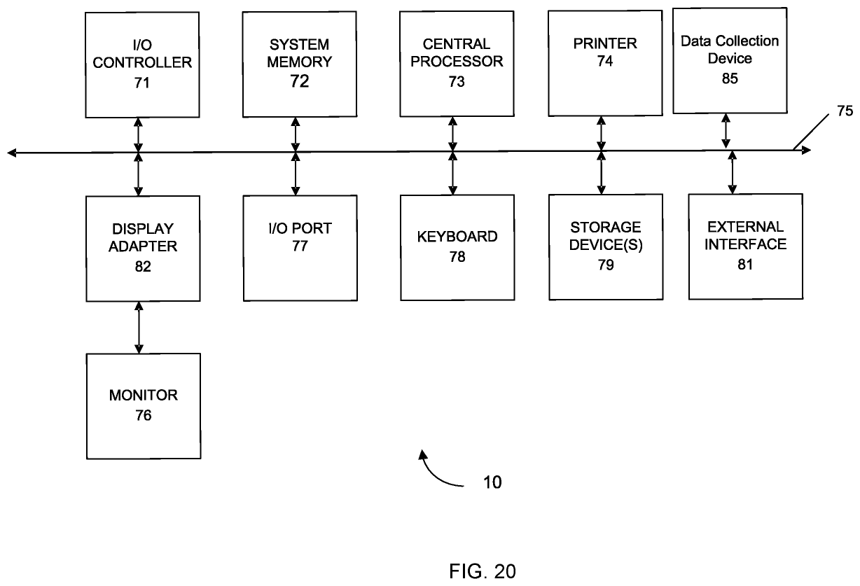

FIG. 19 is a flowchart of a method 1900 for determining an effect of a drug on a mature cardiomyocyte (CM) using measurements from animal CMs according to embodiments of the present disclosure. Aspects of method 1900 can be performed in a similar manner as corresponding steps of method 1500.

At block 1910, one or more first measurements of transmembrane voltage (Vₘ) and/or one or more first measurements of intracellular calcium concentration (Caᵢ) are obtained in an animal CM that is from an animal that is not human. The measurements of Vₘ and Caᵢ can be optical measurements, e.g., as described herein. In addition to these measurements, some embodiments can also measure an extracellular potential (U) when animal tissue of multiple animal cells is used. The measurements can correspond to a time series of measurement of a portion or all of a cycle of a heartbeat.

At block 1920, data representing said one or more first measurements of Vₘ are stored in vector v^{C}, and/or sdata representing said one or more first measurements of Caᵢ are stored in vector c^{C}. Block 1920 can be performed in a similar manner as block 1520 in FIG. 15.

At block 1930, the animal CM is contacted with a composition comprising a sufficient amount of the drug to produce a drug-treated animal CM. The amount of drug can correspond to a different doses that might be expected to be used for the particular type of animal whose CM is being used. The drug can be designed to treat any number of diseases, i.e., not necessarily heart related. Block 1930 can be performed in a similar manner as block 1530 in FIG. 15.

At block 1940, one or more second measurements of Vₘ and/or one or more second measurements of Caᵢ are obtained in the drug-treated animal CM. The second measurements may be performed in a same manner as the first measurements. Block 1940 can be performed in a similar manner as block 1540 in FIG. 15.

At block 1950, data representing said one or more second measurements of Vₘ are stored in vector v^{D}, and/or data representing said one or more second measurements of Caᵢ are stored in vector c^{D}. The data can be of a same type as measured in block 1920.

At block 1960, the data stored in v^{C} and/or c^{C} are inverted to update the value of one or more parameters in an animal base CM model (which is stored in vector p^{IM,B}) to yield vector p^{IM,C} that parameterizes an animal control CM model. When the extracellular potential U is also measured, then inversion techniques described in section II can be used. However, if the measurements are only for a cell, e.g., the transmembrane voltage V and the cytosolic calcium concentration Ca, then other inversion techniques in section VI.D can be used.

At block 1970, the data stored in v^{D} and/or c^{D} are inverted to update the value of one or more parameters stored in vector p^{IM,C} to yield vector p^{IM,D} that parameterizes an animal drug-treated CM model. The inversion may be performed in a similar manner as in block 1960, but using the data for the second measurements. The drug-treated model and the base model can have a same functional structure, but with the drug-treated tissue model being trained using the drug measurements. The inversion in blocks 1960 and 1970 can be performed simultaneously.

At block 1980, one or more values in a diagonal maturation matrix Q are updated such that the equality Qp^{IM,C}=p^{M} is satisfied, where p^{M} is a vector that parameterizes a mature base human CM model. Before Q is updated, Q satisfies the equality p^{M}=Qp^{IM,B}. The maturation map can be updated to secure that if Q is applied to the IM parameter vector, p^{IM,C}, the resulting parameter vector is the base model of the M cell parameterized by the vector p^{M}. Examples of updating Q are provided in section V.D, describing method 1500.

At block 1990, p^{IM,D} can be multiplied by Q to generate vector p^{M,D}, where p^{M,D} parameterizes a mature drug-treated human CM model. In this manner, a mature drug-treated human CM model can be determined using measurements from animal cell(s).

At block 1995, a change corresponding to a first parameter in the vector p^{M,D} relative to the first parameter in the vector p^{M} is determined. The change can provide the effect of the drug. The change can be used to determine whether the drug is safe. If the change is too high, then the drug can be deemed not safe. Block 1995 can be performed in a similar manner as block 1980 in FIG. 15.

Similar to method 1500, the determination of the change can include using the mature base human CM model and the mature drug-treated human CM model to generate a simulated mature control action potential (AP) and a simulated mature drug-treated AP, respectively; and comparing one or more morphological properties of the simulated mature control AP and the simulated mature drug-treated AP in order to calculate a difference.

In various embodiments, only v^{C} and v^{D} are inverted; only c^{C} and C^{D} are inverted; and v^{C}, c^{C}, v^{D}, and C^{D} are inverted. In some embodiments, wherein when v^{D} or C^{D} are inverted, the simulated drug effect is that of reducing the maximum conductance of an ion channel or the maximum rate of an ion pump or ion exchanger.

### VII. SIMULTANEOUS INVERSION

In some embodiments, the inversion of the control data (first measurements) and the drug effect data (second measurements) to determine (solve) the control tissue model and the drug-treated tissue model can be performed simultaneously. The drug effect can be defined in terms of the blocking factors. Such a process is described below for a model that does not include extracellular potential (U), but based on the description herein, the skilled person will appreciate how to perform such a simultaneous (coupled) inversion for embodiments using the extracellular potential (U) and/or using animal cells.

### A. Modeling of Drug Effects

Following previous modeling of channel blockers (see e.g., Michelangelo Paci et al., British Journal of Pharmacology, 172(21):5147-5160, 2015; Thomas Brennan, Martin Fink, and Blanca Rodriguez, European Journal of Pharmaceutical Sciences, 36(1):62-77, 2009; Mark Ri Davies et al., American Journal of Physiology-Heart and Circulatory Physiology, 2012; Nejib Zemzemi et al., British Journal of Pharmacology, 168(3):718-733, 2013), we model the dose-dependent effect of a drug by scaling the channel conductances according to

$${g}_{i}^{D} = \frac{1}{1 + \frac{D}{IC {50}_{i}}} {g}_{i}^{C} , \qquad ( 31 )$$

where gᵢ^{D} is the conductance of channel i in the presence of a drug with concentration D, IC50 is the drug concentration that leads to 50% block of channel i, and gᵢ^{C} is the channel conductance in the control case (i.e., in the absence of drugs). Specifically, this means that if the drug concentration D equals the IC50 value, we have gᵢ^{D}=½gᵢ^{C}

It should be mentioned that a drug may certainly affect a channel in a more complex manner than is assumed here. The effect of drugs can realistically be represented by introducing new states in Markov models of the ion channel. In such models, the transition rates between different model states are able to represent the properties of drugs (see e.g., Colleen E Clancy, Zheng I Zhu, and Yoram, AJP: Heart and Circulatory Physiology, 292(1):H66-H75, 2007; Aslak Tveito et al., Mathematical Biosciences and Engineering, 8(3):861-73, 2011; Aslak Tveito and Glenn T Lines, Computing Characterizations of Drugs for Ion Channels and Receptors Using Markov Models, Springer-Verlag, Lecture Notes, vol. 111, 2016; Aslak Tveito, Mary M Maleckar, and Glenn T Lines, Computational and Mathematical Biophysics, 6(1):41-64, 2018). Although Markov model representations of drug effects are more versatile and realistic than the simple blocking assumption employed here (31), it would greatly increase the complexity of the inversion process, as many more parameters would have to be computed.

From (31), we see that for a given drug dose D>0, the effect of the drug would increase if the IC50 value were decreased, and the effect of the drug would be very small if the IC50 value were much larger than the considered dose. In the continuation-based minimization method applied in our computations (see Section 2.4.6 below), it is most practical to deal with parameters that are small when no change occurs and large when large changes occur. Therefore, we introduce the parameters

$${ε}_{i} = \frac{1}{I C 5 {0}_{i}} . \qquad ( 32 )$$

Here, a small value of E, represents small effects of a drug while a large value of εᵢ represents large effects, and channel blocking is given by

$${g}_{i}^{D} = \frac{1}{1 + D {ε}_{i}} {g}_{i}^{C} . \qquad ( 33 )$$

In our computations, we assume that the considered drugs block either I_{CaL}, I_{NaL}, I_{Kr}, or a combination of these currents, and we therefore only consider the ε-parameters ε_{CaL}, ε_{NaL},

### B. Coupled Inversion of Data from Several Doses

The control data obtained from different optical experiments tend to vary significantly, and in order to be able to accurately estimate the drug effect from these measurements, the λ—parameters must be tuned so that the control model fits the control data as best possible. In addition, we want the λ parameters to be constructed such that that the scaling (33) for ε_{CaL}, ε_{NaL}, and ε_{Kr} is sufficient to fit the model to the drug doses under consideration. In order to increase the chance of obtaining such a control model, we fit the control parameters, λ, and the drug parameters, ε, simultaneously, instead of first finding the optimal control parameters, λ, by fitting the base model to the control data, and then subsequently finding appropriate drug parameters, ε, for each dose. In addition, all doses are included in the inversion, so that the estimated values of ε are based on all the drug doses included in the data set.

In order to illustrate the role of the λ- and ε-parameters more clearly, consider a simplified model consisting of just two currents, and assume that the base model is given by:

$$\frac{d v}{d t} = - {g}_{1} {o}_{1} ( v - {E}_{1} ) - {g}_{2} {o}_{2} ( v - {E}_{2} ) . \qquad ( 34 )$$

Assume further that we have data from cells with both no drug present and with different doses of a drug (e.g., one low dose and one high dose). We assume that the drug may block any of the two model currents. In the inversion procedure, we try to find optimal values of the four parameters λ₁, λ₂, ε₁ and ε₂ so that the adjusted model of the form

$$\frac{d v}{d t} = - \frac{1 + {λ}_{1}}{1 + D {ε}_{i}} {g}_{1} {o}_{1} ( v - {E}_{1} ) - \frac{1 + {λ}_{2}}{1 + D {ε}_{i}} {g}_{2} {o}_{1} ( v - {E}_{2} ) \qquad ( 35 )$$

fits the data both for the control case (D=0) and for the considered drug doses. In other words, for a given parameter set λ₁, λ₂, ε₁ and ε₂, we need to compute the solution of the model (35) both for the control case (D=0) and for the considered drug doses and compare the obtained solutions to the corresponding experimental data.

The more general case considered in our computations is conceptually identical; however, as we also consider scaling of parameters that are not assumed to possibly be affected by the drug, we also have some parameters simply scaled by a factor (1+λᵢ) instead of by

$$\frac{1 + {λ}_{f}}{1 + D {ε}_{i}} .$$

### C. Example Properties of the Cost Function

In order to find the optimal parameters for fitting the model to data, we need to define a cost function that measures the difference between a given model solution and the data. This cost function is defined as

$$H ( λ , ε ) = {\sum }_{d} {\sum }_{j} {{w}_{d , j} ( {H}_{j} ( λ , ε , {D}_{d} ) )}^{2} . \qquad ( 36 )$$

Here, d runs over each of the considered drug doses, D_{d}, including the control case (D₀=0), and j runs over each cost function term, Hⱼ, representing various differences between the data and the model solution. The parameters w_{d,j} represent weights for each of the cost function terms for each of the doses. Each of the cost function terms, Hⱼ, can be defined as described herein.

Further details of such implementation can be found in Jaeger K H, et al., “Improved Computational Identification of Drug Response Using Optical Measurements of Human Stem Cell Derived Cardiomyocytes in Microphysiological Systems,” Front Pharmacol. 2020 Feb. 12; 10:1648.

### VIII. COMPUTER SYSTEM

Any of the computer systems mentioned herein may utilize any suitable number of subsystems. Examples of such subsystems are shown in FIG. 19 in computer system 10. In some embodiments, a computer system includes a single computer apparatus, where the subsystems can be the components of the computer apparatus. In other embodiments, a computer system can include multiple computer apparatuses, each being a subsystem, with internal components. A computer system can include desktop and laptop computers, tablets, mobile phones and other mobile devices.

The subsystems shown in FIG. 19 are interconnected via a system bus 75. Additional subsystems such as a printer 74, keyboard 78, storage device(s) 79, monitor 76 (e.g., a display screen, such as an LED), which is coupled to display adapter 82, and others are shown. Peripherals and input/output (I/O) devices, which couple to I/O controller 71, can be connected to the computer system by any number of means known in the art such as input/output (I/O) port 77 (e.g., USB, FireWire®). For example, I/O port 77 or external interface 81 (e.g. Ethernet, Wi-Fi, etc.) can be used to connect computer system 10 to a wide area network such as the Internet, a mouse input device, or a scanner. The interconnection via system bus 75 allows the central processor 73 to communicate with each subsystem and to control the execution of a plurality of instructions from system memory 72 or the storage device(s) 79 (e.g., a fixed disk, such as a hard drive, or optical disk), as well as the exchange of information between subsystems. The system memory 72 and/or the storage device(s) 79 may embody a computer readable medium. Another subsystem is a data collection device 85, such as a camera, microphone, accelerometer, and the like. Any of the data mentioned herein can be output from one component to another component and can be output to the user.

A computer system can include a plurality of the same components or subsystems, e.g., connected together by external interface 81, by an internal interface, or via removable storage devices that can be connected and removed from one component to another component. In some embodiments, computer systems, subsystem, or apparatuses can communicate over a network. In such instances, one computer can be considered a client and another computer a server, where each can be part of a same computer system. A client and a server can each include multiple systems, subsystems, or components.

Aspects of embodiments can be implemented in the form of control logic using hardware circuitry (e.g. an application specific integrated circuit or field programmable gate array) and/or using computer software with a generally programmable processor in a modular or integrated manner. As used herein, a processor can include a single-core processor, multi-core processor on a same integrated chip, or multiple processing units on a single circuit board or networked, as well as dedicated hardware. Based on the disclosure and teachings provided herein, a person of ordinary skill in the art will know and appreciate other ways and/or methods to implement embodiments of the present invention using hardware and a combination of hardware and software. Further, measurements can be performed by robotics controlled by computer systems according to embodiments of the present disclosure.

Any of the software components or functions described in this application may be implemented as software code to be executed by a processor using any suitable computer language such as, for example, Java, C, C++, C#, Objective-C, Swift, or scripting language such as Perl or Python using, for example, conventional or object-oriented techniques. The software code may be stored as a series of instructions or commands on a computer readable medium for storage and/or transmission. A suitable non-transitory computer readable medium can include random access memory (RAM), a read only memory (ROM), a magnetic medium such as a hard-drive or a floppy disk, or an optical medium such as a compact disk (CD) or DVD (digital versatile disk) or Blu-ray disk, flash memory, and the like. The computer readable medium may be any combination of such storage or transmission devices.

Such programs may also be encoded and transmitted using carrier signals adapted for transmission via wired, optical, and/or wireless networks conforming to a variety of protocols, including the Internet. As such, a computer readable medium may be created using a data signal encoded with such programs. Computer readable media encoded with the program code may be packaged with a compatible device or provided separately from other devices (e.g., via Internet download). Any such computer readable medium may reside on or within a single computer product (e.g. a hard drive, a CD, or an entire computer system), and may be present on or within different computer products within a system or network. A computer system may include a monitor, printer, or other suitable display for providing any of the results mentioned herein to a user.

Any of the methods described herein may be totally or partially performed with a computer system including one or more processors, which can be configured to perform the steps. Thus, embodiments can be directed to computer systems configured to perform the steps of any of the methods described herein, potentially with different components performing a respective step or a respective group of steps. Although presented as numbered steps, steps of methods herein can be performed at a same time or at different times or in a different order. Additionally, portions of these steps may be used with portions of other steps from other methods. Also, all or portions of a step may be optional. Additionally, any of the steps of any of the methods can be performed with modules, units, circuits, or other means of a system for performing these steps.

The specific details of particular embodiments may be combined in any suitable manner without departing from the spirit and scope of embodiments of the invention. However, other embodiments of the invention may be directed to specific embodiments relating to each individual aspect, or specific combinations of these individual aspects.

The above description of example embodiments of the present disclosure has been presented for the purposes of illustration and description. It is not intended to be exhaustive or to limit the disclosure to the precise form described, and many modifications and variations are possible in light of the teaching above.

The use of “or” is intended to mean an “inclusive or,” and not an “exclusive or” unless specifically indicated to the contrary. Reference to a “first” component does not necessarily require that a second component be provided. Moreover, reference to a “first” or a “second” component does not limit the referenced component to a particular location unless expressly stated. The term “based on” is intended to mean “based at least in part on.”

All patents, patent applications, publications, and descriptions mentioned herein are incorporated by reference in their entirety for all purposes. None is admitted to be prior art.

### IX. REFERENCES

[1] Aslak Tveito, Karoline H Jaeger, Nathaniel Huebsch, Berenice Charrez, Andrew G Edwards, Samuel Wall, and Kevin E Healy. Inversion and computational maturation of drug response using human stem cell de-rived cardiomyocytes in microphysiological systems. Scientific Reports, 8(1):17626, 2018.
[2] Karoline Horgmo Jaeger, Verena Charwat, Berenice Charrez, Henrik Finsberg, Mary M Maleckar, Sam Wall, Kevin Healy, and Aslak Tveito. Improved computational identication of drug response using optical measurements of human stem cell derived cardiomyocytes in microphysiological systems. bioRxiv, page 787390, 2019.
[3] Anurag Mathur, Peter Loskill, Kaifeng Shao, Nathaniel Huebsch, SoonGweon Hong, Sivan G Marcus, Natalie Marks, Mohammad Mandegar, Bruce R Conklin, Luke P Lee, and Kevin E Healy. Human iPSC-based cardiac microphysiological system for drug screening applications. Scientific Reports, 5:8883, 2015.
[4] Anurag Mathur, Zhen Ma, Peter Loskill, Shaheen Jeeawoody, and Kevin E Healy. In vitro cardiac tissue models: current status and future prospects. Advanced Drug Delivery Reviews, 96:203-213, 2016.
[5] James Kramer, Carlos A Obejero-Paz, Glenn Myatt, Yuri A Kuryshev, Andrew Bruening-Wright, Joseph S Verducci, and Arthur M Brown. MICE models: superior to the HERG model in predicting Torsade de Pointes. Scientific Reports, 3:2100, 2013.
[6] Antonella Di Stilo, Sonja Visentin, Clara Cena, Andrea Marcello Gasco, Giuseppe Ermondi, and Alberto Gasco. New 1, 4-dihydropyridines conjugated to furoxanyl moieties, endowed with both nitric oxide-like and calcium channel antagonist vasodilator activities. Journal of Medicinal Chemistry, 41(27):5393-5401, 1998.
[7] William J Crumb, Jose Vicente, Lars Johannesen, and David G Strauss. An evaluation of 30 clinical drugs against the comprehensive in vitro proarrhythmia assay (CiPA) proposed ion channel panel. Journal of Pharmacological and Toxicological Methods, 81:251-262, 2016. Focused Issue on Safety Pharmacology.
[8] Gary R Mirams, Yi Cui, Anna Sher, Martin Fink, Jonathan Cooper, Bronagh M Heath, Nick C McMahon, David J Gavaghan, and Denis Noble. Simulation of multiple ion channel block provides improved early prediction of compounds' clinical torsadogenic risk. Cardiovascular Research, 91(1):53-61, 2011.
[9] Pavel Zhabyeyev, Sergiy Missan, Stephen E Jones, and Terence F Mc-Donald. Low-affinity block of cardiac K⁺ currents by nifedipine. European Journal of Pharmacology, 401(2):137-143, 2000.
[10] Shetuan Zhang, Zhengfeng Zhou, Qiuming Gong, Jonathan C Makielski, and Craig T January. Mechanism of block and identification of the verapamil binding domain to HERG potassium channels. Circulation Research, 84(9):989{998, 1999.
[11] Saeed Mohammad, Zhengfeng Zhou, Qiuming Gong, and Craig T January. Blockage of the HERG human cardiac K⁺ channel by the gastroin-intestinal prokinetic agent cisapride. American Journal of Physiology-Heart and Circulatory Physiology, 273(5):H2534-H2538, 1997.
[12] Limor Zwi, Oren Caspi, Gil Arbel, Irit Huber, Amira Gepstein, In-Hyun Park, and Lior Gepstein. Cardiomyocyte di erentiation of human induced pluripotent stem cells. Circulation, 120(15):1513, 2009.
[13] Mike Clements and Nick Thomas. High-throughput multi-parameter profiling of electrophysiological drug effects in human embryonic stem cell derived cardiomyocytes using multi-electrode arrays. Toxicological Sciences, 140(2):445-461, 2014.
[14] Eliott Tixier, Fabien Raphel, Damiano Lombardi, and Jean-Frederic Gerbeau. Composite biomarkers derived from micro-electrode array measurements and computer simulations improve the classification of drug-induced channel block. Frontiers in physiology, 8:1096, 2018.
[15] Keiichi Asakura, Seiji Hayashi, Atsuko Ojima, Tomohiko Taniguchi, Norimasa Miyamoto, Chiaki Nakamori, Chiho Nagasawa, Tetsuo Kitamura, Tomoharu Osada, Yayoi Honda, et al. Improvement of acquisition and analysis methods in multi-electrode array experiments with ips cell-derived cardiomyocytes. Journal of pharmacological and toxicological methods, 75:17-26, 2015.
[16] Julien Bouyssier and Nejib Zemzemi. Parameters estimation approach for the mea/hipsc-cm asaays. In 2017 Computing in Cardiology (CinC), pages 1-4. IEEE, 2017.
[17] Piero C Franzone, Luca F Pavarino, and Simone Scacchi. Mathematical cardiac electrophysiology, volume 13. Springer, 2014.
[18] Emanuela Abbate, Muriel Boulakia, Yves Coudiere, Jean-Frederic Gerbeau, Philippe Zitoun, and Nejib Zemzemi. In silico assessment of the effects of various compounds in mea/hipsc-cm assays: modeling and numerical simulations. Journal of pharmacological and toxicological methods, 89:59-72, 2018.
[19] Fabien Raphel, Muriel Boulakia, Nejib Zemzemi, Yves Coudiere, Jean-Michel Guillon, Philippe Zitoun, and Jean-Frederic Gerbeau. Identification of ion currents components generating potential recorded in mea from hipsc-cm. IEEE Transactions on Biomedical Engineering, 65(6):1311-1319, 2017.
[20] Fabien Raphel, Tessa de Korte, Damiano Lombardi, Stefan Braam, and Jean-Frédéric Gerbeau. A greedy classifier optimisation strategy to assess ion channel blocking activity and pro-arrhythmia in hipsc-cardiomyocytes. 2019.
[21] Leslie Tung. A bi-domain model for describing ischemic myocardial dc potentials. PhD thesis, Massachusetts Institute of Technology, 1978.
[22] James P Keener and James Sneyd. Mathematical Physiology. Springer, 2009.
[23] Joakim Sundnes, Glenn Terje Lines, Xing Caᵢ, Bjorn Frederik Nielsen, Kent-Andre Mardal, and Aslak Tveito. Computing the electrical activity in the heart, volume 1. Springer Science & Business Media, 2007.
[24] Divya C Kernik, Stefano Morotti, HaoDi Wu, Priyanka Garg, Henry J Duff, Junko Kurokawa, José Jalife, Joseph C Wu, Eleonora Grandi, and Colleen E Clancy. A computational model of induced pluripotent stem-cell derived cardiomyocytes incorporating experimental variability from multiple data sources. The Journal ofphysiology, 2019.
[25] Joakim Sundnes, Glenn Terje Lines, and Aslak Tveito. An operator splitting method for solving the bidomain equations coupled to a volume conductor model for the torso. Mathematical biosciences, 194(2):233-248, 2005.
[26] H Joachim Schroll, Glenn Terje Lines, and Aslak Tveito. On the accuracy of operator splitting for the monodomain model of electrophysiology. International Journal of Computer Mathematics, 84(6):871-885, 2007.
[27] Joakim Sundnes, Robert Artebrant, Ola Skavhaug, and Aslak Tveito. A second-order algorithm for solving dynamic cell membrane equations. IEEE Transactions on Biomedical Engineering, 56(10):2546-2548, 2009.
[28] John A Nelder and Roger Mead. A simplex method for function minimization. The Computer Journal, 7(4):308-313, 1965.
[29] Stefan Otte, Sebastian Berg, Stefan Luther, and Ulrich Parlitz. Bifurcations, chaos, and sensitivity to parameter variations in the sato cardiac cell model. Communications in Nonlinear Science and Numerical Simulation, 37:265-281, 2016.
[30] Eric A Sobie. Parameter sensitivity analysis in electrophysiological models using multivariable regression. Biophysical journal, 96(4):1264-1274, 2009.
[31] Maria T Mora, Jose M Ferrero, Lucia Romero, and Beatriz Trenor. Sensitivity analysis revealing the effect of modulating ionic mechanisms on calcium dynamics in simulated human heart failure. PloS One, 12(11):e0187739, 2017.
[32] Karoline Horgmo Jaeger, Samuel Wall, and Aslak Tveito. Detecting undetectables: Can conductances of action potential models be changed without appreciable change in the transmembrane potential? Chaos: An Interdisciplinary Journal of Nonlinear Science, 29(7):073102, 2019.
[33] Rengasayee Veeraraghavan, Robert G Gourdie, and Steven Poelzing. Mechanisms of cardiac conduction: a history of revisions. American Journal of Physiology-Heart and Circulatory Physiology, 306(5):H619-H627, 2014.
[34] Jan P Kucera, Stephan Rohr, and Yoram Rudy. Localization of sodium channels in intercalated disks modulates cardiac conduction. Circulation research, 91(12):1176-1182, 2002.
[35] Jan P Kucera, Stephan Rohr, and Andre G Kleber. Microstructure, cell-to-cell coupling, and ion currents as determinants of electrical propagation and arrhythmogenesis. Circulation: Arrhythmia and Electrophysiology, 10(9):e004665, 2017.
[36] Karoline Horgmo Jaeger, Andrew G Edwards, Andrew McCulloch, and Aslak Tveito. Properties of cardiac conduction in a cell-based computational model. PLoS computational biology, 15(5):e1007042, 2019.
[37] Aslak Tveito, Karoline H Jaeger, Miroslav Kuchta, Kent-Andre Mardal, and Marie E Rognes. A cell-based framework for numerical modeling of electrical conduction in cardiac tissue. Frontiers in Physics, 5:48, 2017.
[38] Willemijn Groenendaal, Francis A Ortega, Armen R Kherlopian, Andrew C Zygmunt, Trine Krogh-Madsen, and David J Christini. Cell-specific cardiac electrophysiology models. PLoS computational biology, 11(4):e1004242, 2015.

1. A method for determining an effect of a drug on a cardiomyocyte (CM), the method comprising:
(a) obtaining, over time in control test tissue including cardiomyocytes (CMs), first measurements of transmembrane voltage (Vₘ), first measurements of intracellular calcium concentration (Caᵢ), and first measurements of extracellular potential (U);
(b) storing data representing said first measurements of Vₘ in a vector v^{C}, data representing said first measurements of Caᵢ in a vector c^{C}, and data representing said first measurements of U in a vector u^{C};
(c) contacting the control test tissue with a composition comprising a drug to produce a drug-treated test tissue including CMs;
(d) obtaining, over time in the drug-treated test tissue, second measurements of Vₘ, second measurements of Caᵢ, and second measurements of U;
(e) storing data representing said second measurements of Vₘ in vector v^{D}, data representing said second measurements of Caᵢ in vector c^{D}, and data representing said second measurements of U in vector u^{D};
(f) inverting the data stored in v^{C}, c^{C}, and u^{C} to update a value of one or more parameters in a base tissue model, stored in a vector p^{T,B}, to yield a vector p^{T,C} that parameterizes a control tissue model;
(g) inverting the data stored in v^{D}, C^{D}, and u^{D} to update the value of one or more parameters stored in the vector p^{T,C} to yield a vector p^{T,D} that parameterizes a drug-treated tissue model; and
(h) determining a change corresponding to a first parameter in the vector p^{T,D} relative to the first parameter in the vector p^{T,C}, thereby determining the effect of the drug.

2. The method of claim 1, wherein inverting the data comprises minimizing a cost function, and wherein the cost function measures a difference in shapes over time of the measured U, V, and Ca values relative to predicted values using a respective tissue model.

3. The method of claim 2, wherein the predicted values are determined by solving a partial differential equation of the respective tissue model, the partial differential equation including gradients of Vₘ and U.

4. The method of claim 2, wherein the cost function includes a term for the extracellular potential (U) and a term for a conduction velocity.

5. The method of claim 1, further comprising:
(i) updating one or more values in a diagonal maturation matrix Q such that an equality Qp^{T,C}=p^{M} is satisfied, wherein p^{M} is a vector that parameterizes a mature base tissue model and wherein before Q is updated, Q satisfies an equality p^{M}=Qp^{T,B}; and
(j) multiplying p^{T,D} by Q to generate vector p^{M,D}, wherein p^{M,D} parameterizes a mature drug-treated tissue model.

6. The method of claim 5, further comprising:
determining a difference between a parameter stored in p^{M,D} and a corresponding parameter in p^{M}; and
comparing the difference to a threshold to determine whether the effect of the drug is unacceptable.

7. The method of claim 6, wherein the at least one of the parameters includes a sodium current.

8. The method of claim 5, wherein determining the change further comprises:
(k) using the base tissue model and the drug-treated tissue model to generate a simulated mature control action potential (AP) and a simulated mature drug-treated AP, respectively; and
(l) comparing one or more morphological properties of the simulated mature control AP and the simulated mature drug-treated AP in order to calculate a difference, thereby determining the effect of the drug.

9. The method of claim 8, further comprising:
determining the drug does not have a proarrhythmic effect based on the difference; and
selecting the drug when it does not have a proarrhythmic effect.

10. The method of claim 1, wherein each of the control tissue model and the base tissue model is a bidomain model.

11. The method of claim 1, wherein the control test tissue includes stem cells.

12. The method of claim 11, wherein the stem cells are induced pluripotent stem cell-derived cardiomyocytes (iPSC-CM).

13. The method of claim 1, wherein the control test tissue includes animal cells of a first animal, wherein the base tissue model corresponds to a second animal, and wherein the first animal is of a different type than the second animal.

14. The method of claim 1, wherein the base tissue model corresponds to immature tissue including immature CMs, wherein the effect of the drug is determined for mature CMs by scaling the change.

15. The method of claim 1, further comprising:
comparing at least one of the parameters stored in p^{T,D} to a threshold to determine whether the effect of the drug is unacceptable.

16. The method of claim 5, further comprising:
determining a difference between a parameter stored in p^{T,D} and a corresponding parameter in p^{T,C}; and
comparing the difference to a threshold to determine whether the effect of the drug is unacceptable.

17. The method of claim 1, wherein steps (f) and (g) are performed simultaneously.

18. A method for determining an effect of a drug on a mature cardiomyocyte (CM) of a human, the method comprising:
(a) obtaining one or more first measurements of transmembrane voltage (Vₘ) and/or one or more first measurements of intracellular calcium concentration (Caᵢ) in an animal CM that is from an animal that is not human;
(b) storing data representing said one or more first measurements of Vₘ in vector v^{C} and/or storing data representing said one or more first measurements of Caᵢ in vector c^{C};
(c) contacting the animal CM with a composition comprising a sufficient amount of the drug to produce a drug-treated animal CM;
(d) obtaining one or more second measurements of Vₘ and/or one or more second measurements of Caᵢ in the drug-treated animal CM;
(e) storing data representing said one or more second measurements of Vₘ in vector v^{D} and/or storing data representing said one or more second measurements of Caᵢ in vector C^{D};
(f) inverting the data stored in v^{C} and/or c^{C} to update a value of one or more parameters in an animal base CM model, stored in vector p^{IM,B}, to yield vector p^{IM,C} that parameterizes an animal control CM model;
(g) inverting the data stored in v^{D} and/or c^{D} to update a value of one or more parameters stored in vector p^{IM,C} to yield vector p^{IM,D} that parameterizes an animal drug-treated CM model;
(h) updating one or more values in a diagonal maturation matrix Q such that the equality Qp^{IM,C}=p^{M} is satisfied, wherein p^{M} is a vector that parameterizes a mature base human CM model and wherein before Q is updated, Q satisfies the equality p^{M}=Qp^{IM,B};
(i) multiplying p^{IM,D} by Q to generate vector p^{M,D}, wherein p^{M,D} parameterizes a mature drug-treated human CM model;
(j) determining a change corresponding to a first parameter in the vector p^{M,D} relative to the first parameter in the vector p^{M}, thereby determining the effect of the drug.

19. The method of claim 18, wherein determining the change comprises:
(k) using the mature base human CM model and the mature drug-treated human CM model to generate a simulated mature control action potential (AP) and a simulated mature drug-treated AP, respectively; and
(l) comparing one or more morphological properties of the simulated mature control AP and the simulated mature drug-treated AP in order to calculate a difference, thereby determining the effect of the drug.

20. The method of claim 1, wherein the one or more parameters represent the number of one or more proteins in the CM model, dynamics of one or more proteins in the CM model, cell volume in the CM model, cell surface area in the CM model, cell membrane capacitance in the CM model, or a combination thereof.

21-27. (canceled)
